(19) United States
(12) Patent Application Publication
(10) Pub. No.: US 2025/0327822 A1
(43) Pub. Date: Oct. 23, 2025

(54) COMPANION DIAGNOSTIC ASSAY FOR GLOBO-H RELATED CANCER THERAPY

(71) Applicants: Ming-Tain LAI, Taipei City (TW); OBI PHARMA, INC., Taipei City (TW)

(72) Inventors: Cheng-Der Tony YU, Taipei City (TW); Ming-Tain LAI, Taipei City (TW); I-Ju CHEN, Taipei City (TW); Yu-Jung CHEN, Taipei City (TW); Ming-Chen YANG, Taipei City (TW)

(21) Appl. No.: 17/595,830

(22) PCT Filed: Mar. 27, 2020
(86) PCT No.: PCT/US2020/025516

§ 371 Date: Nov. 24, 2021

Related U.S. Application Data
(60) Provisional application No. 62/825,625, filed on Mar. 28, 2019.

(51) Int. Cl. G01N 33/92

(52) U.S. Cl. CPC G01N 33/92; G01N 2405/10; G01N 2474/20; G01N 2800/52

(57) ABSTRACT

Methods and reagents suitable for in vitro diagnostic assay comprising a qualitative immunohistochemical assay using anti-Globo H antibodies and/or binding fragments thereof are provided. The method comprises the detection of Globo-H expression levels in formalin-fixed, paraffin-embedded (FFPE) cancer tissue using a visualization system. The Globo-H expression can be determined by using tumor scoring showing partial or complete staining at any intensity.

### RELATED APPLICATIONS

This application claims the benefit and priority to U.S. Provisional Application No. 62/825,625, filed on Mar. 28, 2019, entitled, “COMPANION DIAGNOSTIC ASSAY FOR GLOBO-H RELATED CANCER THERAPY”, the contents of which is incorporated by reference herewith in its entirety.

### SEQUENCE LISTING

The instant application contains a Sequence Listing which has been submitted electronically in ASCII cormat and is hereby incorporated by reference in its entirety. Said ASCII copy, created on Nov. 23, 2021, is named G3004-01501NP_SL.txt and is 20,893 bytes in size.

### FIELD OF THE INVENTION

The present disclosure relates to methods and reagents and kits for detecting Globo-H levels in cancer/patients/specimens for selecting patients to receive Globo-H related therapy and for monitoring patient response to Globo-H mediated therapy. Exemplary tissue samples include breast tissue specimen, pancreatic specimen, lung specimen, gastric specimen, liver specimen, colorectal specimen, and esophageal specimen. The methods of the invention allow more effective identification of patients to receive Globo-H mediated therapy and of determination of patient response to the therapy.

### BACKGROUND OF THE INVENTION

Numerous surface carbohydrates are expressed in malignant tumor cells. For example, the carbohydrate antigen Globo-H (Fucα1→2 Galβ1→3 GalNAcβ1→3 Galα1→4 Galβ1→4 Glc) was first isolated as a ceramide-linked Glycolipid and identified in 1984 from breast cancer MCF-7 cells. (Bremer E G, et al. (1984) J Biol Chem 259:14773-14777). Previous studies have also shown that Globo-H and stage-specific embryonic antigen 3 (Galβ1→3GalNAcβ1→3Gal α1→4Galβ1→4Glcβ1) (SSEA-3, also called Gb5) were observed on breast cancer cells and breast cancer stem cells (WW Chang et al. (2008) Proc Natl Acad Sci USA, 105 (33): 11667-11672). In addition, SSEA-4 (stage-specific embryonic antigen-4) (Neu5Acα2→3Galβ1→3GalNAcβ1→3Galα1→4Galβ1→4Glcβ1) has been commonly used as a cell surface marker for pluripotent human embryonic stem cells and has been used to isolate mesenchymal stem cells and enrich neural progenitor cells (Kannagi R et al. (1983) EMBO J, 2:2355-2361).

### SUMMARY OF THE INVENTION

Globo series antigens (Globo-H, SSEA-3 and SSEA-4) are uniquely expressed on cancer cells and can facilitate targeting of anti-cancer therapeutic agents to cancer cells with high specificity. Globo series antigens can serve as glycan markers associated with and/or predictive of cancers, and develop antibody and or binding fragments thereof against the markers for use in diagnosing and treating a broad spectrum of cancers.

Accordingly, due to the potential therapeutic use of Globo-H related treatment modalities, companion diagnostic assays that would identify patients eligible to receive Globo-H mediated therapy are needed. Additionally, there is a clear need to support this therapy with diagnostic assays using glycan markers that would facilitate monitoring the efficacy of Globo-H mediated treatment modalities.

Accordingly, the present disclosure is based on innovative methods for detecting Globo series antigens demonstrated to be aberrantly expressed in a broad spectrum of cancers, but not on normal cells. Cancers expressing Globo series antigens include, but are not limited to, sarcoma, skin cancer, leukemia, lymphoma, brain cancer, glioblastoma, lung cancer, breast cancer, gastric cancer, oral cancer, head-and-neck cancer, nasopharyngeal cancer, esophagus cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testical cancer, buccal cancer, oropharyngeal cancer, laryngeal cancer and prostate cancer.

The present invention relates to the detection, identification and/or use of Globo-H expression patterns (or profiles or signatures), which are clinically relevant to cancer therapy. In particular, disclosed herein are markers that can be used to identify, treat and monitor patients for cancer treatment, and particularly anti-Globo H therapy.

The present invention provides companion methods of detecting/diagnostic assays for classification of patients for cancer treatment, the methods comprising detecting, quantifying and/or assessing Globo-H carbohydrate antigen levels in a patient tissue sample using the innovative lab techniques of this disclosure. The inventive assays include assay methods for identifying patients eligible to receive anti-Globo H therapy and for monitoring patient response to such therapy. In some aspects, the inventive methods comprise detecting, quantifying and/or assessing carbohydrate antigens and/or carbohydrate modified proteins in samples by immunohistochemistry or in situ hybridization assays.

Exemplary anti-Globo H agents can include antibodies and/or fragments thereof. In certain embodiment, the anti-Globo H antibody is OBI-888 (Anti-Globo H monoclonal antibody) Exemplary OBI-888 is as described in PCT patent publications (WO2015157629A2 and WO2017062792A1), US patent and patent applications (U.S. Pat. No. 9,902,779B2, US2017101462A1 and US20180134799A1), the contents of which are incorporated by reference in its entirety.

The amino acid sequences of the variable heavy chains and the variable light chains from OBI-888 (Anti-Globo H monoclonal antibody) hybridoma clones were shown in the following Table A (TABLE-A):

| | | |
|---|---|---|
| | | SEQ |
| Clone name | Amino acid sequence | ID No. |

Heavy Chain Variable Region (VH)

| | | |
|---|---|---|
| 2C2-VH | SGPGILQPSQTLSLTCSFSGFSLYTFDMGVGWIRQPSGKGLEWLA | 1 |
| | HIWWDDDKYYNPALKSRLTVSKDTSKNQVFLKIPNVDTADSATYY | |
| | CARVRGLHDYYYWFAYWGQGTLVTVS | |
| 3D7-VH | SGPGILQPSQTLSLTCSFSGFSLYTFDMGVGWIRQPSGKGLEWLA | 2 |
| | HIWWDDDKYYNPALKSRLTVSKDTSKNQVFLKIPNVDTADSATYY | |
| | CARVRGLHDYYYWFAYWGQGTLVTVS | |
| 7A11-VH | SGPGILQPSQTLSLTCSFSGFSLYTFDMGVGWIRQPSGKGLEWLA | 3 |
| | QIWWDDDKYYNPGLKSRLTISKDTSKNQVFLKIPNVDTADSATYYC | |
| | ARIRGLRDYYYWFAYWGQGTLVTVS | |
| 2F8-VH | SGPGILQPSQTLSLTCSFSGFSLSTFGLGVGWIRQPSGKGLEWLAHI | 4 |
| | WWDDDKSYNPALKSRLTISKDTSKNQVFLMIANVDTADTATYYCA | |
| | RIGPKWSNYYYYCDYWGQGTTLTVS | |
| 1E1-VH | SGPGILQPSQTLSLTCSFSGFSLSTFGLGVGWIRQPSGKGLEWLAHI | 5 |
| | WWDDDKSYNPALKSQLTISKDTSKNQVLLKIANVDTADTATYYCA | |
| | RIGPKWSNYYYYCDYWGQGTTLTVS | |
| Humanized | QITLKESGPTLVKPTQTLTLTCTFSGFSLYTFDMGVGWIRQPPGKG | 6 |
| Antibody- | LEWLAHIWWDDDKYYNPALKSRLTISKDTSKNQVVLTMTNMDPV | |
| VH | DTATYYCARVRGLHDYYYWFAY | |
| Chimeric | QVTLKESGPGILQPSQTLSLTCSFSGFSLYTFDMGVGWIRQPSGKG | 7 |
| Antibody- | LEWLAHIWWDDDKYYNPALKSRLTVSKDTSKNQVFLKIPNVDTAD | |
| VH | SATYYCARVRGLHDYYYWFAY | |
| R28-VH | QITLKESGPTLVKPTQTLTLTCTFSGFSLYTFDMGVGWIRQPPGKG | 8 |
| | LEWLAHIWWDGDKYYNPALKSRLTISKDTSKNQVVLTMTNMDP | |
| | VDTATYYCARVRGLHRYYYWFAYWGQGTLVTVSSASTKGPSVFPL | |
| | APSSKSTSGGTAALGCLVKDYFPEPVTVSWNSGALTSGVHTFPAVL | |
| | QSSGLYSLSSVVTVPSSSLGTQTYICNVNHKPSNTKVDKKVEPKSCD | |
| | KTHTCPPCPAPELLGGPSVFLFPPKPKDTLMISRTPEVTCVVVDVSH | |
| | EDPEVKFNWYVDGVEVHNAKTKPREEQYNSTYRVVSVLTVLHQD | |
| | WLNGKEYKCKVSNKALPAPIEKTISKAKGQPREPQVYTLPPSRDELT | |
| | KNQVSLTCLVKGFYPSDIAVEWESNGQPENNYKTTPPVLDSDGSF | |
| | FLYSKLTVDKSRWQQGNVFSCSVMHEALHNHYTQKSLSLSPGK | |

Light Chain Variable Region (VL)

| | | |
|---|---|---|
| 2C2-VL | ASPGEKVTMTCRASSSVSYMHWYQQKPGSSPKPWIYATSNLASG | 9 |
| | VPARFSGSGSGTSYSLTISRVEAEDAATYFCQQWSRNPFTFGSGTK | |
| | LEIR | |
| 3D7-VL | ASPGEKVTMTCRASSSVSYMHWYQQKPGSSPKPWIYATSNLASG | 10 |
| | VPARFSGSGSGTSYSLTISRVEAEDAATYFCQQWSRNPFTFGSGTK | |
| | LEIR | |
| 7A11-VL | ASPGEKVTMTCRASSSVSYMHWYQQKPGSSPKPWIYATSNLASG | 11 |
| | VPARFSGSGSGTSYSLTISRVEAEDAATYFCQQWSRNPFTFGSGTK | |
| | LEIR | |
| 2F8-VL | ASPGEKVTMTCRASSSVSYMHWYQQKPGSSPKPYIYATSNLSSGV | 12 |
| | PARFSGSGSGTSYSLTISRVEAEDAATYYCQQWSSNPFTFGSGTKL | |
| | EIK | |
| 1E1-VL | ASPGEKVTMTCRASSSVSYMHWYQQKPGSSPKPYIYATSNLSSGV | 13 |
| | PARFSGSGSGTSYSLTISRVEAEDAATYYCQQWSSNPFTFGSGTKL | |
| | EIK | |
| Humanized | EIVLTQSPATLSLSPGERATLSCRASSSVSYMHWYQQKPGKSPKP | 14 |
| Antibody-VL | WIYATSNLASGVPSRFSGSGSGTDFTFTISSLQPEDIATYYCQQWS | |
| | RNPFT | |
| Chimeric | QIVLSQSPTILSASPGEKVTMTCRASSSVSYMHWYQQKPGSSPKP | 15 |
| Antibody-VL | WIYATSNLASGVPARFSGSGSGTSYSLTISRVEAEDAATYFCQQWS | |
| | RNPFT | |
| R28-VL | EIVLTQSPATLSLSPGERATLSCRASSSVSYMHWYQQKPGKSPKP | 16 |
| | WIYATSNKASGVPSRFSGSGSGTDFTFTISSLQPEDIATYYCQQWS | |
| | RRPFTFGQGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGEC | |

In one aspect, the present disclosure provides a method of classifying a patient for eligibility for cancer therapy with an anti-Globo H antibody or a binding fragment thereof comprising: (a) providing a tissue sample from a patient; (b) detecting Globo-H expression level in said sample; and (c) classifying the patient as eligible to receive a cancer therapy with anti-Globo H therapy based on Globo-H expression level in said sample.

In one embodiment, the tissue sample comprises a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample.

In one embodiment, the determining step (b) is performed by IHC, in situ hybridization, by polymerase chain reaction or by a microarray assay.

In one embodiment, the cancer therapy comprises treatment with anti-Globo H monoclonal antibody or binding fragment thereof.

In one embodiment, the method further comprising detecting Globo-H expression level in the sample by IHC.

In one embodiment, the cancer is selected from the group consisting of breast cancer, lung cancer, gastric cancer, colorectal cancer, liver cancer, and esophageal cancer.

In one aspect, the present disclosure provides a method for identifying a patient with cancer as eligible to receive anti-Globo H therapy comprising: (a) providing a tissue sample from a patient; (b) detecting levels in the tissue sample of Globo-H; and (c) classifying the patient as eligible to receive anti-Globo H therapy where the tissue sample is classified as having increased and/or decreased levels of Globo-H compared to levels in a normal control sample.

In one embodiment, the tissue sample comprises a peripheral blood sample, a tumor or suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor or suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample a fresh frozen tissue sample or a paraffin embedded tissue sample.

In one embodiment, the patient is classified as eligible to receive anti-Globo H antibody or a binding fragment thereof.

In one embodiment, the patient is classified as eligible to receive anti-Globo H combination therapy.

In one embodiment, the determining step (b) is performed by IHC.

In one embodiment, the cancer is selected from the group consisting of breast cancer, lung cancer, gastric cancer, colorectal cancer, liver cancer, and esophageal cancer.

In one aspect, the present disclosure provides a method for monitoring a patient being treated with anti-Globo H therapy comprising: (a) providing a peripheral blood sample from a cancer patient; (b) identifying in or extracting from the peripheral blood sample circulating tumor cells; (c) determining in the circulating tumor cells Globo-H levels; and (d) comparing the Globo-H status in circulating tumor cells determined before or at onset of therapy.

In one embodiment, the cancer is selected from the group consisting of breast cancer, lung cancer (e.g. NSCLC), gastric cancer, colorectal cancer, liver cancer, and esophageal cancer.

In one embodiment, the patient is being treated with anti-Globo H agents.

In one embodiment, the patient is being treated with an anti-Globo H antibody or a binding fragment thereof.

In one embodiment, the determining step (c) is performed by IHC and/or in situ hybridization.

In one aspect, the present disclosure provides a kit comprising an anti-Globo H antibody and/or binding fragment composition for IHC comprising primary antibody and labeled secondary antibody wherein the primary antibody is designed to hybridize specifically under selected high stringency conditions to Globo-H target by IHC, wherein the secondary antibody is designed to bind to the primary antibody wherein the secondary antibody is linked to dextran polymer with HRP molecules wherein when applied the detection level of Globo-H in the sample is greater than 80%, 81%, 82%, 83%, 84%, 85%, 86%, 87%, 88%, 89%, or 90% or more.

In one embodiment, the primary and secondary antibodies in the kit can be in separate container.

In one embodiment, the kit further comprises an instruction for use further comprising one or more of a buffering solution, a blocking reagent, a negative control reagent, a linker, a visualization reagent (e.g., HRP).

The details of one or more embodiments of the invention are set forth in the description below. Other features or advantages of the present invention will be apparent from the following drawings and detailed description of several embodiments, and also from the appending claims.

### BRIEF DESCRIPTION OF THE FIGURES

A more complete understanding of the invention may be obtained by reference to the accompanying drawings, when considered in conjunction with the subsequent detailed description. The embodiments illustrated in the drawings are intended only to exemplify the invention and should not be construed as limiting the invention to the illustrated embodiments.

### Figures in Example 1

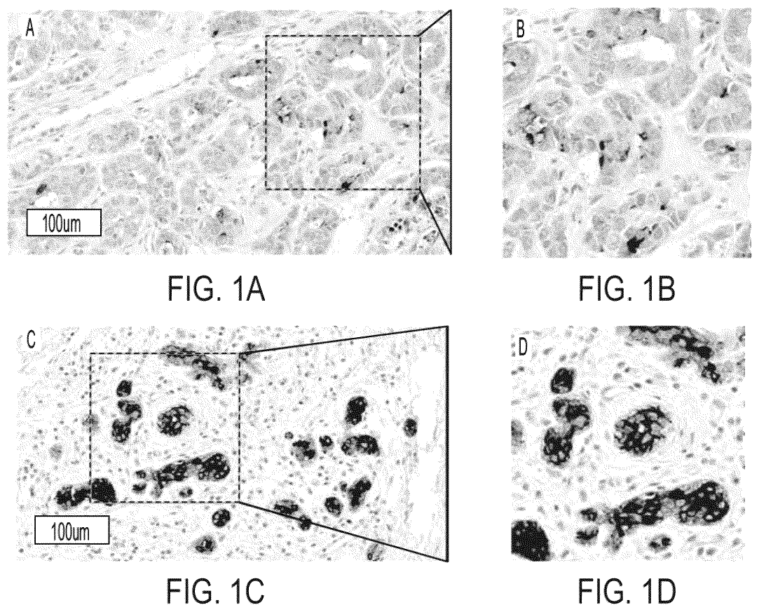

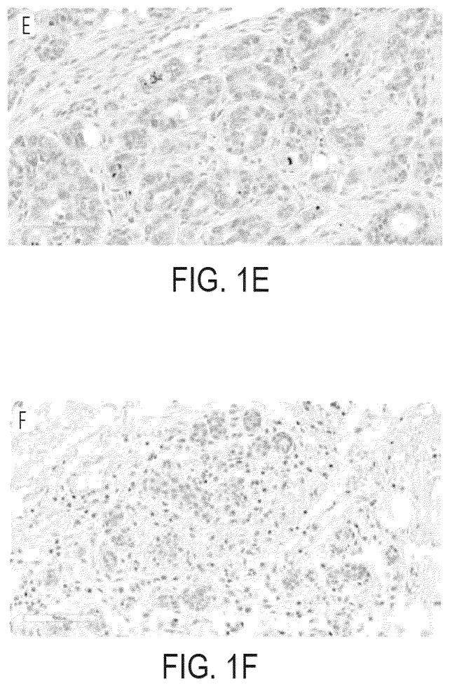

FIGS. 1A to 1F show the Globo-H IHC staining of the breast cancer control tissue.

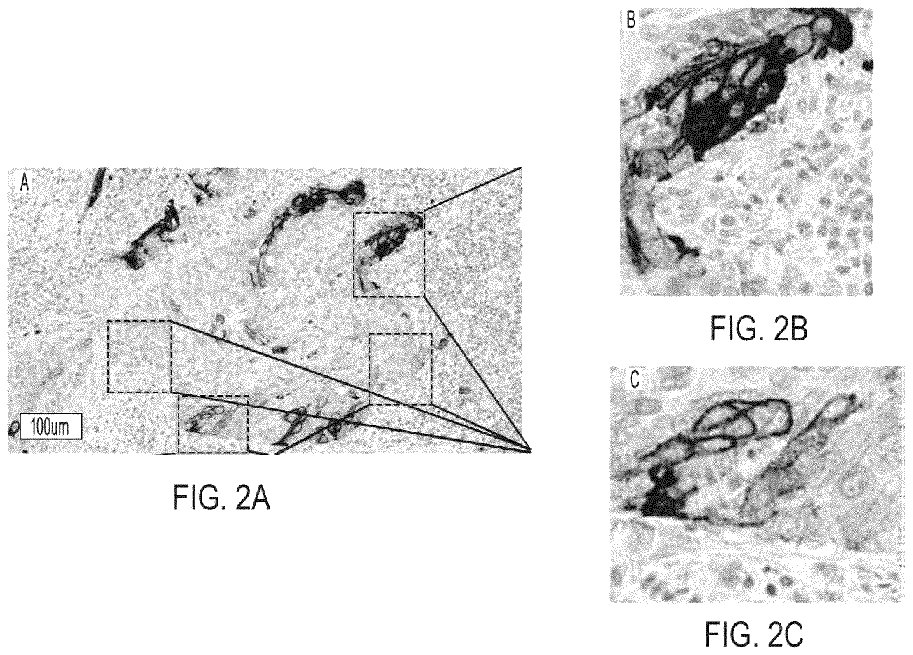

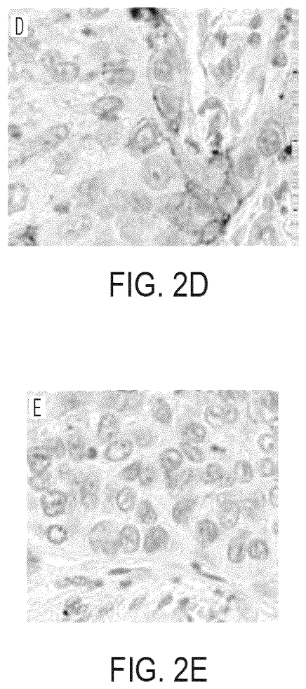

FIGS. 2A to 2E show the Globo-H IHC staining intensities in a NSCLC specimen.

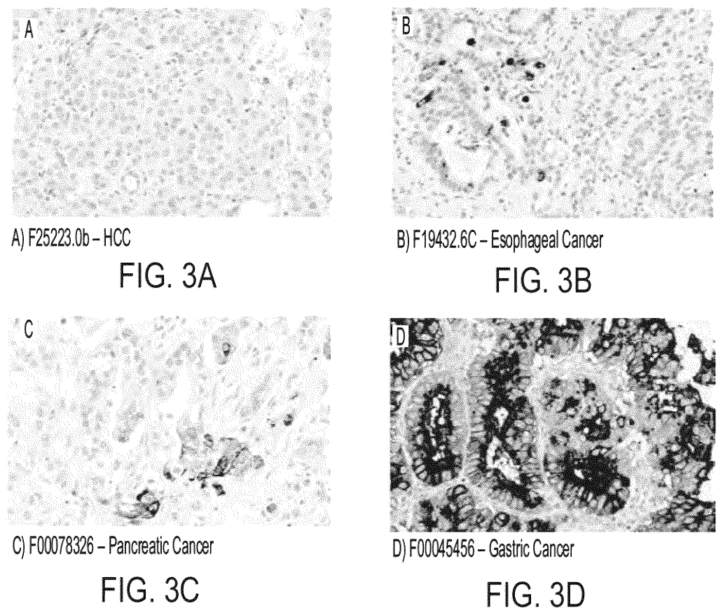

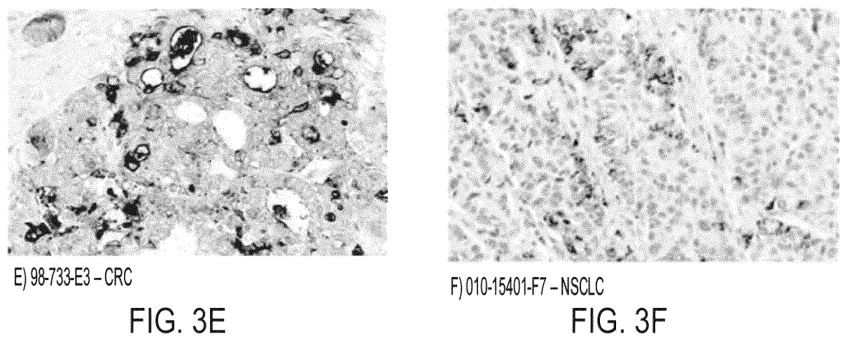

FIGS. 3A to 3F show images of the Confirmed Globo-H IHC Assay.

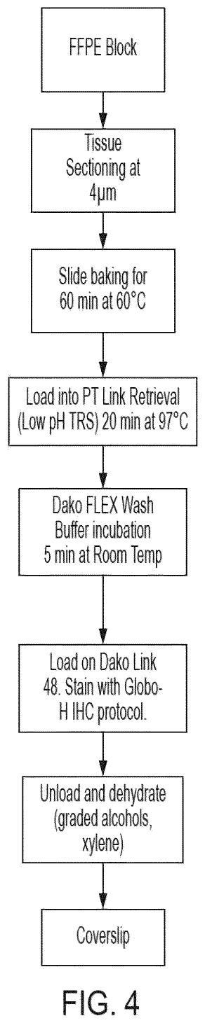

FIG. 4 shows the Globo-H IHC Specimen Work Flow.

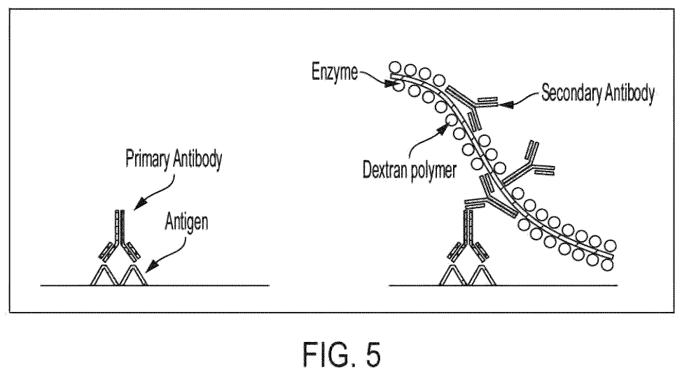

FIG. 5 shows the EnVision FLEX Detection System Binding Schematic.

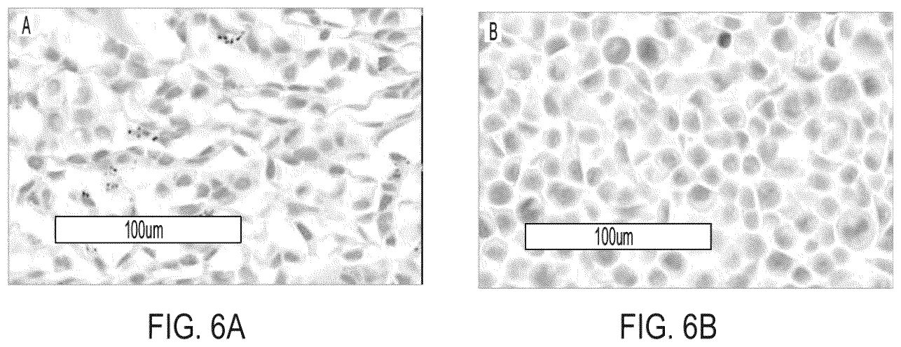

FIGS. 6A and 6B show the Globo-H IHC in HPAC and SK-BR3 Cell Lines.

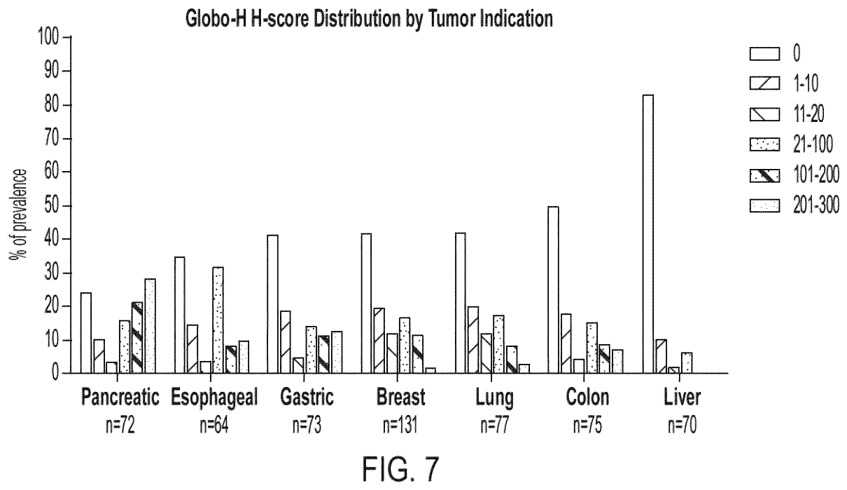

FIG. 7 shows the Globo-H IHC H-score Distribution of Validation Tumor Specimens Per Indication.

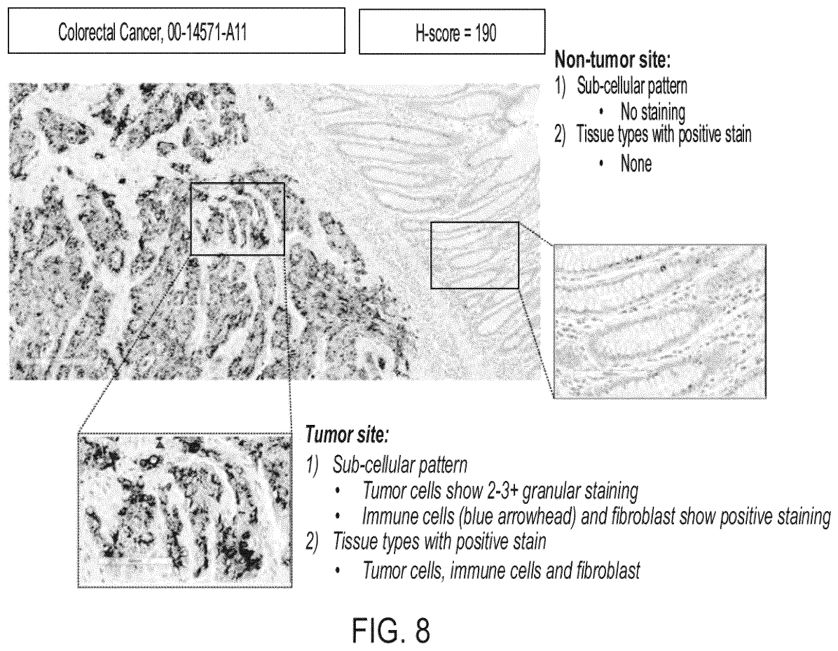

FIG. 8 shows the Schematic Representative Image of Globo-H IHC in a CRC Resection Specimen.

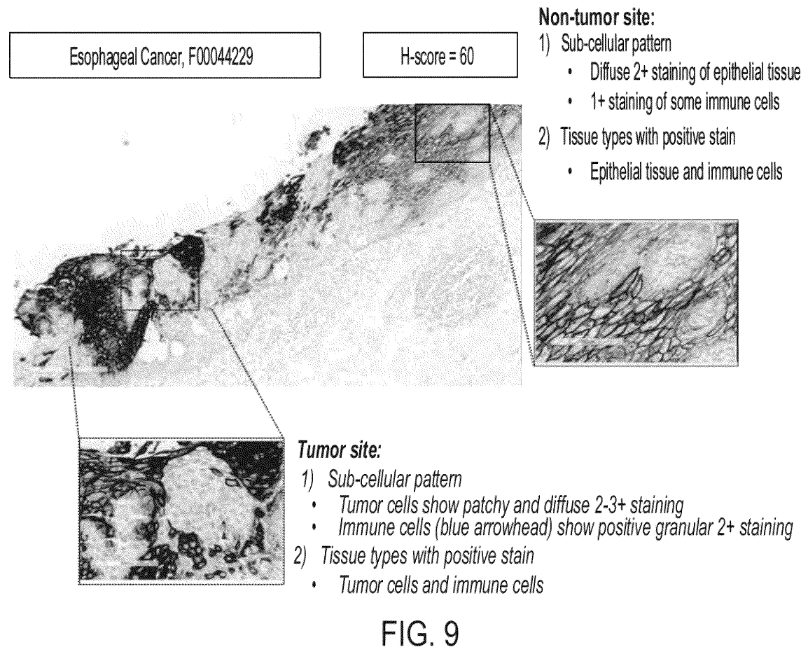

FIG. 9 shows the Schematic Representative Image of Globo-H IHC in an Esophageal Cancer Resection Specimen.

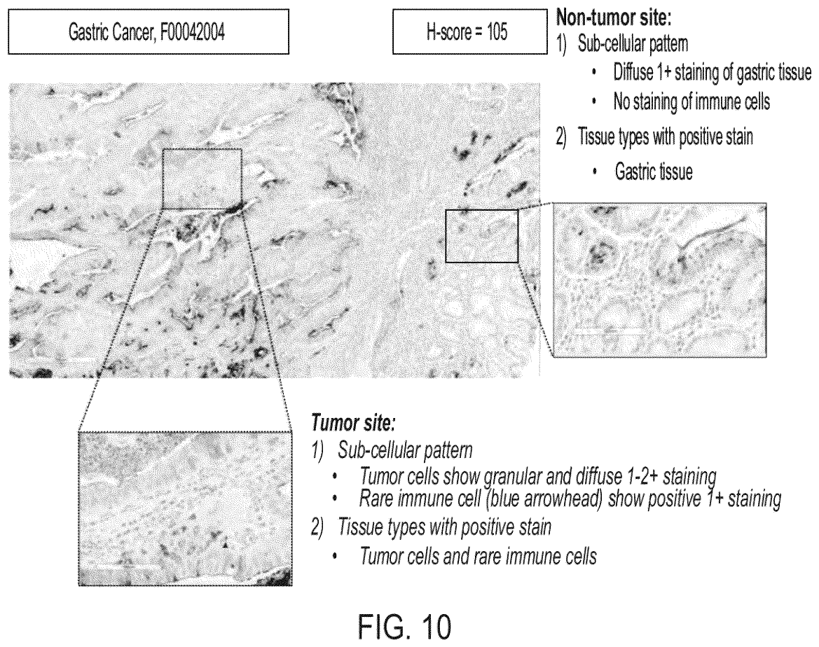

FIG. 10 shows the Schematic Representative Image of Globo-H IHC in a Gastric Cancer Resection Specimen.

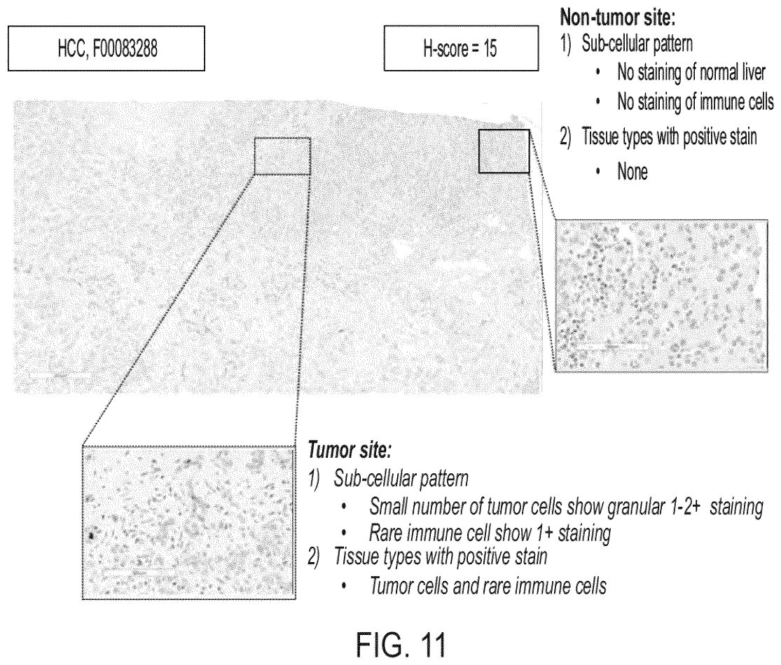

FIG. 11 shows the Schematic Representative Image of Globo-H IHC in a HCC Resection Specimen.

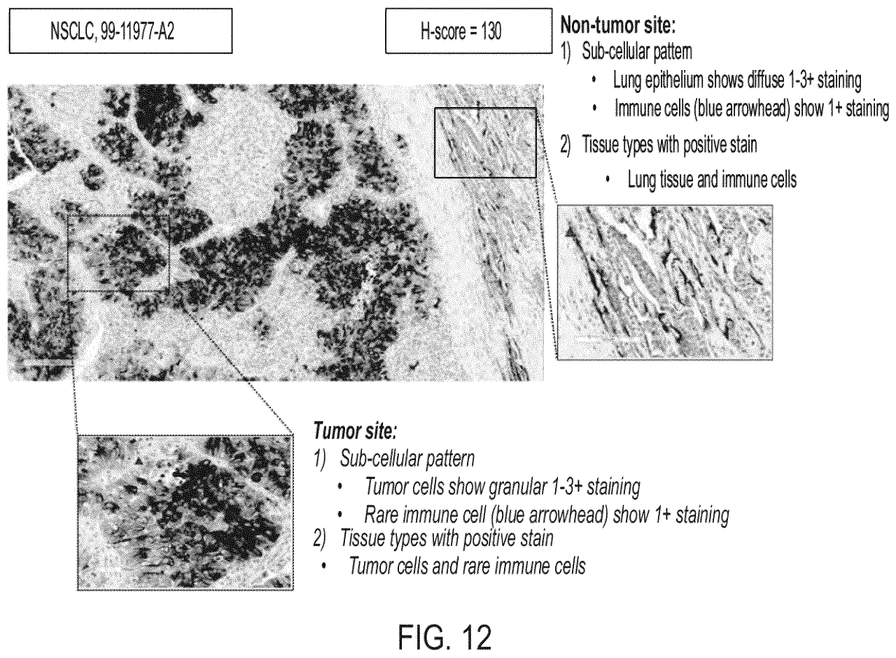

FIG. 12 shows the Schematic Representative Image of Globo-H IHC in a NSCLC Resection Specimen.

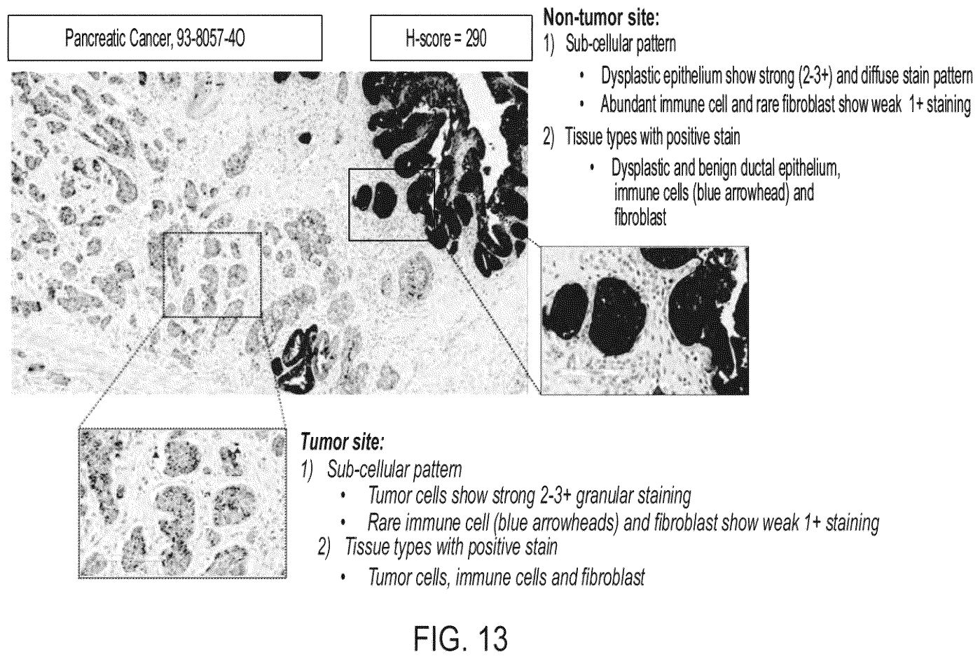

FIG. 13 shows the Schematic Representative Image of Globo-H IHC in a Pancreatic Cancer Resection Specimen.

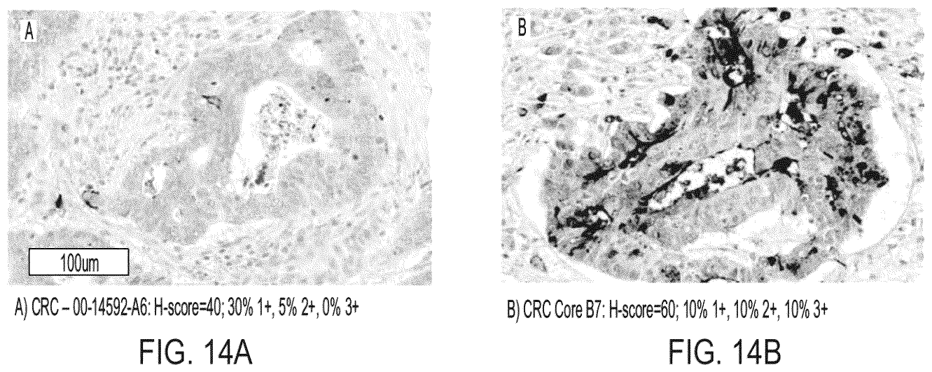

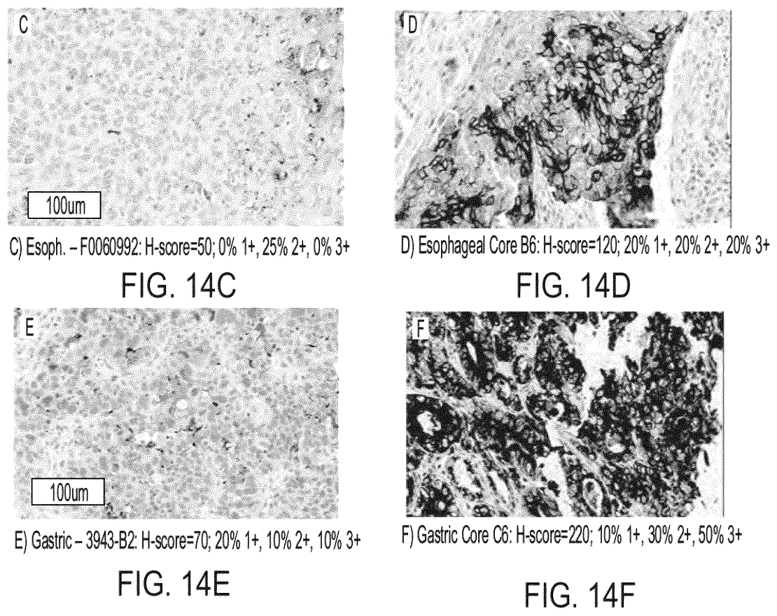

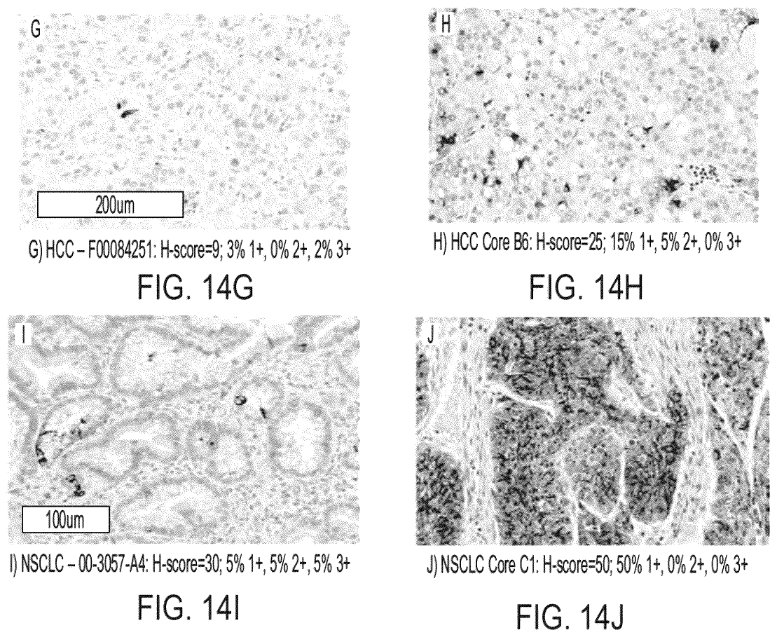

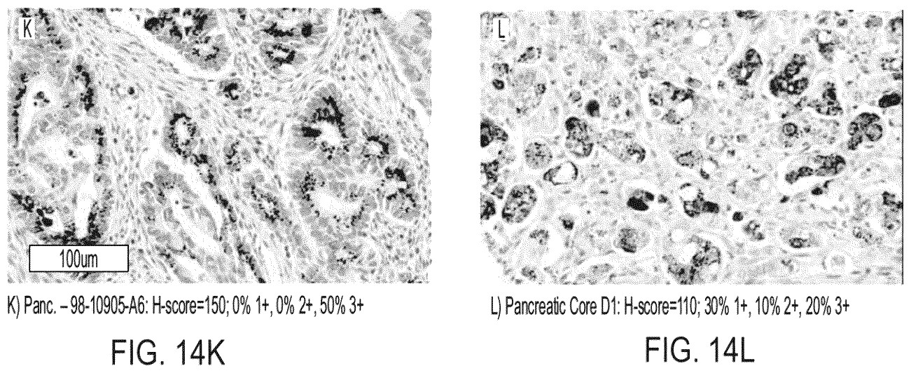

FIGS. 14A to 14L show the Globo-H IHC in Whole Specimens and TMA cores from 6 Tumor Indications.

### Figures in Example 2

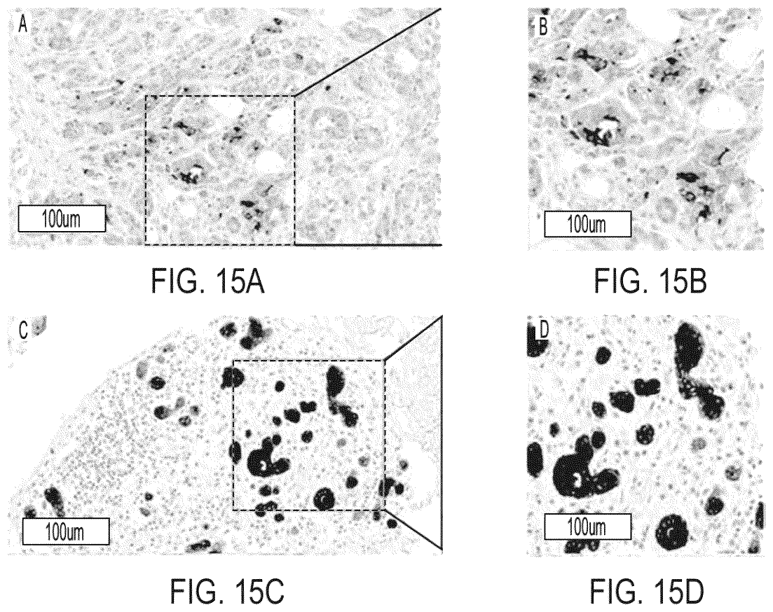

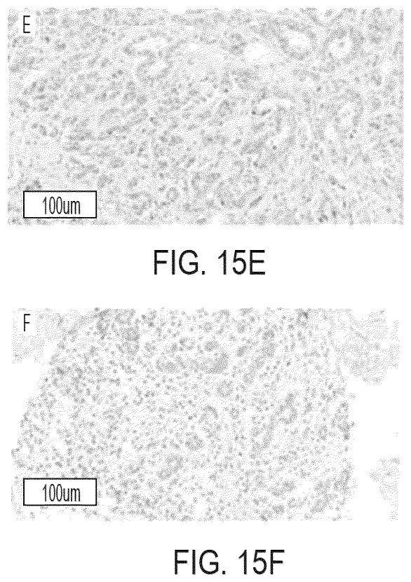

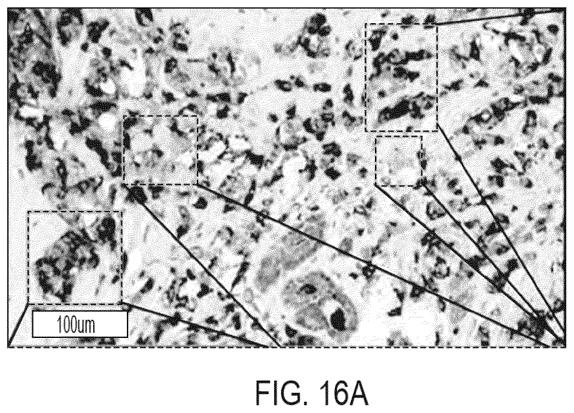

FIGS. 15 to 15F show the Globo-H IHC staining of the breast cancer control tissue.

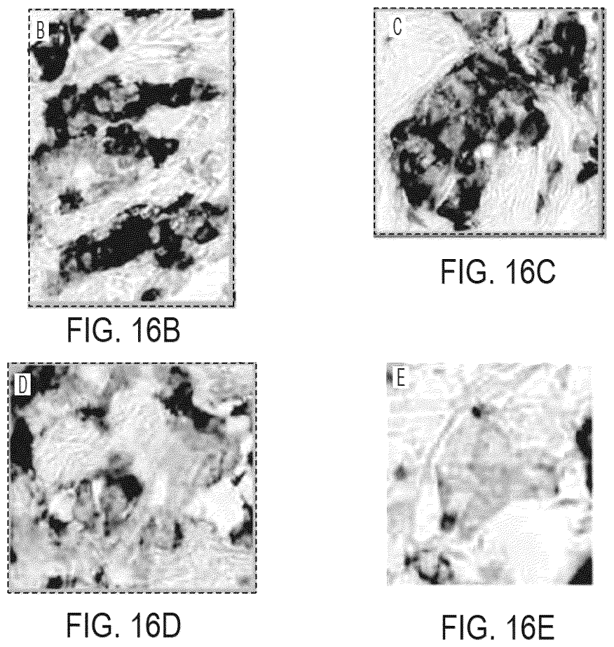

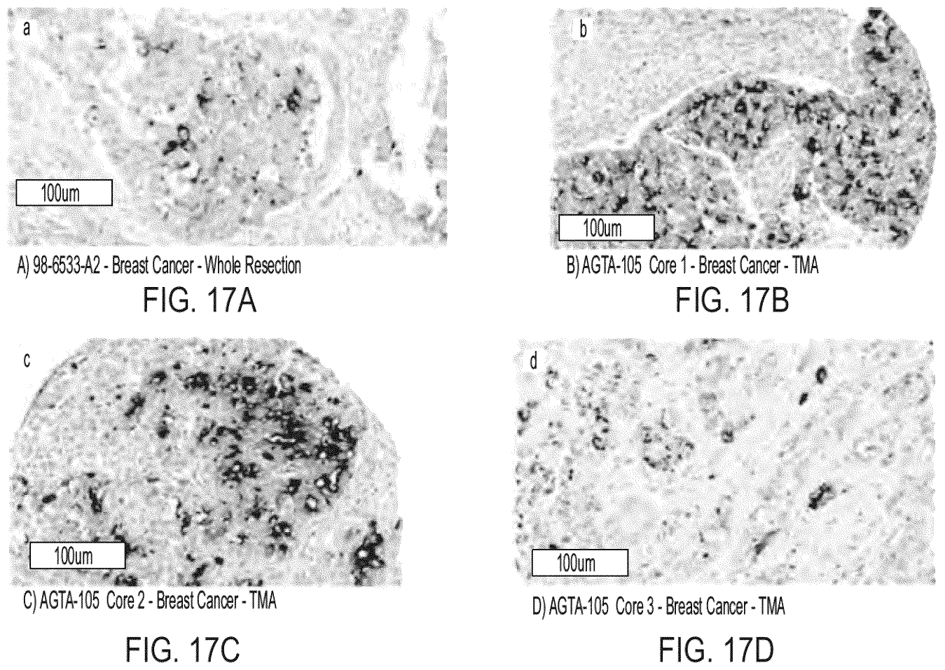

FIGS. 16A to 16E show the Globo-H IHC staining intensities in a breast cancer specimen.

FIGS. 17A to 17D show Images of the Optimized Globo-H IHC Assay.

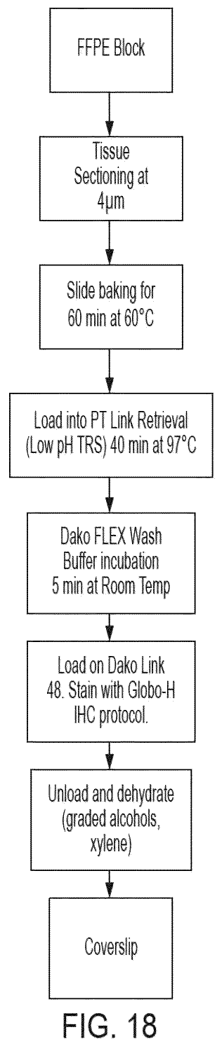

FIG. 18 shows the Globo-H IHC Specimen Work Flow.

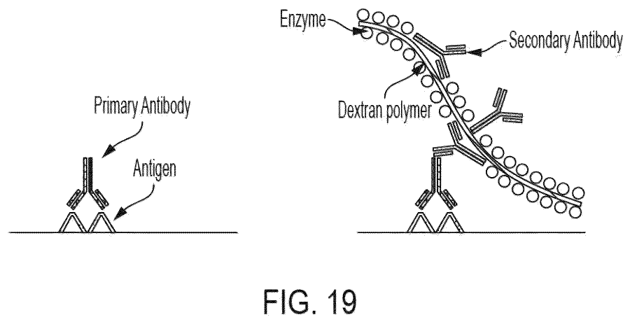

FIG. 19 show the En Vision FLEX Detection System Binding Schematic.

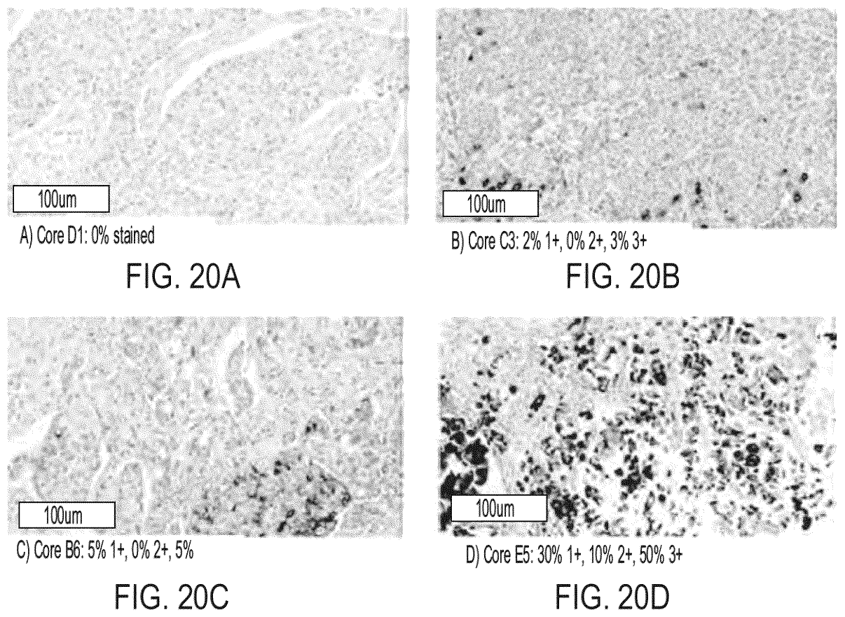

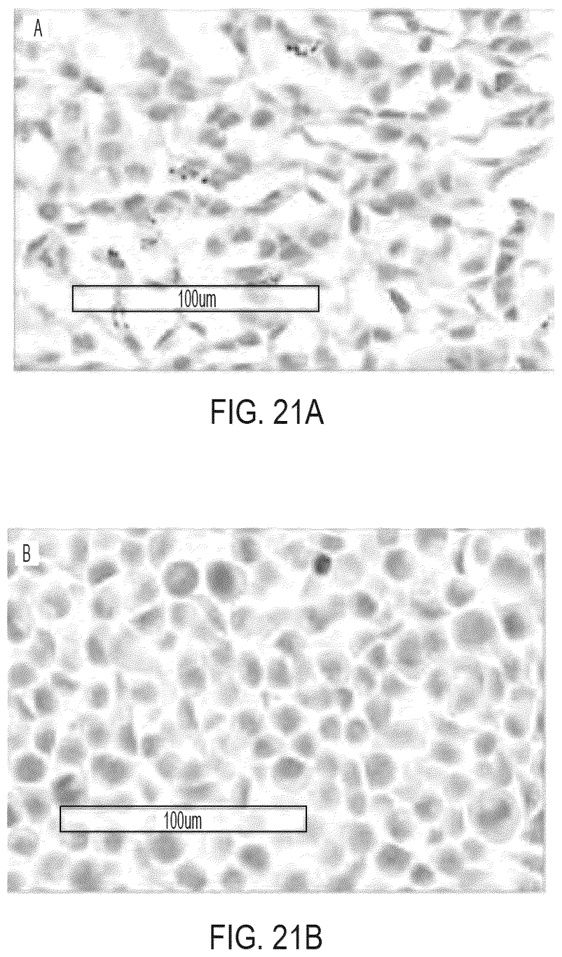

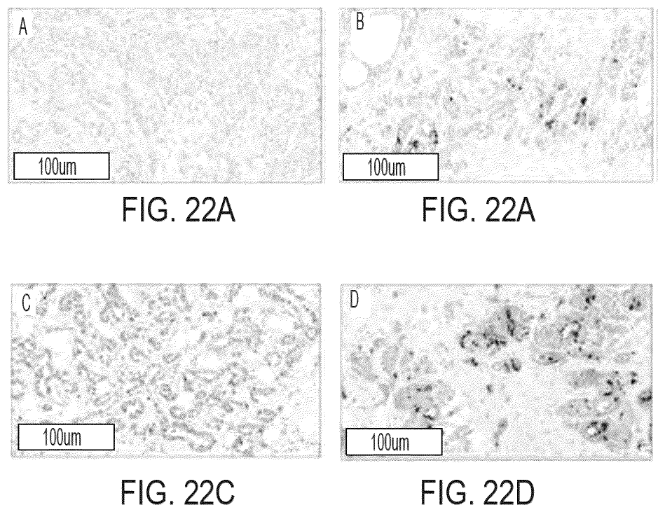

FIGS. 20A to 20D show the Globo-H IHC in Breast Cancer TMA Cores.

FIGS. 21A to 21B show the Globo-H IHC in HPAC and SK-BR3 Cell Lines.

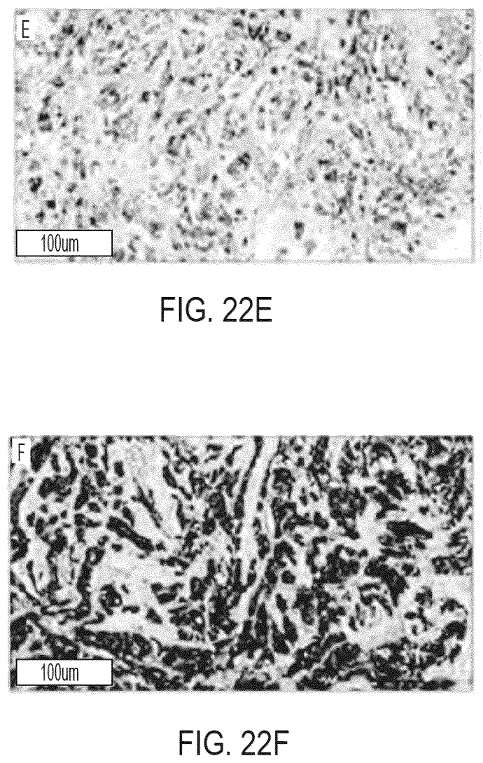

FIGS. 22A to 22F show the Globo-H IHC in Breast Cancer Specimens.

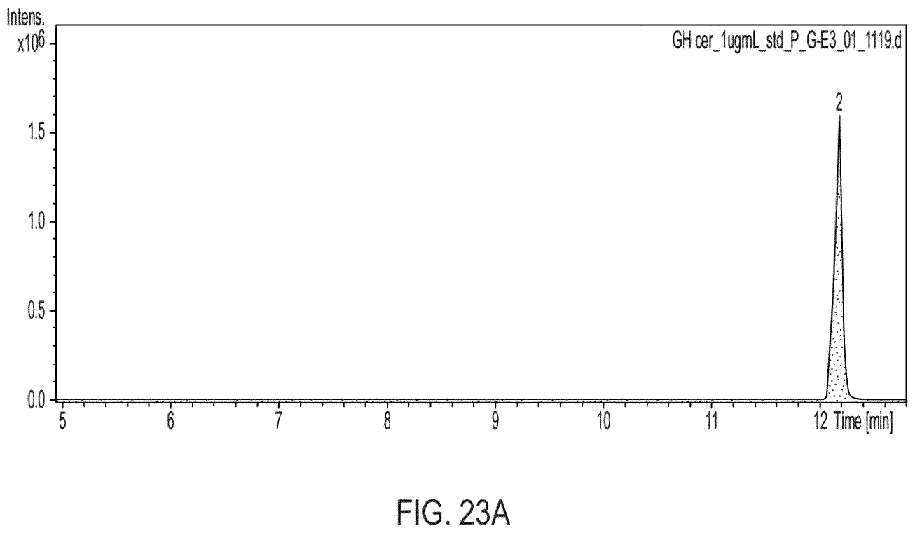

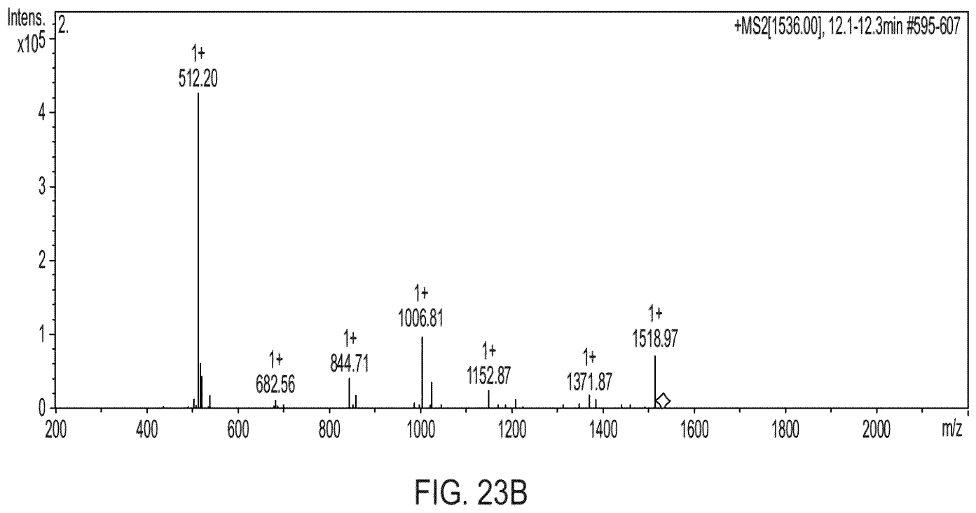

FIG. 23 shows The Extract ion chromatogram of 1536→512 and MS/MS spectrum of Globo-H ceramide.

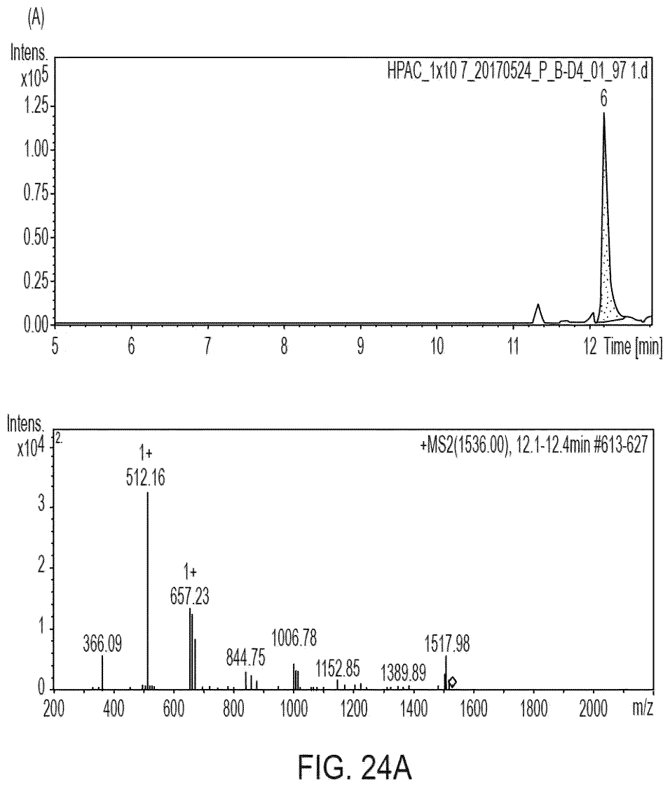

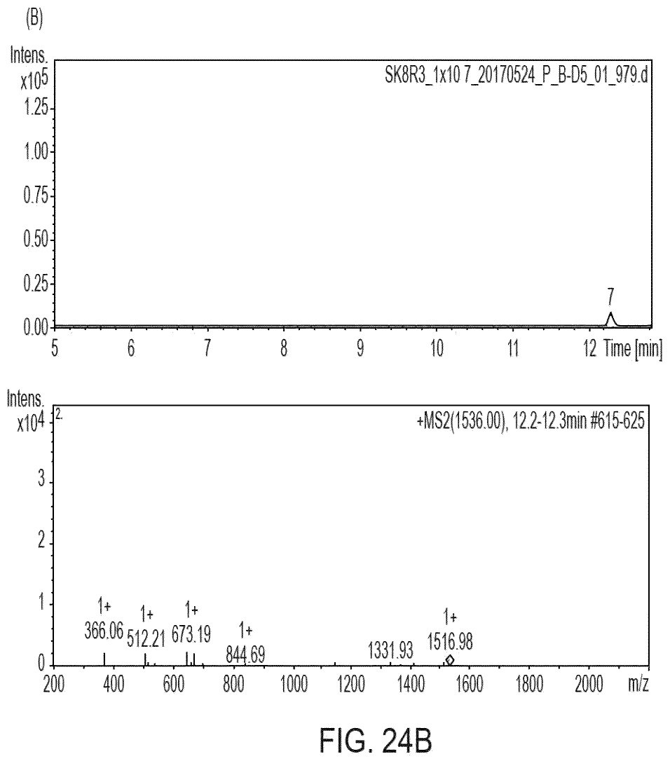

FIGS. 24A to 24B show the results of LC-MS/MS HPAC and SKBR3.

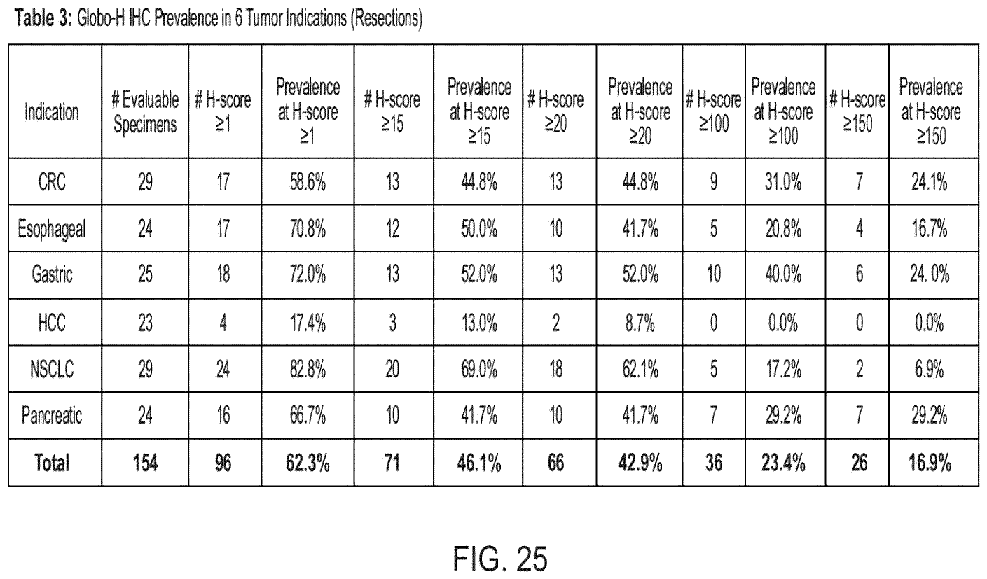

FIG. 25 shows Table 3

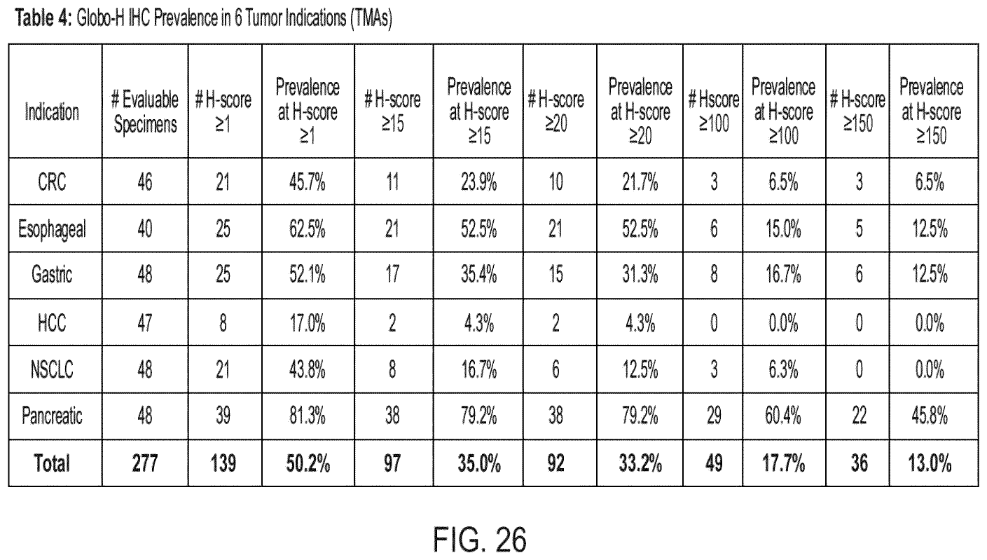

FIG. 26 shows Table 4.

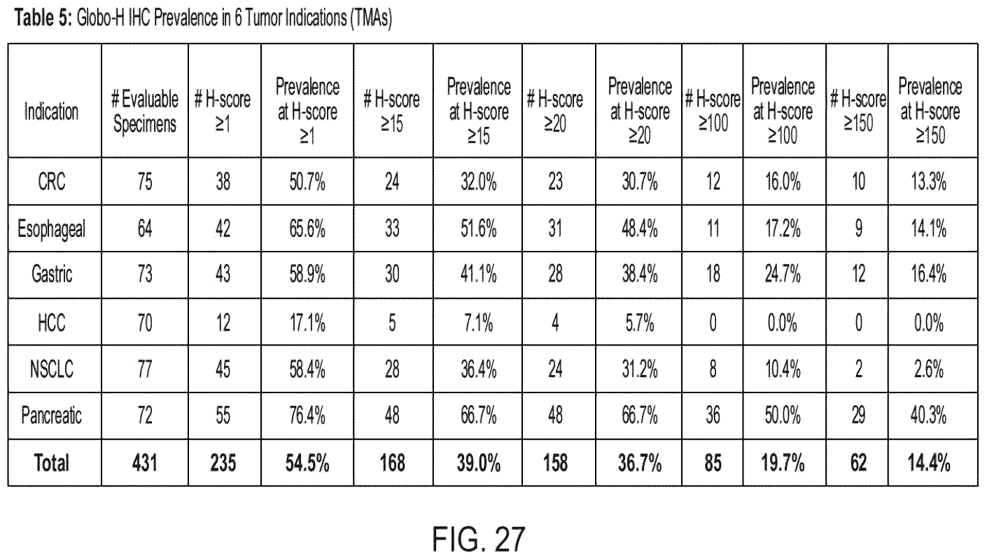

FIG. 27 shows Table 5.

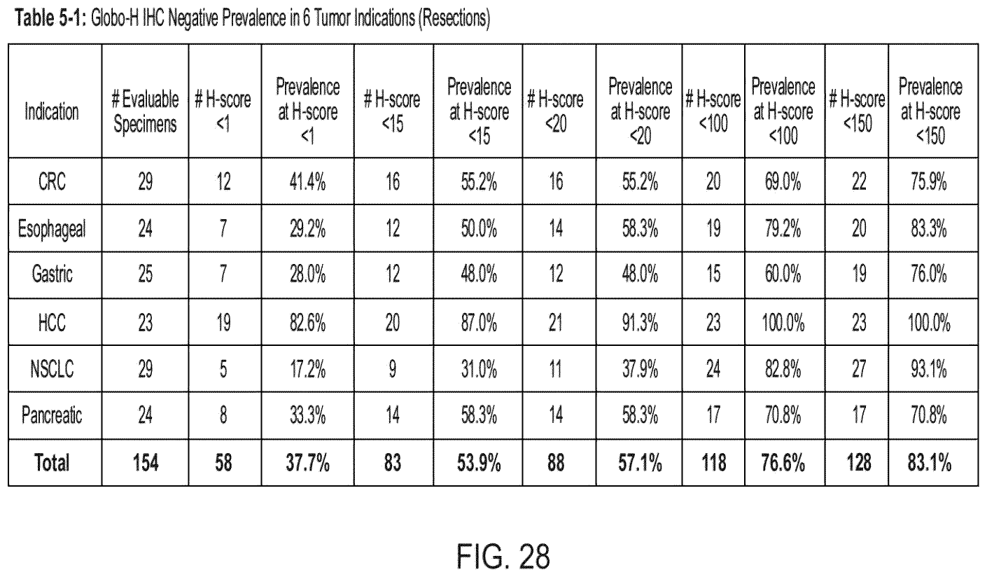

FIG. 28 shows Table 5-1.

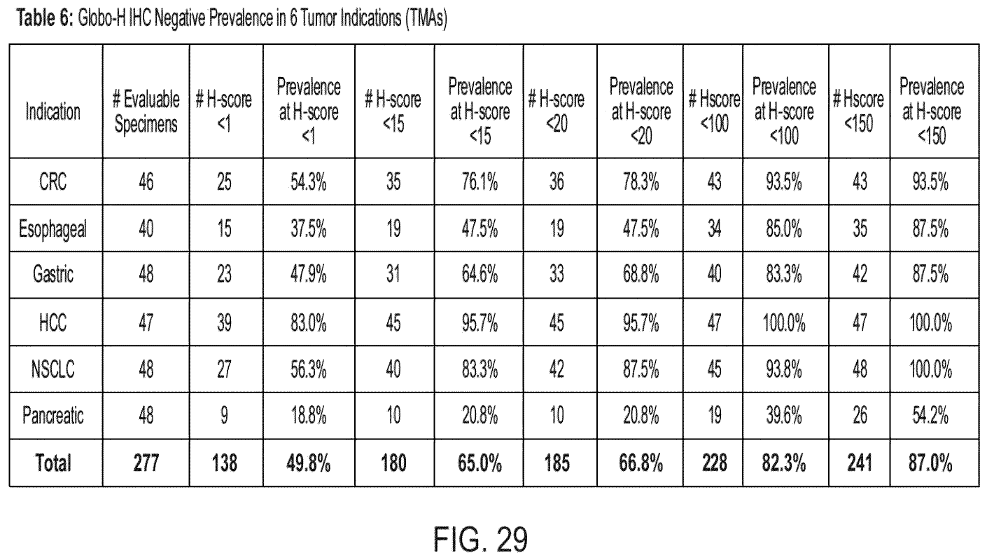

FIG. 29 shows Table 6.

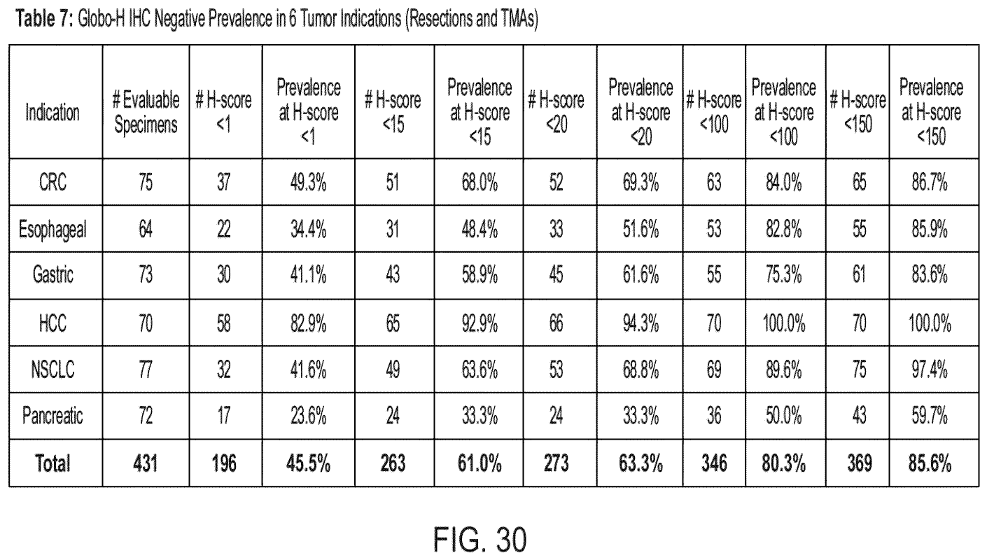

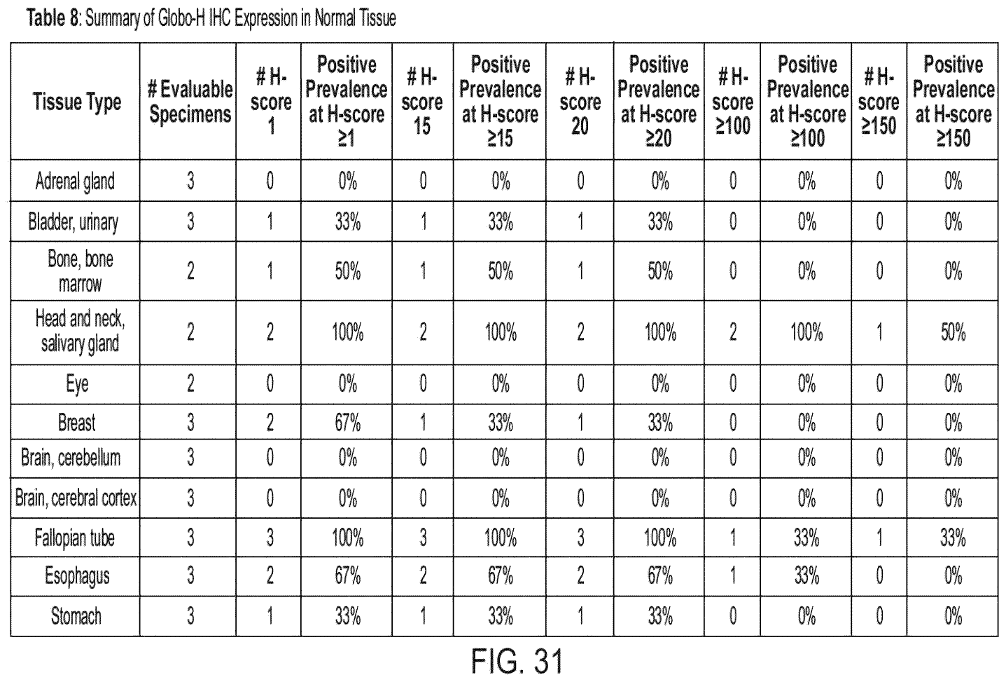

FIG. 30 shows Table 7.

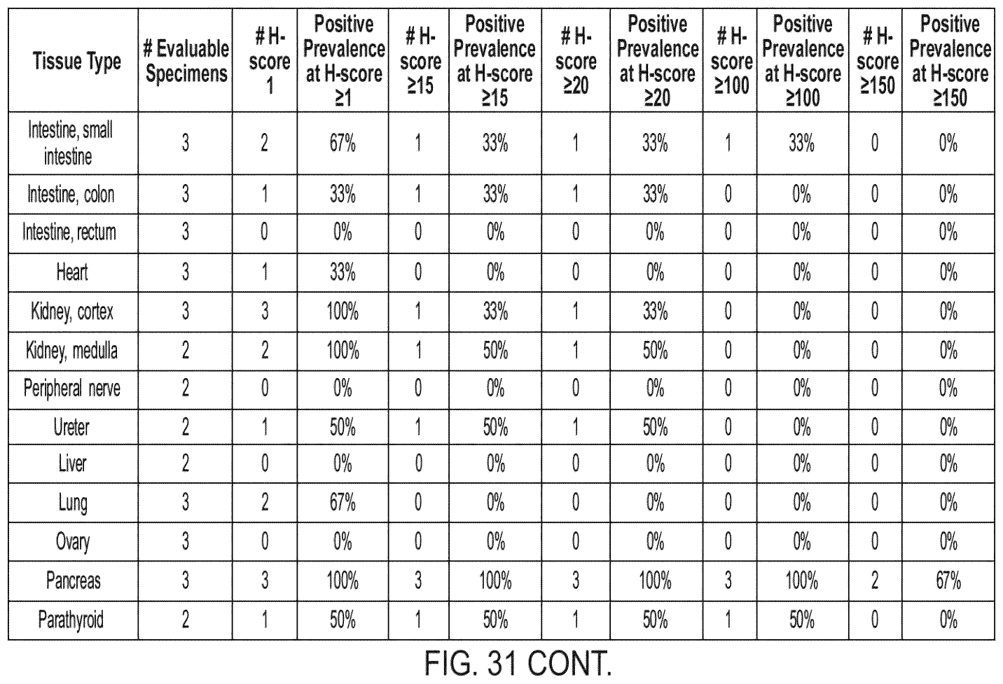

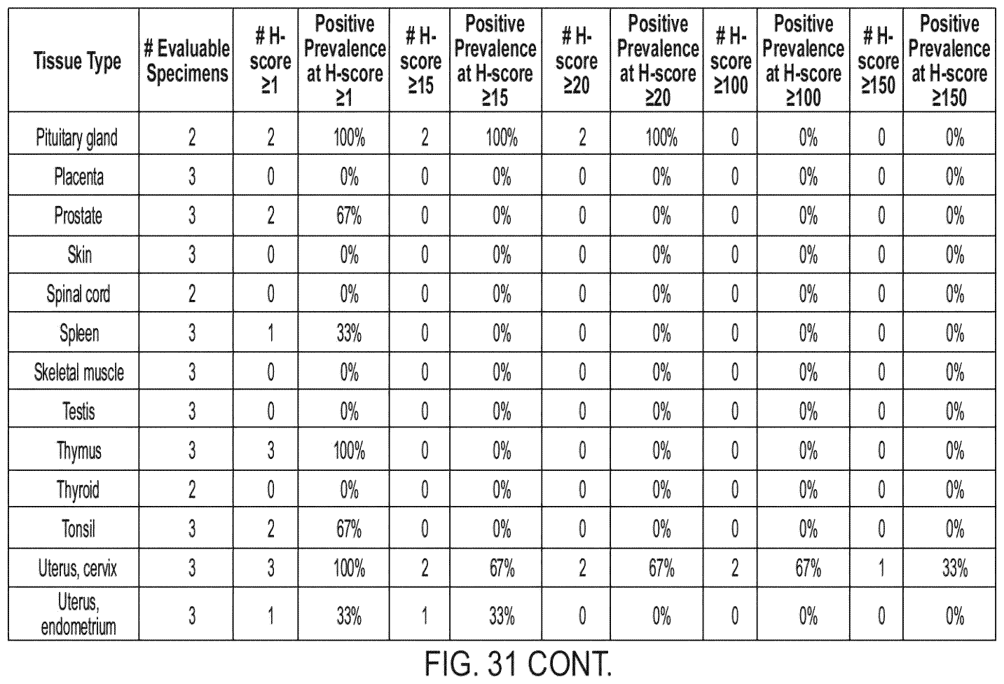

FIG. 31 shows Table 8.

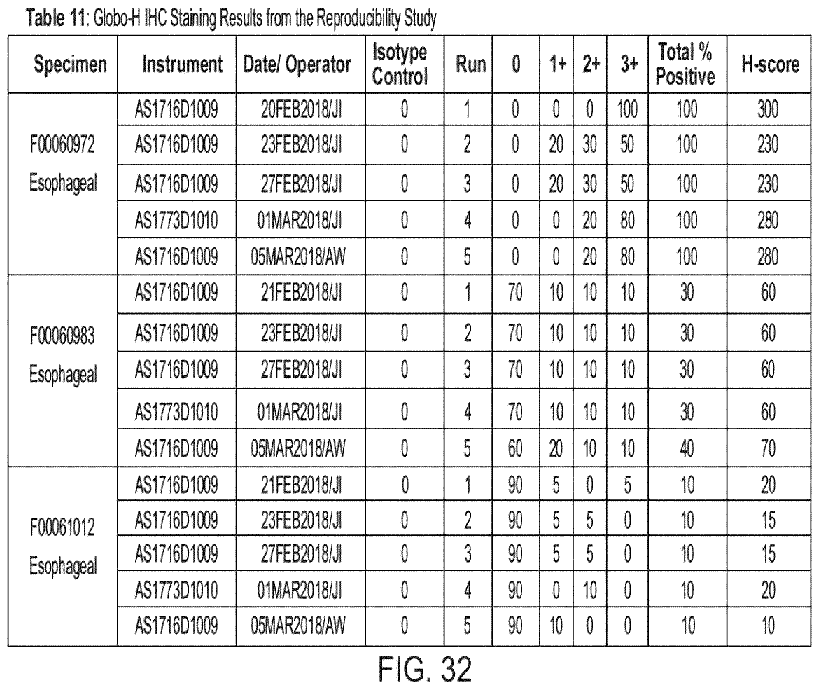

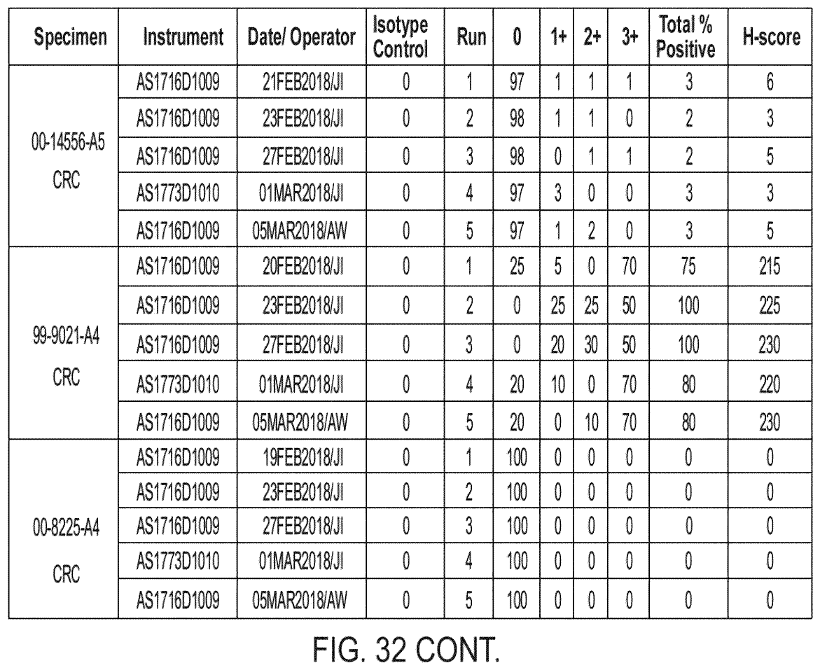

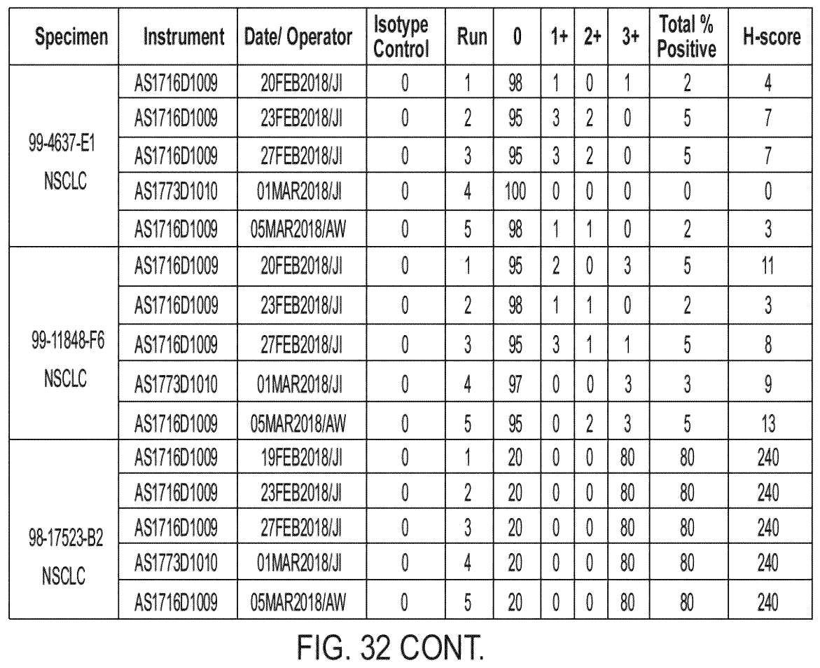

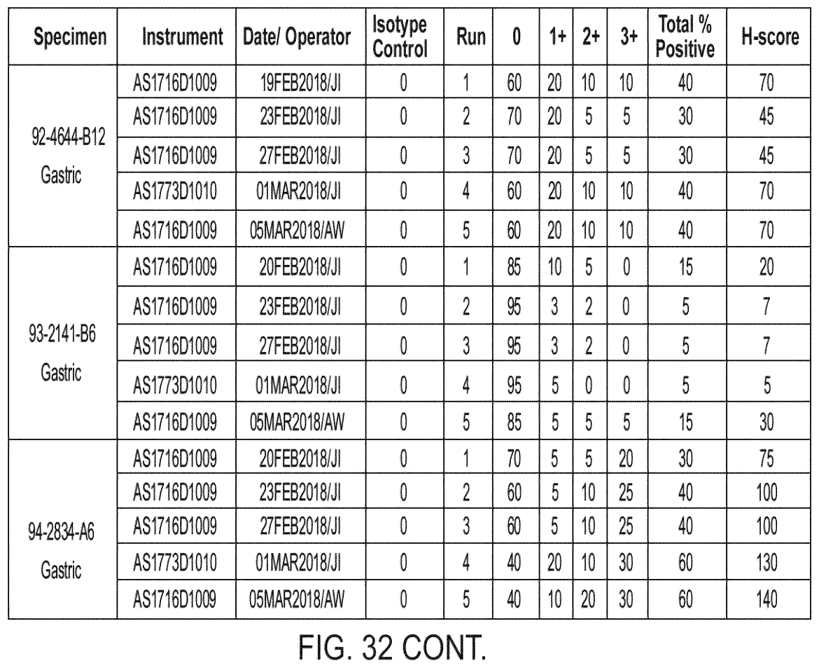

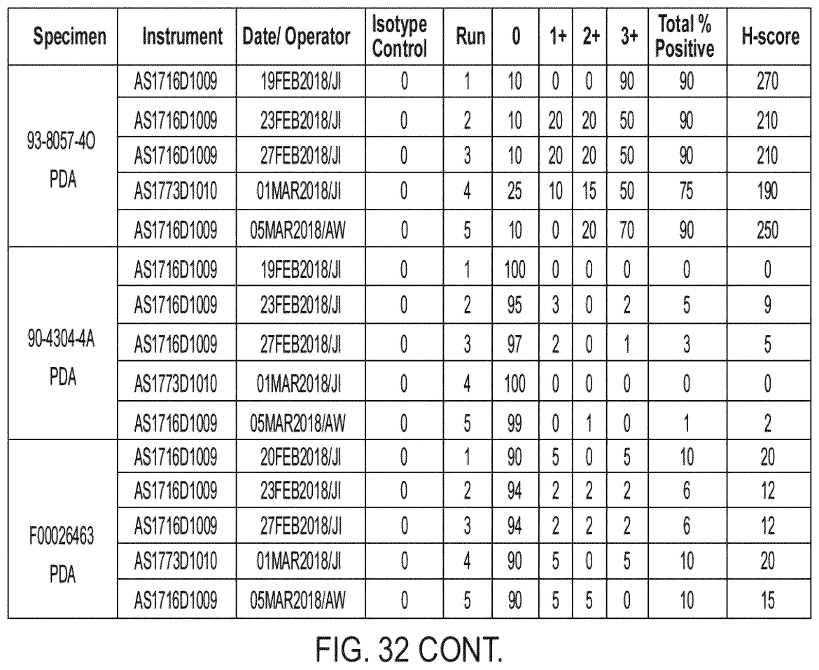

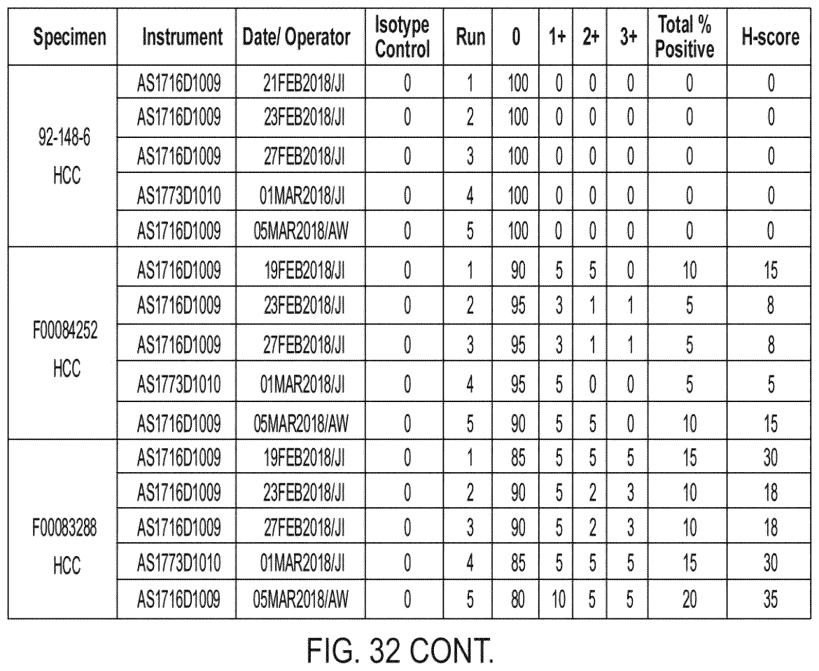

FIG. 32 shows Table 11.

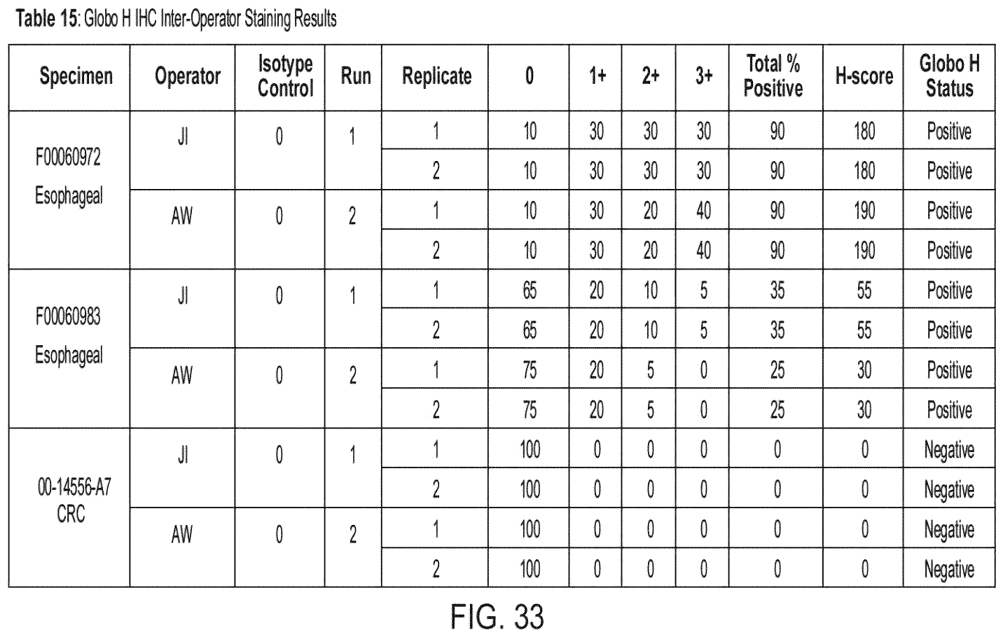

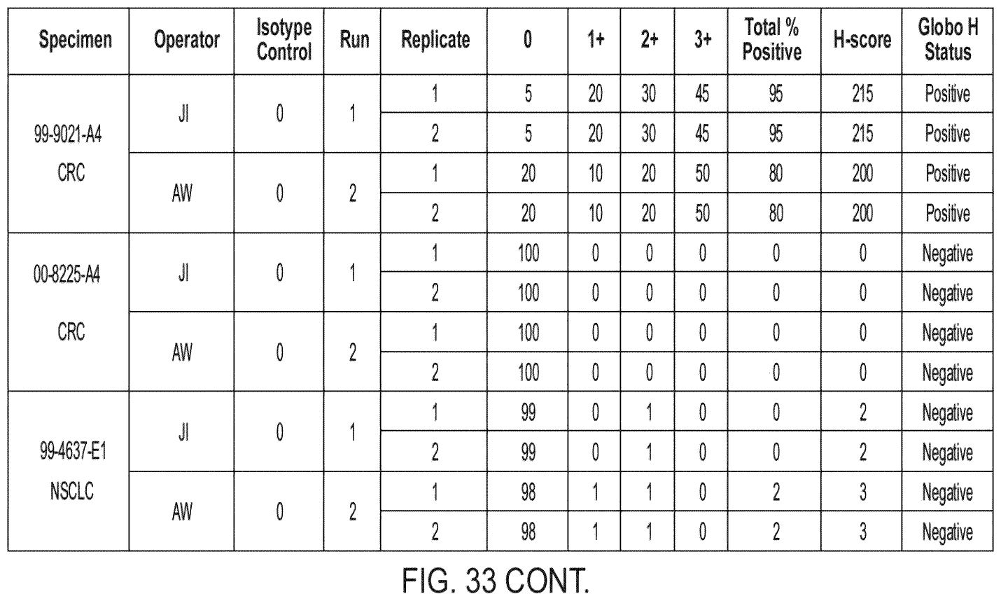

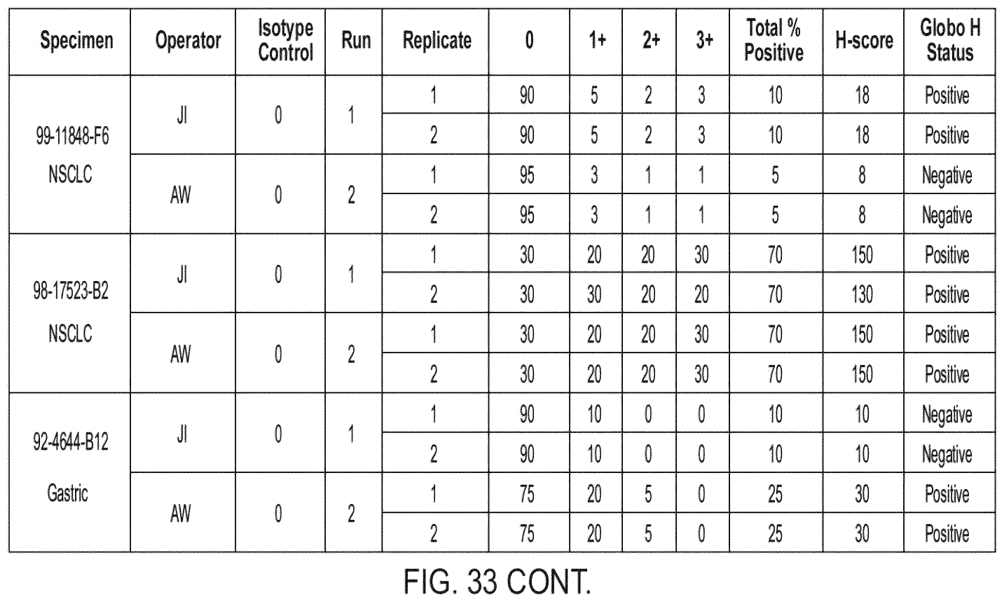

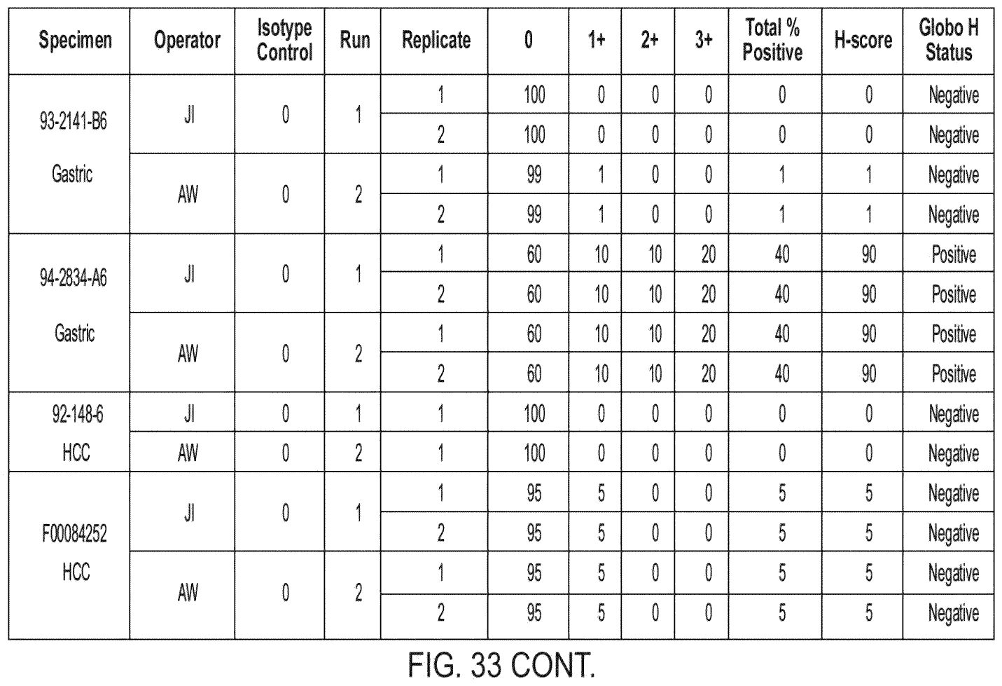

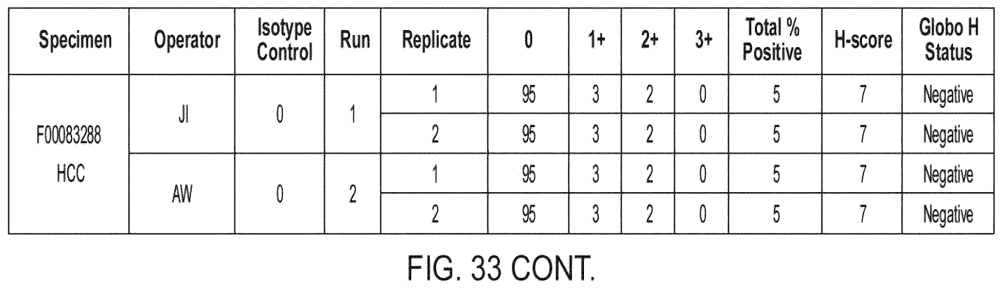

FIG. 33 shows Table 15.

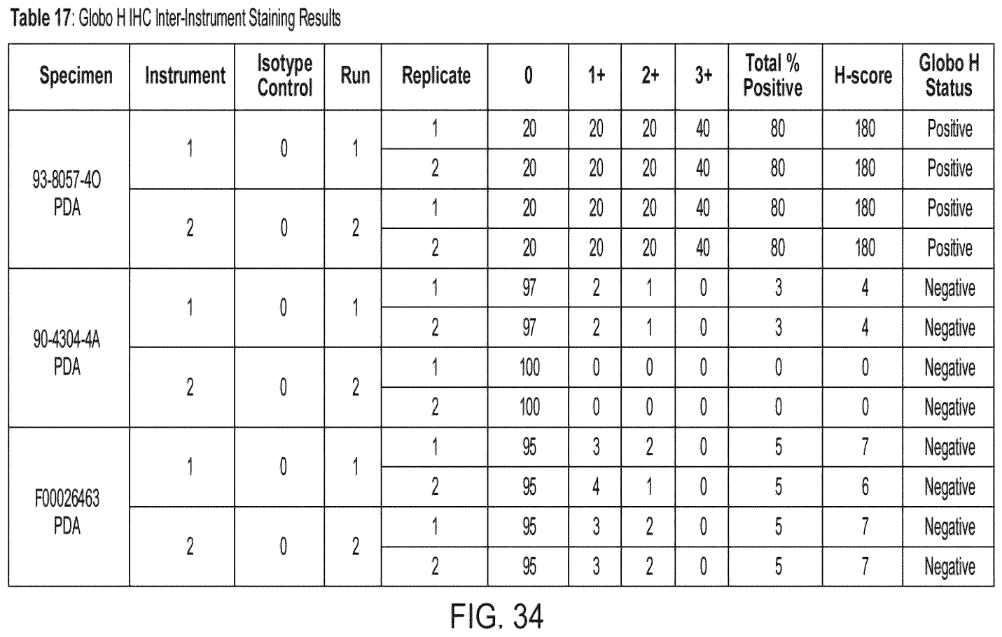

FIG. 34 shows Table 17.

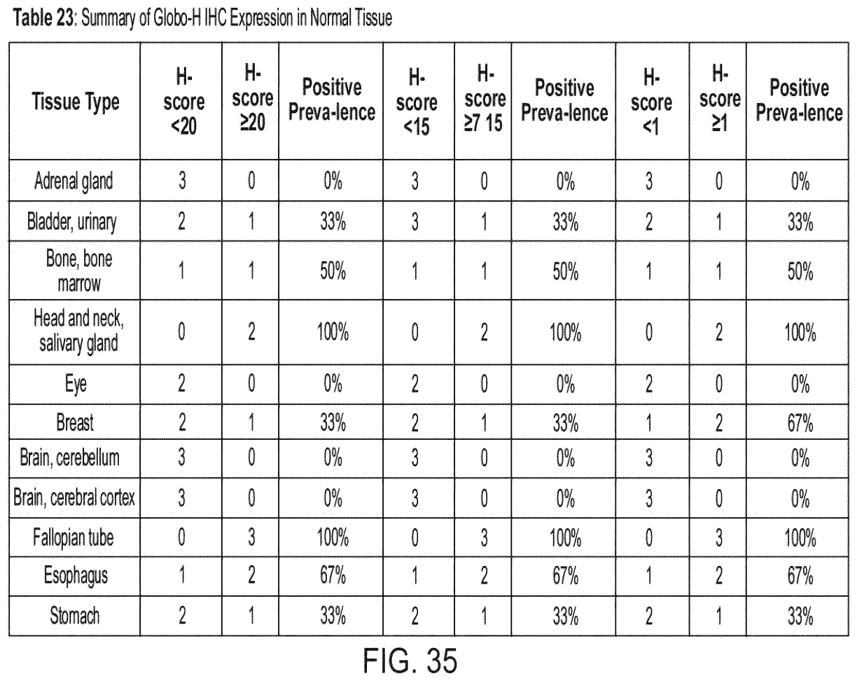

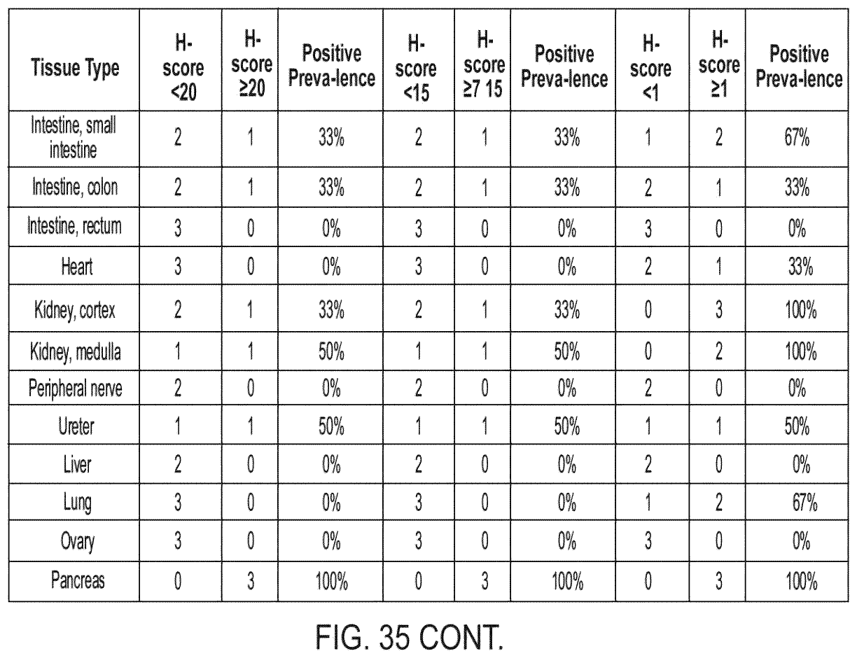

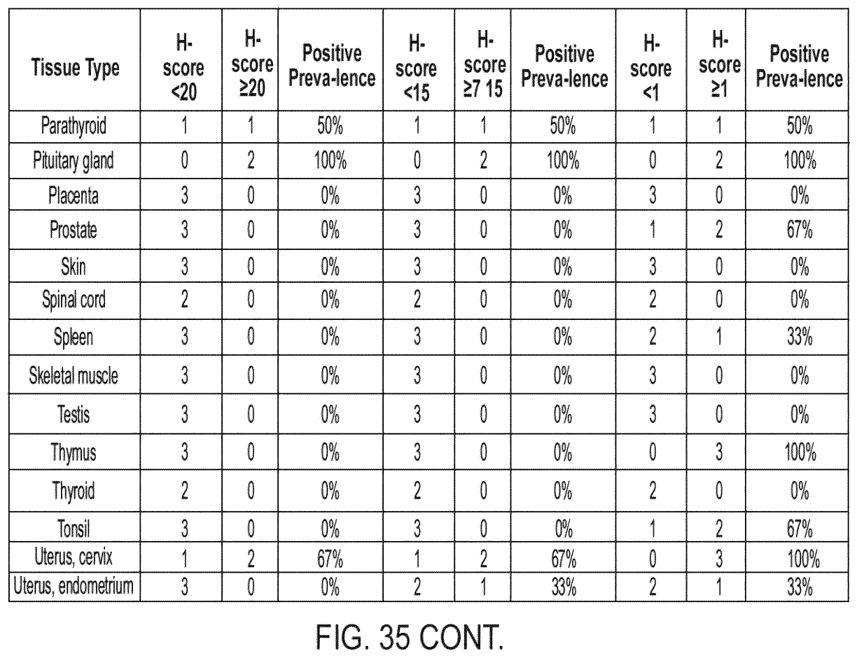

FIG. 35 shows Table 23.

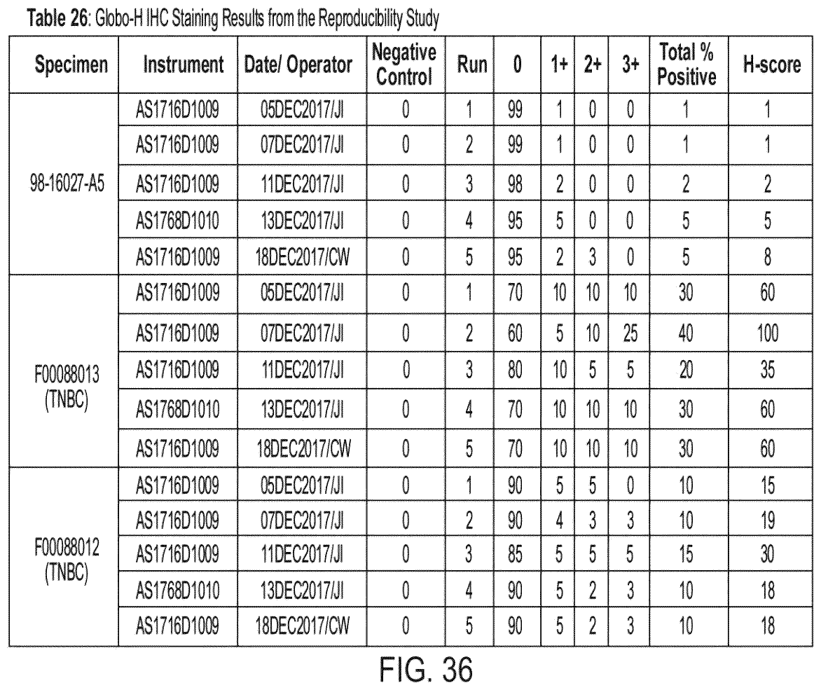

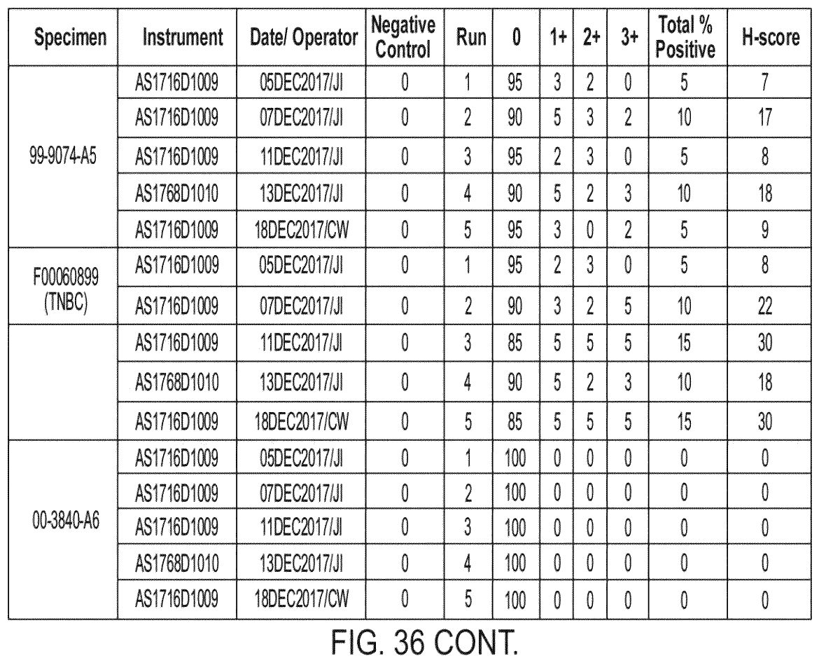

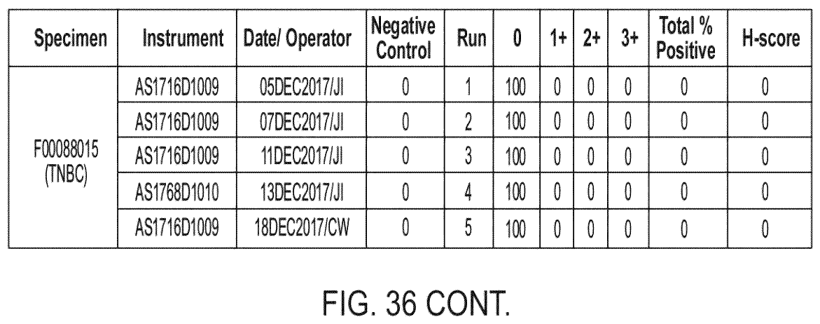

FIG. 36 shows Table 26.

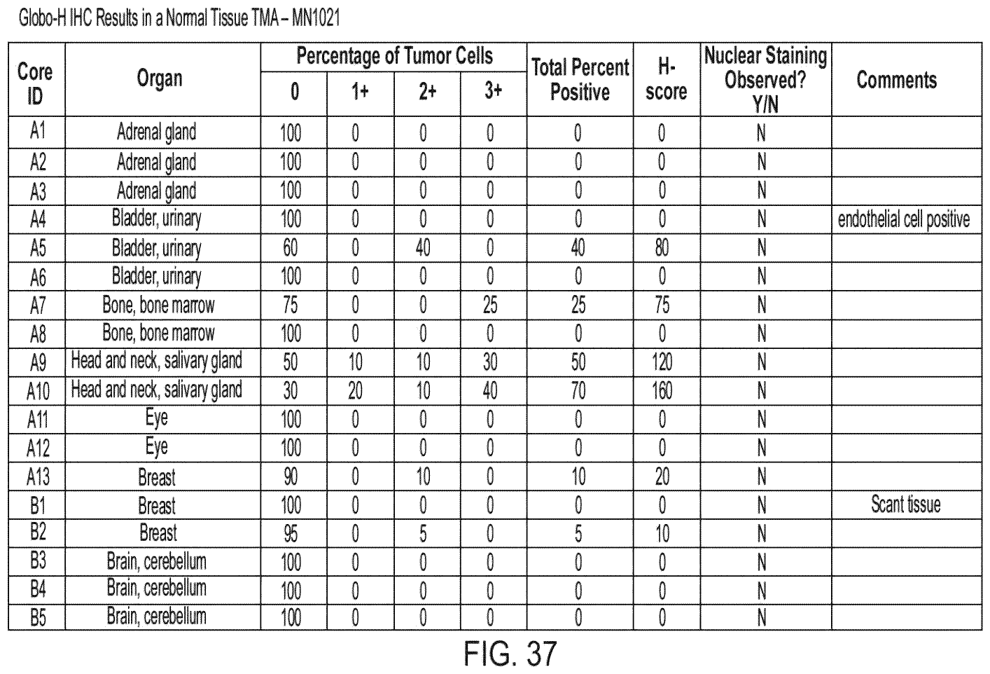

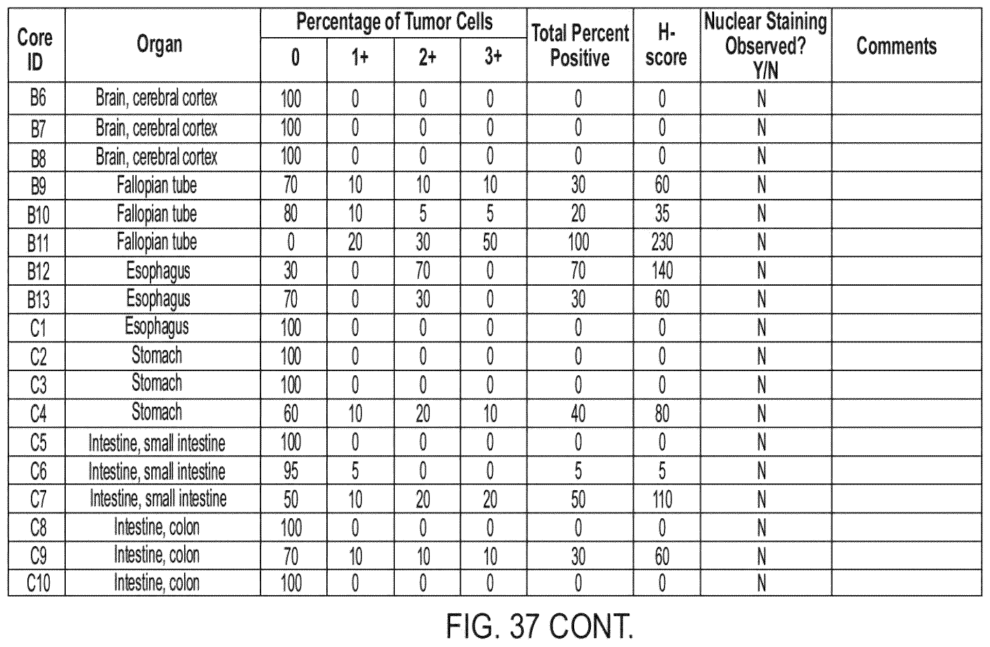

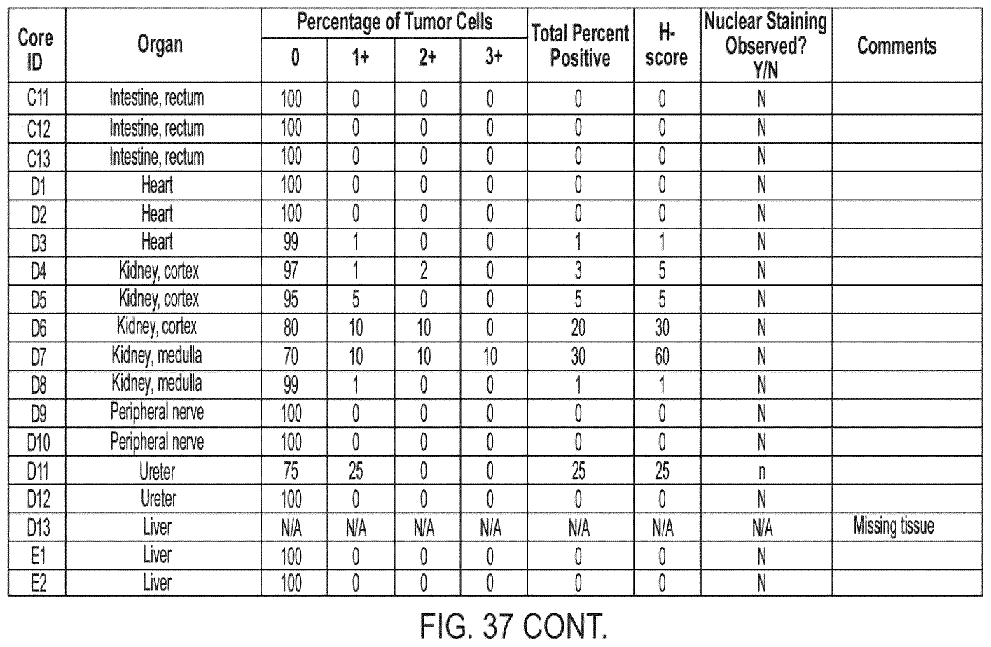

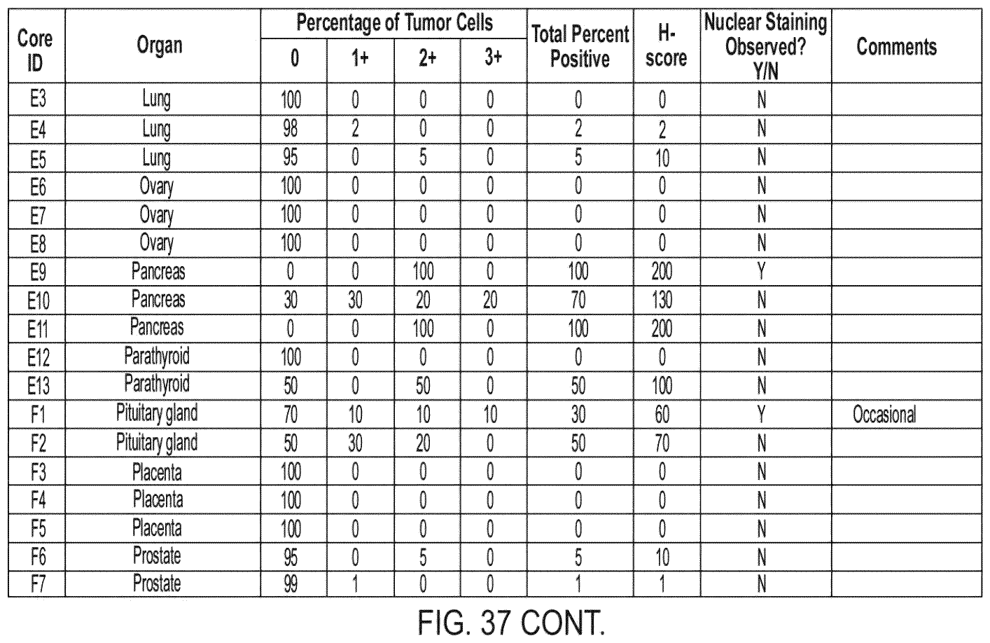

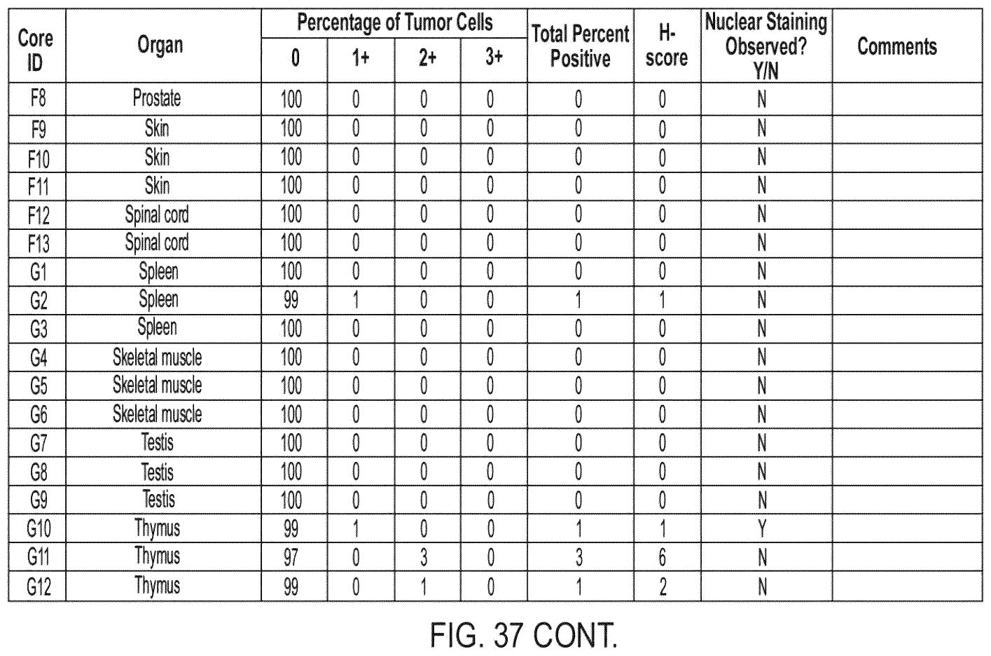

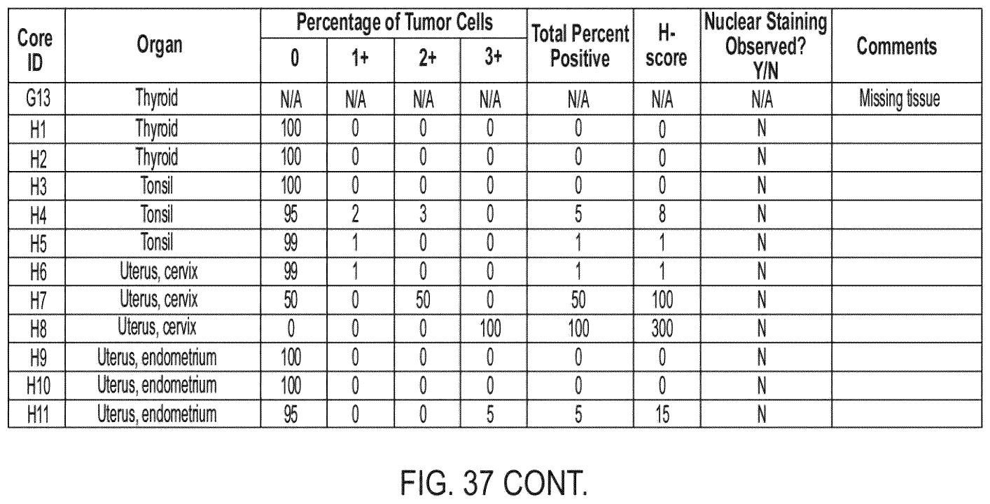

FIG. 37 shows Globo H IHC results in a normal tissue TMA-MN1201.

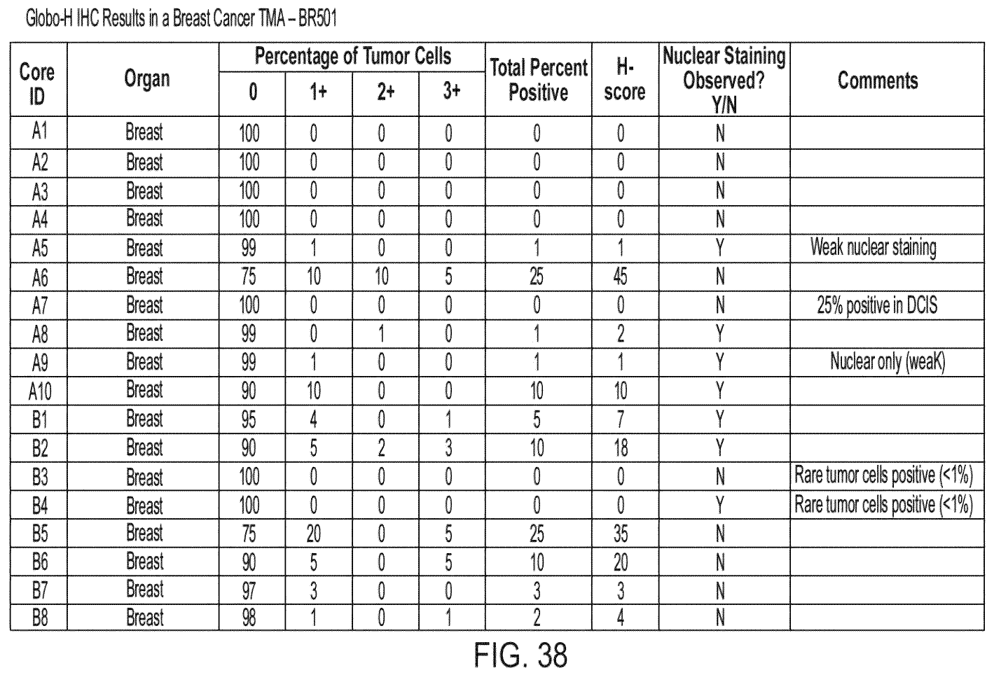

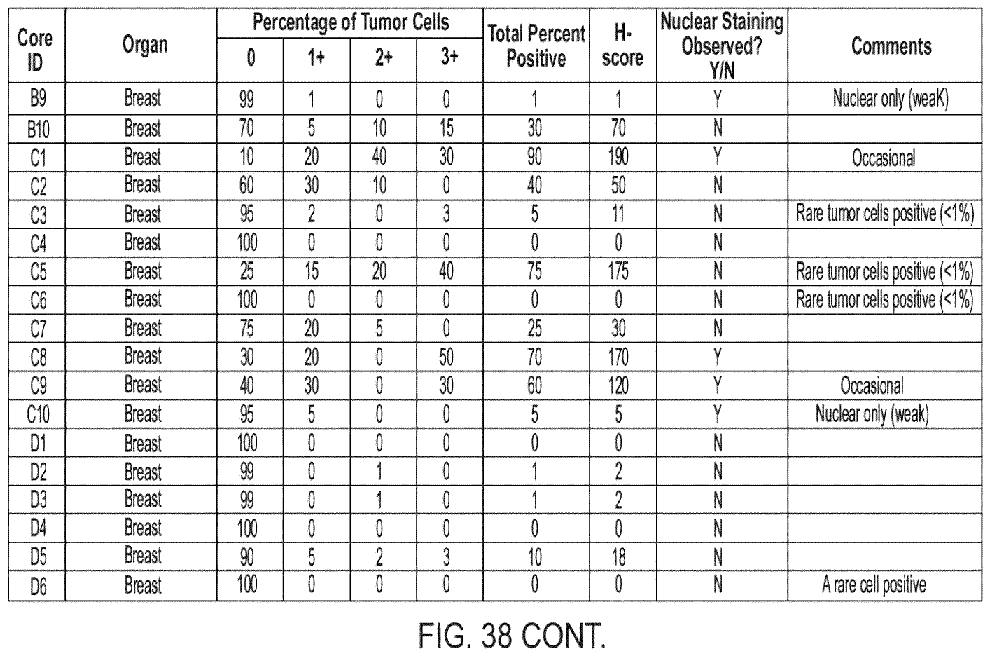

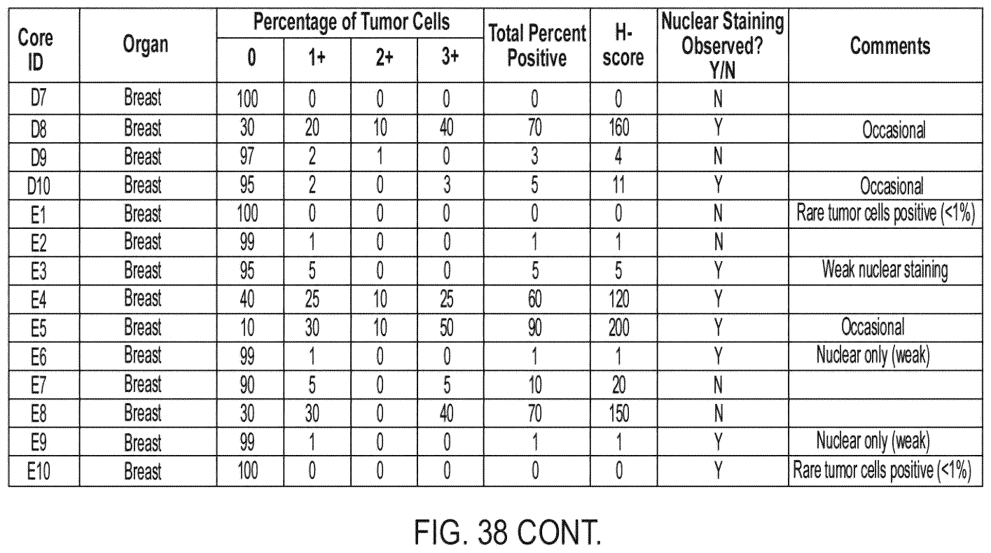

FIG. 38 shows Globo H IHC results in a normal tissue TMA-BR501.

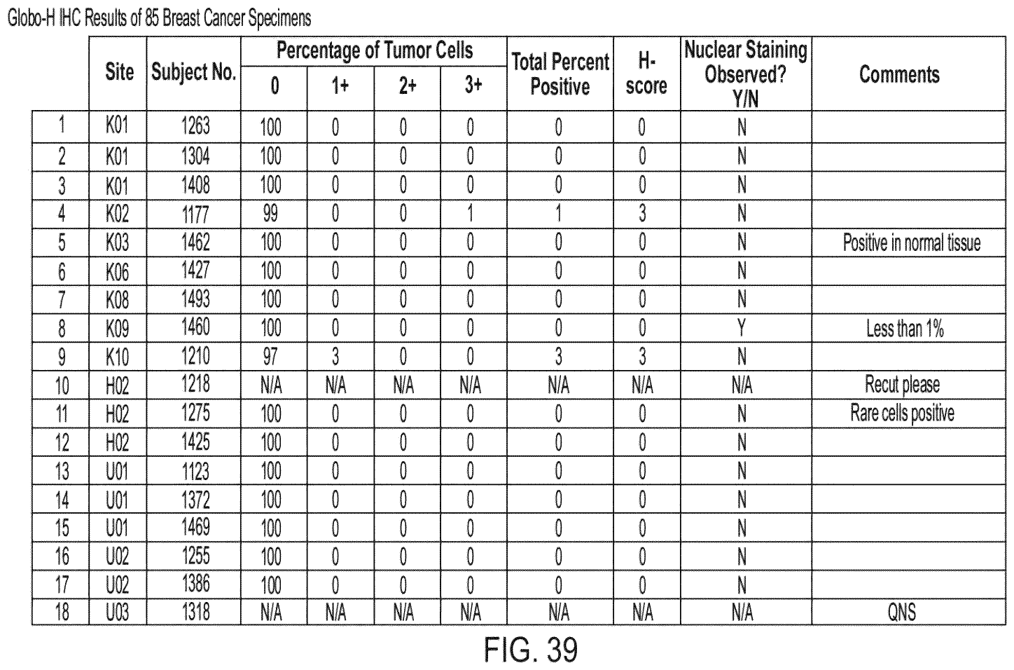

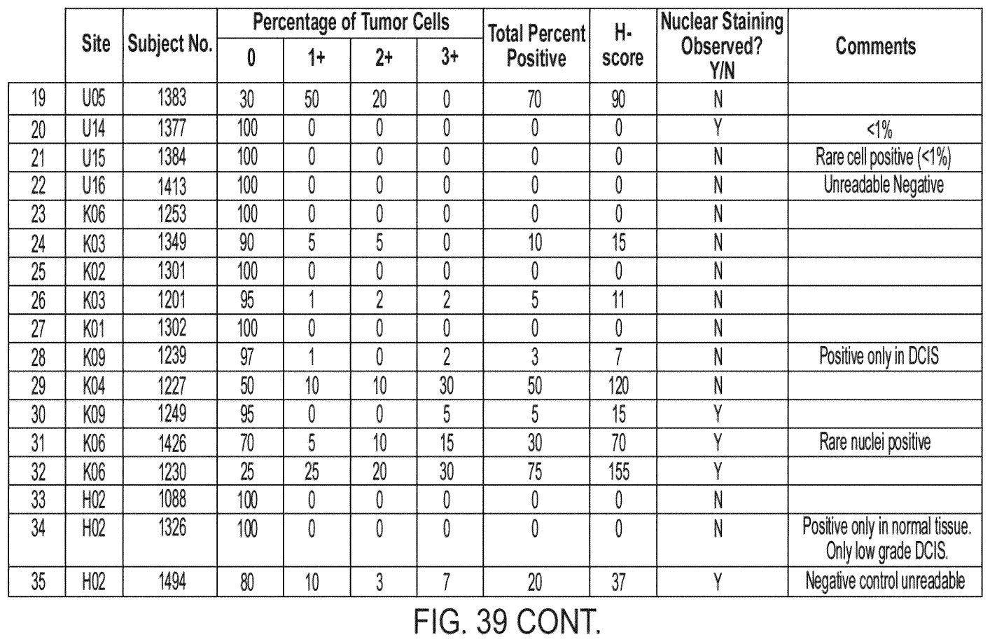

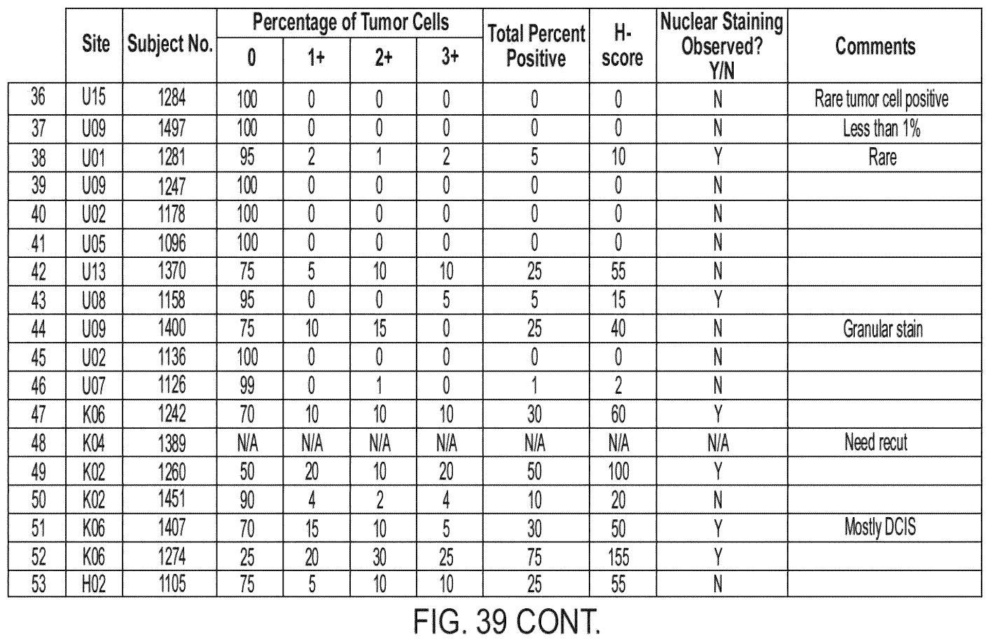

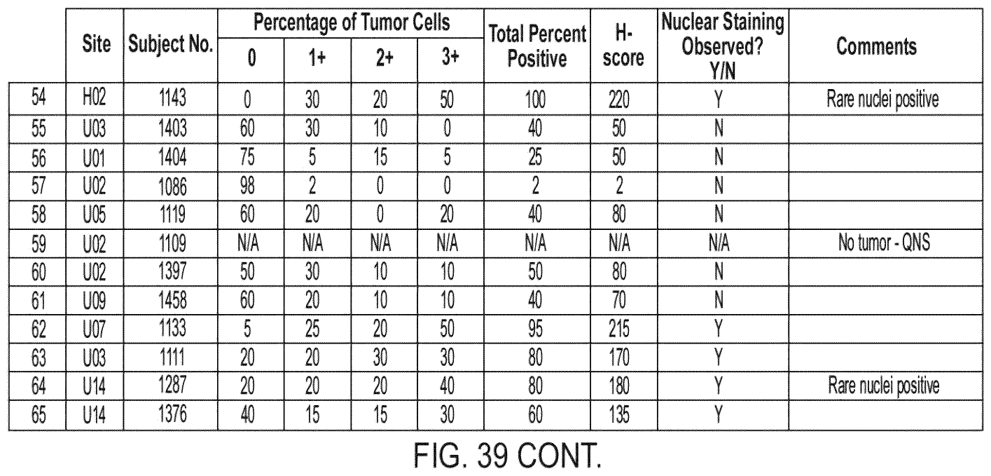

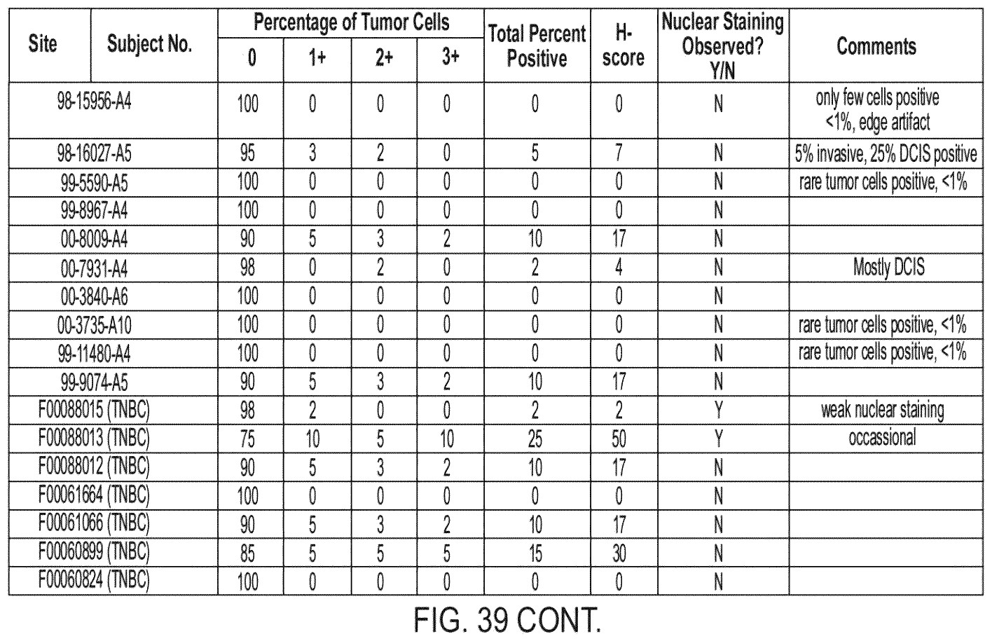

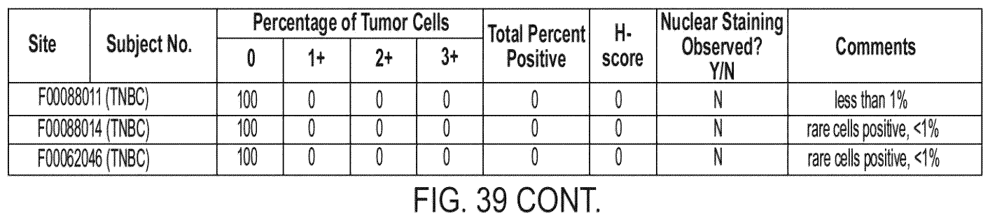

FIG. 39 shows Globo H IHC results of 85 breast cancer specimens.

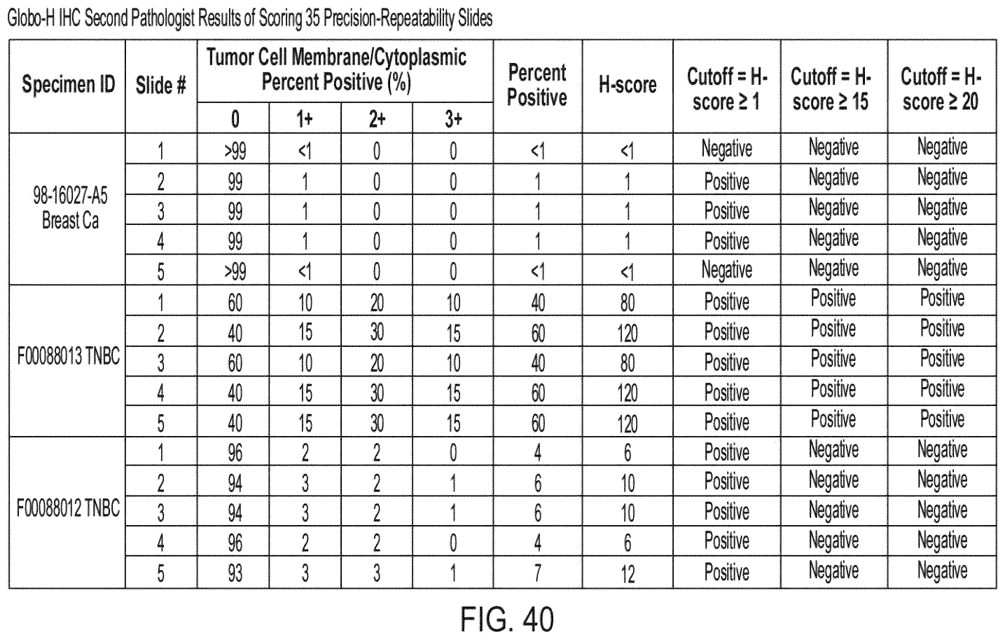

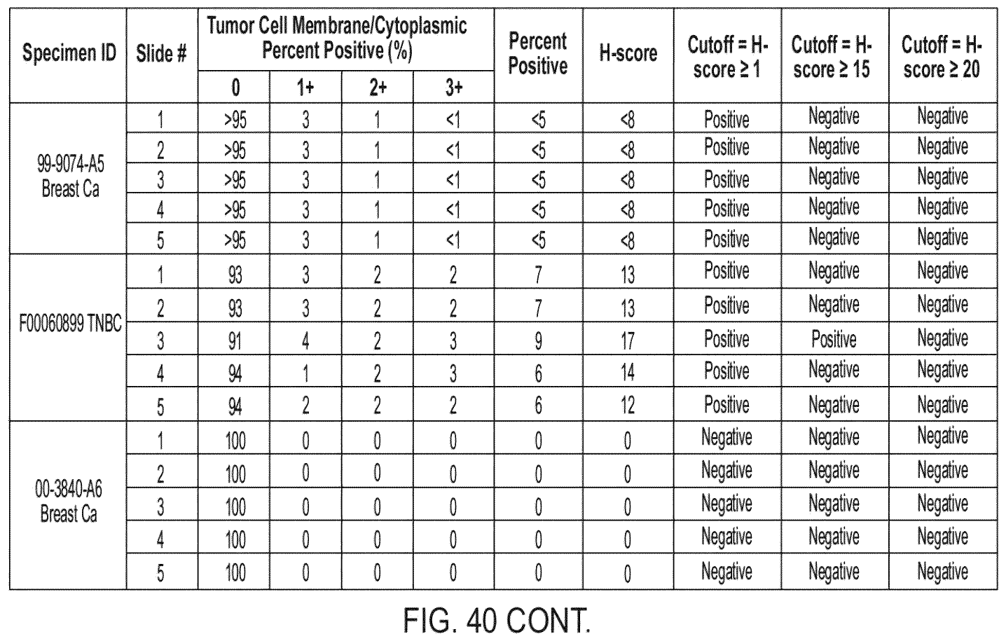

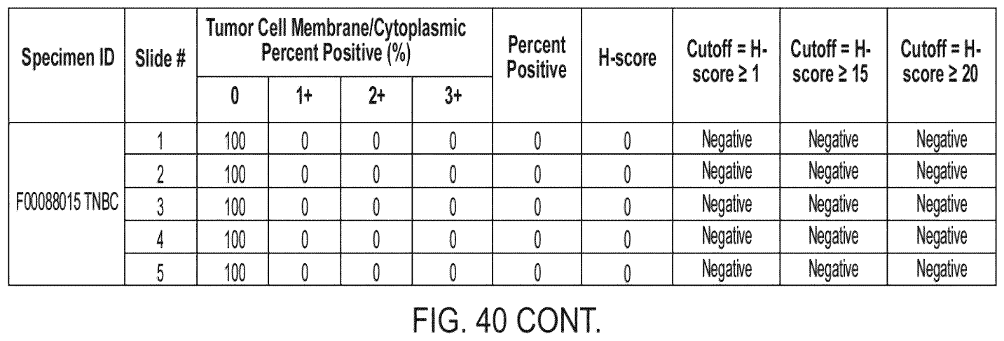

FIG. 40 shows Globo-H IHC second pathologies results of scoring 35 precision repeatability results slides.

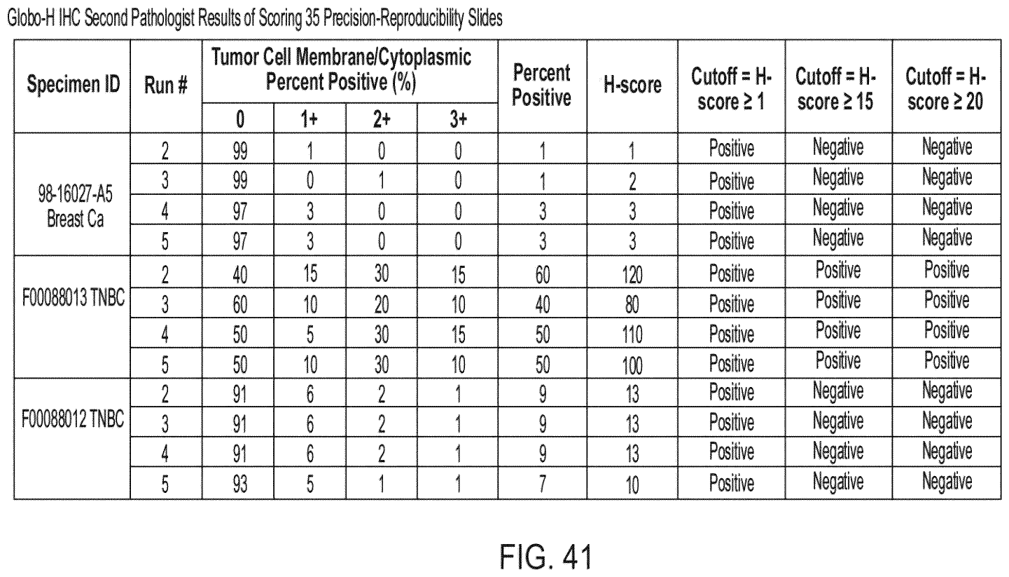

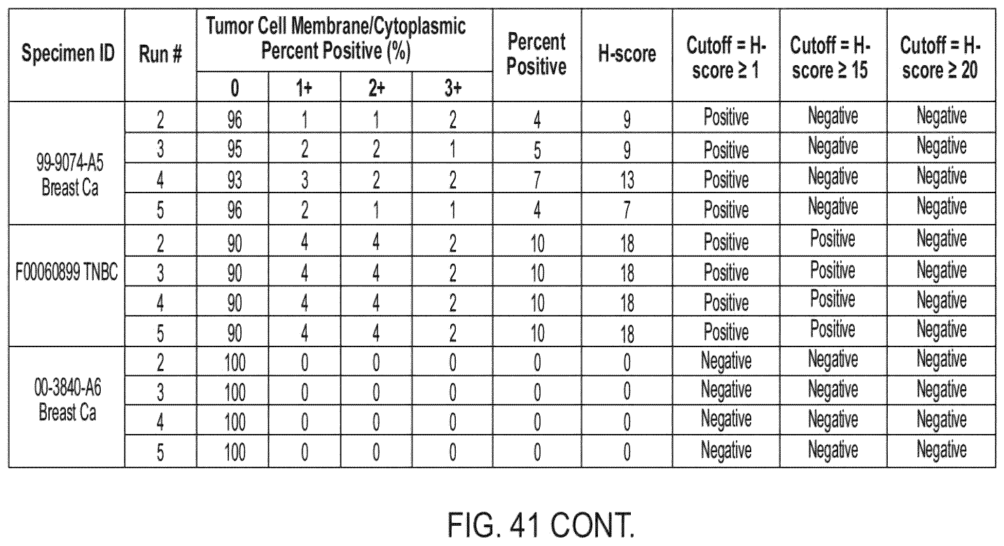

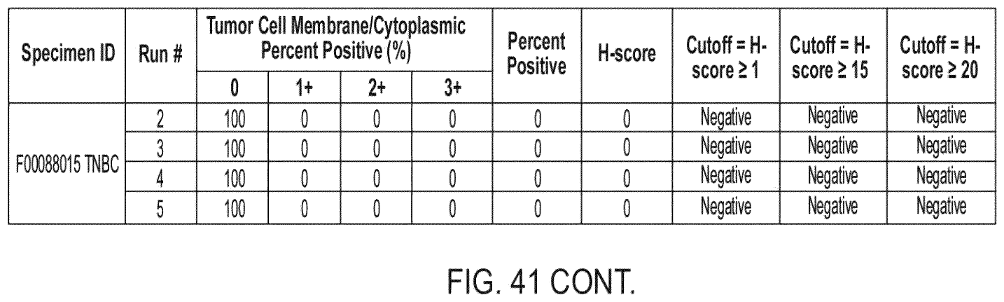

FIG. 41 shows Globo-H IHC second pathologies results of scoring 35 precision reproducibility results slides.

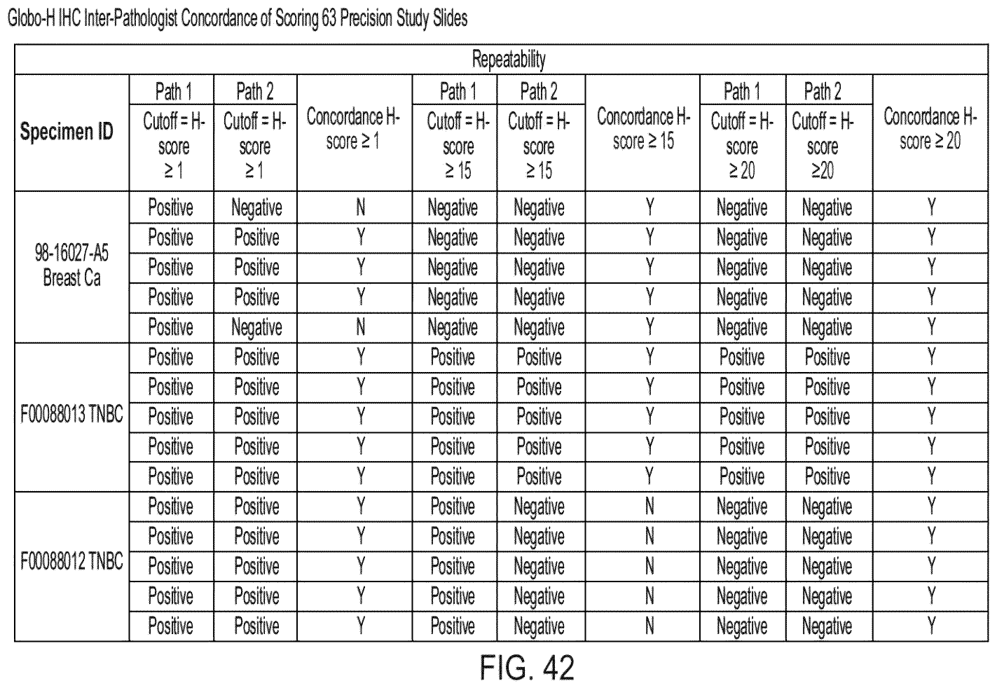

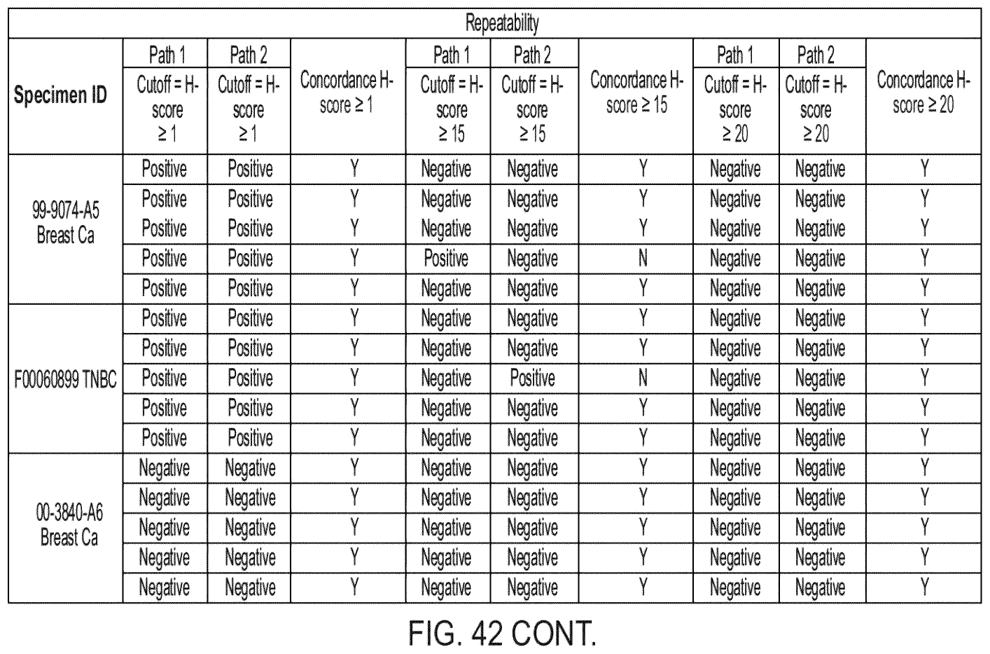

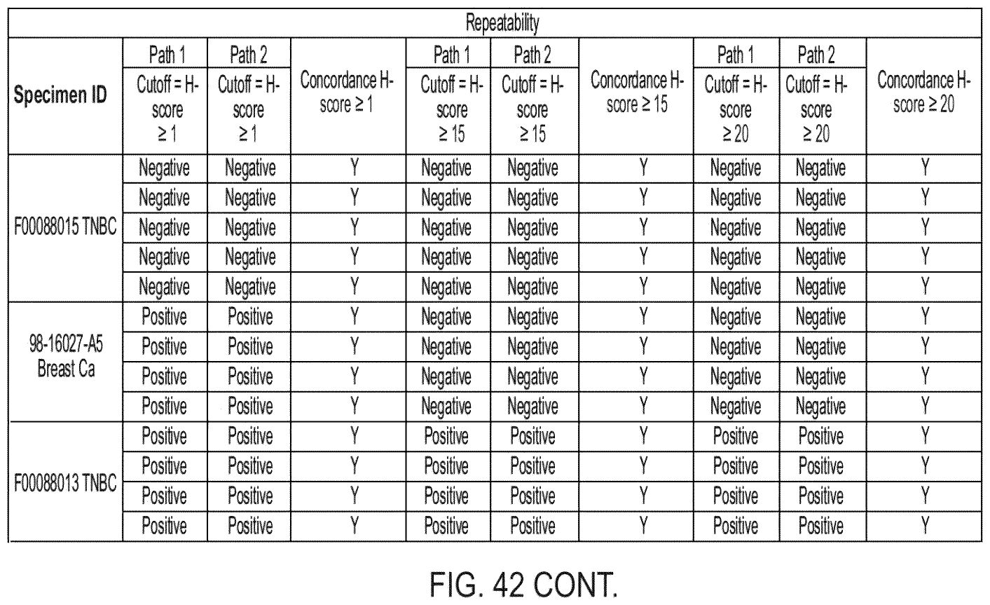

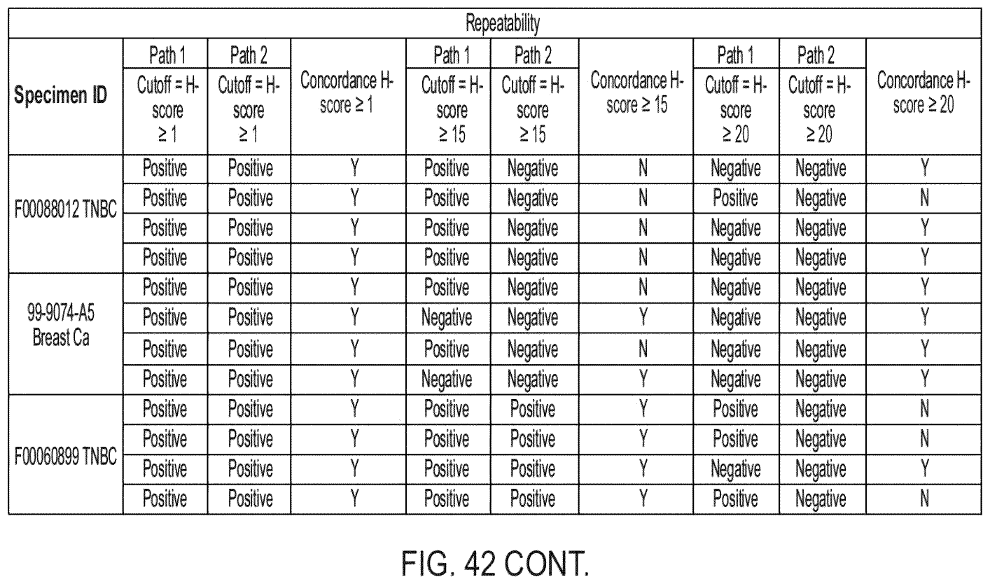

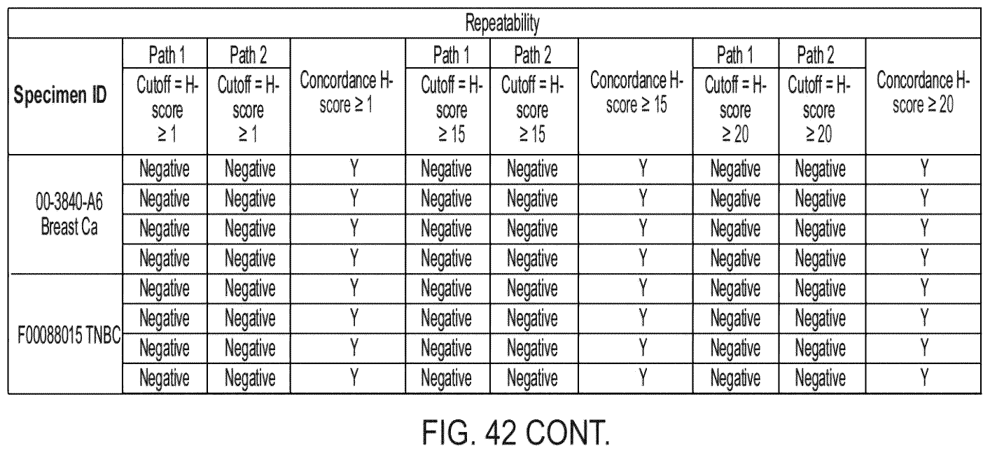

FIG. 42 shows Globo-H IHC inter pathologies concordance of scoring 63 precision study slides.

### DETAILED DESCRIPTION OF THE INVENTION

Accordingly, methods and compositions directed to the Globo-H biomarkers for use in diagnosing and treating a broad spectrum of cancers are provided.

### Definitions

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press, 1989); DNA Cloning, Volumes I and II (D. N. Glover ed., 1985); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Antibodies: A Laboratory Manual, by Harlow and Lane s (Cold Spring Harbor Laboratory Press, 1988); and Handbook Of Experimental Immunology, Volumes I-IV (D. M. Weir and C. C. Blackwell, eds., 1986).

As used herein, the term “glycan” refers to a polysaccharide, or oligosaccharide. Glycan is also used herein to refer to the carbohydrate portion of a glycoconjugate, such as a glycoprotein, glycolipid, glycopeptide, glycoproteome, peptidoglycan, lipopolysaccharide or a proteoglycan. Glycans usually consist solely of O-glycosidic linkages between monosaccharides. For example, cellulose is a glycan (or more specifically a glucan) composed of β-1,4-linked D-glucose, and chitin is a glycan composed of β-1,4-linked N-acetyl-D-glucosamine. Glycans can be homo or heteropolymers of monosaccharide residues, and can be linear or branched. Glycans can be found attached to proteins as in glycoproteins and proteoglycans. They are generally found on the exterior surface of cells. O- and N-linked glycans are very common in eukaryotes but may also be found, although less commonly, in prokaryotes. N-Linked glycans are found attached to the R-group nitrogen (N) of asparagine in the sequon. The sequon is a Asn-X-Ser or Asn-X-Thr sequence, where X is any amino acid except praline.

As used herein, the term “level of expression” when referring to Globo-H levels refers to the measurable quantity of a given carbohydrate antigen as determined by IHC and/or hybridization measurements and which corresponds in direct proportion with the extent to which the carbohydrate antigen is expressed. The level of expression of a carbohydrate antigen is determined by methods known in the art.

The term “label” refers to a composition capable of producing a detectable signal indicative of the presence of the labeled molecule. Suitable labels include HRP, radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

As used herein, the term “predetermined level” refers generally at an assay cutoff value that is used to assess diagnostic results by comparing the assay results against the predetermined level, and where the predetermined level already that has been linked or associated with various clinical parameters (e.g., monitoring whether a subject being treated with a drug has achieved an efficacious blood level of the drug, monitoring the response of a subject receiving treatment for cancer with an anti-cancer drug, monitoring the response of a tumor in a subject receiving treatment for said tumor, etc.). The predetermined level may be either an absolute value or a value normalized by subtracting the value obtained from a patient prior to the initiation of therapy. An example of a predetermined level that can be used is a baseline level obtained from one or more subjects that may optionally be suffering from one or more diseases or conditions.

The term “support” refers to conventional supports such as beads, particles, dipsticks, fibers, filters, membranes and silane or silicate supports such as glass slides.

The invention comprises diagnostic assays performed on a patient tissue sample of any type or a derivate thereof, including peripheral blood, tumor or suspected tumor tissues (including fresh frozen and fixed or paraffin embedded tissue), cell isolates such as circulating epithelial cells separated or identified in a blood sample. Lymph node tissue, bone marrow and fine needle aspirates. Preferred tissue samples for use herein are peripheral blood, tumor or suspected tumor tissue and bone marrow.

### Assays

The inventive assays include assays both to select patients eligible to receive anti-Globo H therapy and assays to monitor patient response. Assays for response prediction are run before therapy selection and patients with elevated levels are eligible to receive anti-Globo H therapy. For monitoring patient response, the assay is run at the initiation of therapy to establish baseline (or predetermined) levels of the biomarker in the tissue sample. The same tissue is then sampled and assayed and the levels of the biomarker compared to the baseline or predetermined levels. The comparison (or informational analysis) of the level of the assayed biomarker with the baseline or predetermined level can be done by an automated system, such as a software program or intelligence system that is part of, or compatible with, the equipment (e.g., computer platform) on which the assay is carried out. Alternatively, this comparison or informational analysis can be done by a physician. In those instances where the levels remain the same or decrease, the therapy is likely being effective and can be continued. Where significant increase over baseline level (or predetermined level) occurs, the patient may not be responding.

The assays of the present invention can be performed by protein assay methods. Any type of either protein assays can be used. Protein assay methods useful in the invention are known in the art and comprise (i) immunoassay methods involving binding of a labeled antibody or protein to the expressed glycan marker, (ii) quantitative or qualitative colorimetric methods to determine expressed glycan markers or (iii) glycan array chip assays. Useful immunoassay methods include both solution phase assays conducted using any format known in the art, such as, but not limited to, an ELISA format, a sandwich format, a competitive inhibition format (including both forward or reverse competitive inhibition assays) or a fluorescence polarization format, and solid phase assays such as immunohistochemistry (referred to as “IHC”).

IHC methods are particularly preferred assays. IHC is a method of detecting the presence of specific moiety in cells or tissues and consists of the following steps: 1) a slide is prepared with the tissue to be interrogated; 2) a primary antibody is applied to the slide and binds to specific antigen; 2) the resulting antibody-antigen complex is bound by a secondary, enzyme-conjugated, antibody; 3) in the presence of substrate and chromogen, the enzyme forms a colored deposit (a “stain”) at the sites of antibody-antigen binding; and 4) the slide is examined under a microscope to identify the presence of and extent of the stain.

### Sample Processing and Assay Performance

The tissue sample to be assayed by the inventive methods can comprise any type, including a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample. For example, a patient peripheral blood sample can be initially processed to extract an epithelial cell population, and this extract can then be assayed. A microdissection of the tissue sample to obtain a cellular sample enriched with suspected tumor cells can also be used. The preferred tissue samples for use herein are peripheral blood, tumor tissue or suspected tumor tissue, including fine needle aspirates, fresh frozen tissue and paraffin embedded tissue, and bone marrow.

The tissue sample can be processed by any desirable method for performing IHC (immunohistochemistry), in situ hybridization or other protein assays. For the preferred in situ hybridization assays, a paraffin embedded tumor tissue sample or bone marrow sample is fixed on a glass microscope slide and deparaffinized with a solvent, typically xylene. Useful protocols for tissue deparaffinization and in situ hybridization are available from commercial sources. Any suitable instrumentation or automation can be used in the performance of the inventive assays. Automated imaging can be employed for the preferred IHC in situ hybridization assays.

In one embodiment, the sample comprises a peripheral blood sample from a patient which is processed to produce an extract of circulating tumor cells having increased expression of the glycan marker. The circulating tumor cells can be separated by immunomagnetic separation technology. The number of circulating tumor cells showing altered expression of glycan marker is then compared to the baseline level of circulating tumor cells having altered expression of glycan marker determined preferably at the start of therapy.

Test samples can comprise any number of cells that is sufficient for a clinical diagnosis, and typically contain at least about 100 cells.

In another aspect, the invention comprises immunoassay kits for the detection of which kits comprise a labeled antibody. These kits may also include an antibody capture reagent or antibody indicator reagent useful to carry out a sandwich immunoassay. Exemplary kits of the invention comprise containers containing, respectively, at least one antibody capable of binding specifically to at least one of the glycan markers in the set, and a control protein. Any suitable control composition for the particular glycan marker assay can be included in the kits of the invention. The control compositions generally comprise the glycan marker to be assayed for, along with any desirable additives. One or more additional containers may enclose elements, such as reagents or buffers, to be used in the assay. Such kits may also, or alternatively, contain a detection reagent as described above that contains a reporter group suitable for direct or indirect detection of antibody binding. In certain embodiments, the kit comprises instructions for use which can further comprise guidelines for tumor staining scoring and guidelines for clinical interpretation.

As used herein, the term “antigen” is defined as any substance capable of eliciting an immune response.

As used herein, the term “immunogenicity” refers to the ability of an immunogen, antigen, or vaccine to stimulate an immune response.

As used herein, the term “epitope” is defined as the parts of an antigen molecule which contact the antigen binding site of an antibody or a T cell receptor.

As used herein, the term “vaccine” refers to a preparation that contains an antigen, consisting of whole disease-causing organisms (killed or weakened) or components of such organisms, such as proteins, peptides, or polysaccharides, that is used to confer immunity against the disease that the organisms cause. Vaccine preparations can be natural, synthetic or derived by recombinant DNA technology.

As used herein, the term “antigen specific” refers to a property of a cell population such that supply of a particular antigen, or a fragment of the antigen, results in specific cell proliferation.

As used herein, the term “specifically binding,” refers to the interaction between binding pairs (e.g., an antibody and an antigen). In various instances, specifically binding can be embodied by an affinity constant of about 10⁻⁶ moles/liter, about 10⁻⁷ moles/liter, or about 10⁻⁸ moles/liter, or less.

The phrase “substantially similar,” “substantially the same”, “equivalent”, or “substantially equivalent”, as used herein, denotes a sufficiently high degree of similarity between two numeric values (for example, one associated with a molecule and the other associated with a reference/comparator molecule) such that one of skill in the art would consider the difference between the two values to be of little or no biological and/or statistical significance within the context of the biological characteristic measured by said values (e.g., Kd values, anti-viral effects, etc.). The difference between said two values is, for example, less than about 50%, less than about 40%, less than about 30%, less than about 20%, and/or less than about 10% as a function of the value for the reference/comparator molecule.

The phrase “substantially reduced,” or “substantially different”, as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

The terms “antibody” and “immunoglobulin” are used interchangeably in the broadest sense and include monoclonal antibodies (e.g., full length or intact monoclonal antibodies), polyclonal antibodies, monovalent, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

The “variable region” or “variable domain” of an antibody refers to the amino-terminal domains of heavy or light chain of the antibody. These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (2000). An antibody may be part of a larger fusion molecule, formed by covalent or non-covalent association of the antibody with one or more other proteins or peptides.

The terms “full length antibody,” “intact antibody” and “whole antibody” are used herein interchangeably, to refer to an antibody in its substantially intact form, not antibody fragments as defined below. The terms particularly refer to an antibody with heavy chains that contain the Fc region.

“Antibody fragments” comprise only a portion of an intact antibody, wherein the portion retains at least one, and as many as most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise an antigen binding arm linked to an Fc sequence capable of conferring in vivo stability to the fragment.

The term “monoclonal antibody” as used herein refers to an antibody obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell hybridomas 563-681 (Elsevier, N.Y., 1981)), recombinant DNA methods (see, e.g., U.S. Pat. No. 4,816,567), phage display technologies (See, e.g., Clackson et al., Nature, 352:624-628 (1991); Marks et al., J. Mol. Biol. 222:581-597 (1992); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004), and technologies for producing human or human-like antibodies in animals that have parts or all of the human immunoglobulin loci or genes encoding human immunoglobulin sequences (see, e.g., WO98/24893; WO96/34096; WO96/33735; WO91/10741; Jakobovits et al., Proc. Natl. Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; 5,661,016; Marks et al., Bio. Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996) and Lonberg and Huszar, Intern. Rev. Immunol. 13:65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

Antibodies of the present invention also include chimerized or humanized monoclonal antibodies generated from antibodies of the present invention. In certain embodiment, the antibody is OBI-888 (Anti-Globo H monoclonal antibody) Exemplary OBI-888 is as described in PCT patent publications (WO2015157629A2 and WO2017062792A1), patent applications, the contents of which are incorporated by reference in its entirety.

The antibodies can be full-length or can comprise a fragment (or fragments) of the antibody having an antigen-binding portion, including, but not limited to, Fab, F(ab′)₂, Fab′, F(ab)′, Fv, single chain Fv (scFv), bivalent scFv (bi-scFv), trivalent scFv (tri-scFv), Fd, dAb fragment (e.g., Ward et al, Nature, 341:544-546 (1989)), an CDR, diabodies, triabodies, tetrabodies, linear antibodies, single-chain antibody molecules, and multispecific antibodies formed from antibody fragments. Single chain antibodies produced by joining antibody fragments using recombinant methods, or a synthetic linker, are also encompassed by the present invention. Bird et al. Science, 1988, 242:423-426. Huston et al, Proc. Natl. Acad. Sci. USA, 1988, 85:5879-5883.

The antibodies or antigen-binding portions thereof of the present invention may be monospecific, bi-specific or multispecific.

All antibody isotypes are encompassed by the present invention, including IgG (e.g., IgG₁, IgG₂, IgG₃, IgG₄), IgM, IgA (IgA₁, IgA₂), IgD or IgE (all classes and subclasses are encompassed by the present invention). The antibodies or antigen-binding portions thereof may be mammalian (e.g., mouse, human) antibodies or antigen-binding portions thereof. The light chains of the antibody may be of kappa or lambda type.

Thus, anti-cancer antibodies of the present invention include in combination with a heavy chain or light chain variable region, a heavy chain or light chain constant region, a framework region, or any portion thereof, of non-murine origin, preferably of human origin, which can be incorporated into an antibody of the present invention.

Antibodies with a variable heavy chain region and a variable light chain region that are at least about 70%, at least about 75%, at least about 80%, at least about 81%, at least about 82%, at least about 83%, at least about 84%, at least about 85%, at least about 86% o, at least about 87%>, at least about 88%>, at least about 89%>, at least about 90%>, at least about 91>, at least about 92%>, at least about 93%>, at least about 94%>, at least about 95%), at least about 96%>, at least about 97%>, at least about 98%>, at least about 99%> or about 100% homologous to the variable heavy chain region and variable light chain region of the antibody produced by the reference antibody, and can also bind to Globo series antigens (Globo-H, SSEA-3 and SSEA-4). Homology can be present at either the amino acid or nucleotide sequence level.

The antibodies or antigen-binding portions may be peptides. Such peptides can include variants, analogs, orthologs, homologs and derivatives of peptides, that exhibit a biological activity, e.g., binding of a carbohydrate antigen. The peptides may contain one or more analogs of an amino acid (including, for example, non-naturally occurring amino acids, amino acids which only occur naturally in an unrelated biological system, modified amino acids from mammalian systems etc.), peptides with substituted linkages, as well as other modifications known in the art.

Also within the scope of the invention are antibodies or antigen-binding portions thereof in which specific amino acids have been substituted, deleted or added. In an exemplary embodiment, these alternations do not have a substantial effect on the peptide's biological properties such as binding affinity. In another exemplary embodiment, antibodies may have amino acid substitutions in the framework region, such as to improve binding affinity of the antibody to the antigen. In yet another exemplary embodiment, a selected, small number of acceptor framework residues can be replaced by the corresponding donor amino acids. The donor framework can be a mature or germline human antibody framework sequence or a consensus sequence. Guidance concerning how to make phenotypically silent amino acid substitutions is provided in Bowie et al., Science, 247:1306-1310 (1990). Cunningham et al, Science, 244:1081-1085 (1989). Ausubel (ed.), Current Protocols in Molecular Biology, John Wiley and Sons, Inc. (1994). T. Maniatis, E. F. Fritsch and J. Sambrook, Molecular Cloning: A Laboratory Manual, Cold Spring Harbor laboratory, Cold Spring Harbor, N.Y. (1989). Pearson, Methods Mol. Biol. 243:307-31 (1994). Gonnet et al., Science 256:1443-45 (1992).

The antibody, or antigen-binding portion thereof, can be derivatized or linked to another functional molecule. For example, an antibody can be functionally linked (by chemical coupling, genetic fusion, noncovalent interaction, etc.) to one or more other molecular entities, such as another antibody, a detectable agent, a cytotoxic agent, a pharmaceutical agent, a protein or peptide that can mediate association with another molecule (such as a streptavidin core region or a polyhistidine tag), amino acid linkers, signal sequences, immunogenic carriers, or ligands useful in protein purification, such as glutathione-S-transferase, histidine tag, and staphylococcal protein A. One type of derivatized protein is produced by crosslinking two or more proteins (of the same type or of different types). Suitable crosslinkers include those that are heterobifunctional, having two distinct reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, 111. Useful detectable agents with which a protein can be derivatized (or labeled) include fluorescent compounds, various enzymes, prosthetic groups, luminescent materials, bioluminescent materials, and radioactive materials. Non-limiting, exemplary fluorescent detectable agents include fluorescein, fluorescein isothiocyanate, rhodamine, and, phycoerythrin. A protein or antibody can also be derivatized with detectable enzymes, such as alkaline phosphatase, horseradish peroxidase, beta-galactosidase, acetylcholinesterase, glucose oxidase and the like. A protein can also be derivatized with a prosthetic group (e.g., streptavidin/biotin and avidin/biotin).

Nucleic acids encoding a functionally active variant of the present antibody or antigen-binding portion thereof are also encompassed by the present invention. These nucleic acid molecules may hybridize with a nucleic acid encoding any of the present antibody or antigen-binding portion thereof under medium stringency, high stringency, or very high stringency conditions. Guidance for performing hybridization reactions can be found in Current Protocols in Molecular Biology, John Wiley & Sons, N.Y. 6.3.1-6.3.6, 1989, which is incorporated herein by reference. Specific hybridization conditions referred to herein are as follows: 1) medium stringency hybridization conditions: 6×SSC at about 45° C., followed by one or more washes in 0.2×SSC, 0.1% SDS at 60° C.; 2) high stringency hybridization conditions: 6×SSC at about 45° C., followed by one or more washes in 0.2×SSC, 0.1% SDS at 65° C.; and 3) very high stringency hybridization conditions: 0.5 M sodium phosphate, 7% SDS at 65° C., followed by one or more washes at 0.2×SSC, 1% SDS at 65° C.

A nucleic acid encoding the present antibody or antigen-binding portion thereof may be introduced into an expression vector that can be expressed in a suitable expression system, followed by isolation or purification of the expressed antibody or antigen-binding portion thereof. Optionally, a nucleic acid encoding the present antibody or antigen-binding portion thereof can be translated in a cell-free translation system. U.S. Pat. No. 4,816,567. Queen et al, Proc Natl Acad Sci USA, 86:10029-10033 (1989).

The present antibodies or antigen-binding portions thereof can be produced by host cells transformed with DNA encoding light and heavy chains (or portions thereof) of a desired antibody. Antibodies can be isolated and purified from these culture supernatants and/or cells using standard techniques. For example, a host cell may be transformed with DNA encoding the light chain, the heavy chain, or both, of an antibody. Recombinant DNA technology may also be used to remove some or all of the DNA encoding either or both of the light and heavy chains that is not necessary for binding, e.g., the constant region.

The present nucleic acids can be expressed in various suitable cells, including prokaryotic and eukaryotic cells, e.g., bacterial cells, (e.g., E. coli), yeast cells, plant cells, insect cells, and mammalian cells. A number of mammalian cell lines are known in the art and include immortalized cell lines available from the American Type Culture Collection (ATCC). Non-limiting examples of the cells include all cell lines of mammalian origin or mammalian-like characteristics, including but not limited to, parental cells, derivatives and/or engineered variants of monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells. The engineered variants include, e.g., glycan profile modified and/or site-specific integration site derivatives.

The present invention also provides for cells comprising the nucleic acids described herein. The cells may be a hybridoma or transfectant.

Alternatively, the present antibody or antigen-binding portion thereof can be synthesized by solid phase procedures well known in the art. Solid Phase Peptide Synthesis: A Practical Approach by E. Atherton and R. C. Sheppard, published by IRL at Oxford University Press (1989). Methods in Molecular Biology, Vol. 35: Peptide Synthesis Protocols (ed. M. W. Pennington and B. M. Dunn), chapter 7. Solid Phase Peptide Synthesis, 2nd Ed., Pierce Chemical Co., Rockford, IL (1984). G. Barany and R. B. Merrifield, The Peptides: Analysis, Synthesis, Biology, editors E. Gross and J. Meienhofer, Vol. 1 and Vol. 2, Academic Press, New York, (1980), pp. 3-254. M. Bodansky, Principles of Peptide Synthesis, Springer-Verlag, Berlin (1984).

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

The term “hypervariable region”, “HVR”, or “HV”, when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)).

“Framework” or “FW” residues are those variable domain residues other than the hypervariable region residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO93/1161; and Hollinger et al., Proc. Natl. Acad. Sci. USA 90:6444-6448 (1993).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more HVRs thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154 (7): 3310-9 (1995); and Hawkins et al, J. Mol. Biol. 226:889-896 (1992).

A “blocking” antibody or an “antagonist” antibody is one which inhibits or reduces biological activity of the antigen it binds. Certain blocking antibodies or antagonist antibodies substantially or completely inhibit the biological activity of the antigen.

An “agonist antibody”, as used herein, is an antibody which mimics at least one of the functional activities of a polypeptide of interest.

A “disorder” is any condition that would benefit from treatment with an antibody of the invention. This includes chronic and acute disorders or diseases including those pathological conditions which predispose the mammal to the disorder in question. Non-limiting examples of disorders to be treated herein include cancer.

The terms “cell proliferative disorder” and “proliferative disorder” refer to disorders that are associated with some degree of abnormal cell proliferation. In one embodiment, the cell proliferative disorder is cancer.

“Tumor” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing or decreasing inflammation and/or tissue/organ damage, decreasing the rate of disease progression, amelioration or palliation of the disease state, and remission or improved prognosis. In some embodiments, antibodies of the invention are used to delay development of a disease or disorder.

An “individual” or a “subject” is a vertebrate. In certain embodiments, the vertebrate is a mammal. Mammals include, but are not limited to, farm animals (such as cows), sport animals, pets (such as cats, dogs, and horses), primates, mice and rats. In certain embodiments, the vertebrate is a human.

“Mammal” for purposes of treatment refers to any animal classified as a mammal, including humans, domestic and farm animals, and zoo, sports, or pet animals, such as dogs, horses, cats, cows, etc. In certain embodiments, the mammal is human.

An “effective amount” refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount is also one in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount would be less than the therapeutically effective amount.

One aspect of the present disclosure features anti-Globo H monoclonal antibody. The Anti-Globo H antibody binds to Fucα1→2 Galβ1→3 GalNAcβ1→3 Galα1→4 Galβ1→4 Glc.

Any of the antibodies described herein can be a full length antibody or an antigen-binding fragment thereof. In some examples, the antigen binding fragment is a Fab fragment, a F(ab′)₂ fragment, or a single-chain Fv fragment. In some examples, the antigen binding fragment is a Fab fragment, a F(ab′)₂ fragment, or a single-chain Fv fragment. In some examples, the antibody is a human antibody, a humanized antibody, a chimeric antibody, or a single-chain antibody.

Any of the antibodies described herein has one or more characteristics of: (a) is a recombinant antibody, a monoclonal antibody, a chimeric antibody, a humanized antibody, a human antibody, an antibody fragment, a bispecific antibody, a monospecific antibody, a monovalent antibody, an IgG₁ antibody, an IgG₂ antibody, or derivative of an antibody; (b) is a human, murine, humanized, or chimeric antibody, antigen-binding fragment, or derivative of an antibody; (c) is a single-chain antibody fragment, a multibody, a Fab fragment, and/or an immunoglobulin of the IgG, IgM, IgA, IgE, IgD isotypes and/or subclasses thereof; (d) has one or more of the following characteristics: (i) mediates ADCC and/or CDC of cancer cells; (ii) induces and/or promotes apoptosis of cancer cells; (iii) inhibits proliferation of target cells of cancer cells; (iv) induces and/or promotes phagocytosis of cancer cells; and/or (v) induces and/or promotes the release of cytotoxic agents; (c) specifically binds the tumor-associated carbohydrate antigen, which is a tumor-specific carbohydrate antigen; (f) does not bind an antigen expressed on non-cancer cells, non-tumor cells, benign cancer cells and/or benign tumor cells; and/or (g) specifically binds a tumor-associated carbohydrate antigen expressed on cancer stem cells and on normal cancer cells.

Preferably the binding of the antibodies to their respective antigens is specific. The term “specific” is generally used to refer to the situation in which one member of a binding pair will not show any significant binding to molecules other than its specific binding partner(s) and e.g. has less than about 30%, preferably 20%, 10%, or 1% cross-reactivity with any other molecule other than those specified herein.

### Humanized Antibodies

The invention encompasses humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies can be important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody (Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623.

It is further generally desirable that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

In some embodiments, the methods as disclosed herein are useful for the treatment or prevention of a cancer, for example where a cancer is characterized by increased Globo-H, SSEA-3 and/or SSEA-4 expression. In some embodiments the cancer comprises a cancer stem cell. In some embodiments, the cancer is a pre-cancer, and/or a malignant cancer and/or a therapy resistant cancer. In some embodiments, the cancer is a brain cancer.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having cancer, which include, but not limited to, sarcoma, skin cancer, leukemia, lymphoma, brain cancer, lung cancer, breast cancer, oral cancer, esophagus cancer, gastric cancer, liver cancer, bile duct cancer, pancreas cancer, colon cancer, kidney cancer, cervix cancer, ovary cancer and prostate cancer. A subject having cancer can be identified by routine medical examination.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

As used herein, the term “treating” refers to the application or administration of a composition including one or more active agents to a subject, who has cancer, a symptom of cancer, or a predisposition toward cancer, with the purpose to cure, heal, alleviate, relieve, alter, remedy, ameliorate, improve, or affect cancer, the symptom of cancer, or the predisposition toward cancer.

“Development” or “progression” of cancer means initial manifestations and/or ensuing progression of cancer. Development of cancer can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of cancer includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble formulations can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Validation of Globo-H IHC for Seven Cancer Types Application

### 1. Purpose

The purpose of this study was to validate an immunohistochemistry (IHC) assay for the detection of Globo-H expression in human pancreatic, lung, gastric, colorectal, liver, and esophageal cancer specimens. A Globo-H assay validated in breast cancer will be used to confirm Globo-H IHC staining in the above indications. NeoGenomics validated the Globo-H IHC assay in human pancreatic, lung, gastric, colorectal, liver, and esophageal cancer by assessing accuracy, sensitivity, specificity and precision. Indication specific tumor TMAs were also screened using the same Globo-H IHC assay.

### 2. Definitions & Acronyms

| Word/Acronym | Definition |
|---|---|
| μm | Micrometer or micron |
| CRC | Colorectal cancer |
| CV | Coefficient of variance |
| FDA | Food and Drug Administration |
| FFPE | Formalin-fixed paraffin-embedded |
| Globo-H | Glycosphingolipid of the globo series |
| | with a sugar terminus resembling |
| | the blood group antigen H determinant |
| HCC | Hepatocellular carcinoma |
| IHC | Immunohistochemistry |
| N/A or NA | Not applicable |
| NSCLC | Non-small cell lung cancer |
| TMA | Tissue micro-array |

### 3. Associated Reagents/Probes/Antibodies

| | Lot | | Storage |
|---|---|---|---|
| Name | Number | Expiration | Temperature |
| Anti-Globo-H antibody, VK9, eBioscience | 4306278 | MAY 2021 | 2-8° C. |
| (ThermoFisher, Cat# 14-9700-82) | | | |
| MCF-7; 105-EN-024 Grl-5/Grl-7 xenograft FFPE block | N/A | N/A | Ambient |
| MDA-MB-231: 105-EN-065 #1-4 xenograft FFPE | N/A | N/A | Ambient |
| block | | | |
| HPAC: 105-EN-051 Grl-7 xenograft FFPE block | N/A | N/A | Ambient |
| EnVision ™ FLEX, High pH, (Link) Kit (Dako, Cat# | 10132556 | OCTOBER 2018 | 2-8° C. |
| K8002): | 10130945 | AUGUST 2018 | |
| Envision FLEX Target Retrieval Solution, High pH | 10129395 | JULY 2018 | |
| EnVision FLEX Peroxidase-Blocking Reagent | | | |
| EnVision FLEX/HRP | | | |
| Envision FLEX Wash Buffer 20X | | | |
| DAB Substrate Buffer & Chromogen DAB | | | |
| DAB Substrate Buffer & Chromogen | | | |
| EnVision ™ FLEX Target Retrieval Solution, Low pH | 10133261 | | 2-8° C. |
| (Dako, Cat# K8005) | 10129373 | | |
| | 10121667 | | |
| | 10128667 | JULY 2018 | |
| | 10123663 | MAY 2018 | |
| | 10125515 | | |
| Antibody Diluent with Background-Reducing | 10131857 | MAY 2019 | 2-8° C. |
| Components (Dako, Cat# S3022) | 10129260 | JANUARY 2019 | |
| EnVision ™ FLEX Hematoxylin (Dako, Cat# K8008) | 10132837 | NOVEMBER 2018 | Ambient |
| | 10130055 | SEPTEMBER 2018 | |
| Purified Mouse IgG3 Isotype Control Antibody | B220934 | DECEMBER 2018 | 2-8° C. |
| (BioLegend, Cat# 401302) | | | |
| Deionized Water | N/A | N/A | Ambient |
| 100% Alcohol | N/A | N/A | Ambient |
| 95% Alcohol | N/A | N/A | Ambient |
| Xylene | N/A | N/A | Ambient |

### 4. Associated Equipment

| Name | Model |
|---|---|
| Microtome | Leica RM2235 or equivalent |
| Flotation Bath | TBS TFBL or equivalent |
| Drying oven | Biocare 10-180Aer or equivalent |
| Antigen Retrieval Chamber | Dako PT Link |
| Slide stainer | Dako Link 48 |
| Linear stainer | Leica Autostainer XL or equivalent |
| Cover slipper | Tissue-Tek Film 4740 or equivalent |

### 5. Specimen Requirements

### 5.1 Storage Time and Temperature:

FFPE tissue blocks were sectioned at 4-5 μm and mounted on positively charged slides. Slides were air-dried and stored throughout the duration of the study at room temperature.

### 5.2 Unacceptable Specimens:

Specimens that are not FFPE.

### 6. Specimen De-Identification

### 6.1 Source of Specimen:

Tumor specimens and tumor indication specific TMA slides were provided by NeoGenomics as FFPE tissue blocks or slides from a qualified vendor and the AGI tissue bank. Cell line control blocks were provided by the Sponsor.

### 6.2 Description of De-Identification Process:

Specimens were labeled with identifiers that NeoGenomics cannot trace back to the patient.

### Study Design

### 7. Globo-H Assay Controls and IHC Scoring

7.1 Batch control tissues used in each staining run consisted of a known positive cell line block (HPAC), confirmed by LC-MS/MS, and a human breast cancer tissue (from AGI tissue bank) containing elements with positive and negative Globo-H expression; these controls were all stained with Globo-H antibody and negative control antibody. Representative images of the human breast cancer tissue control are shown in FIGS. 1A to 1F. FIGS. 1A to 1F show the Globo-H IHC staining of the breast cancer control tissue. FIG. 1A shows the Globo-H staining in tumor region, FIG. 1B show in high magnification the Globo-H staining in tumor region, FIG. 1C shows the Globo-H staining in non-tumor region, FIG. 1D shows in high magnification the Globo-H staining in non-tumor region, FIG. 1E shows the Negative control antibody staining in tumor region, and FIG. 1F shows the Negative control antibody staining in normal breast region.

7.2 Semi-quantitative Globo-H pathologist evaluation criteria were established during the breast cancer validation and unchanged for this validation.

7.3 Globo-H IHC stained slides were evaluated by a Neogenomics pathologist using a brightfield microscope.

7.4 Globo-H IHC expression was assessed in tumor cells. The percent of tumor cells with membrane and/or cytoplasmic (in a granular or diffuse pattern) Globo-H expression was recorded at each staining intensity (0, 1+, 2+, and The intensity of the Globo H expression was reported as 0 for no detectable staining; 1+ for translucent or low level staining; 2+ for moderate or opaque staining; and 3+ for strong or solid staining. The H-score values range from 0 to 300 and is calculated with the following formula:

$$H - score = [ ( \% of Tumor Cell Membrane / Cytoplasmic at 1+ ) \times 1 + ( \% of Tumor Cell Membrane / Cytoplasmic at 2+ ) \times 2 + ( \% of Tumor Cell Membrane / Cytoplasmic at 3+ ) \times 3 ]$$

FIGS. 2A to 2E show the Globo-H IHC staining intensities in a NSCLC specimen. FIG. 2A shows the Heterogeneous Globo-H staining of NSCLC cells. FIG. 2B shows intense staining, FIG. 2C shows moderate staining, FIG. 2D shows weak staining and FIG. 2E shows no staining.

7.5 The pathologist also recorded if nuclear expression was observed.

| Specimen | Negative | | Nuclear | Comments |
|---|---|---|---|---|
| ID | Control | Tumor Cell Membrane/Cytoplasmic | Staining | |
| | | Percent Positive (%) | Observed? | |

| | | | | |
|---|---|---|---|---|
| 0 | 1+ | 2+ | 3+ | Y/N |

7.6 A semi-quantitative H-score assessment of Globo-H positive cells was calculated and reported.

THE standard H-score methodology was used as an approach to measuring Globo-H expression in a semi-quantitative fashion. Based on the cell membrane and cytosolic translocating feature of Globo H, the cells with positive signal on the cell membrane or cytosolic are considered positive staining. This scoring methodology allows the assessment of Globo H immunoexpression that takes into account both the percentage of cells expressing this antigen and the intensity (level of expression) per cell.

The score is obtained by reviewing specimens based on a 0-3+ intensity gradient and determining the percentage of cells expressing the marker at each of the 4 levels of intensity (0, 1+ (weakly staining), 2+ (moderately staining), 3+ (strongly staining) using the formula: H score=[(% of Tumor Cell Membrane and/or Cytoplasmic at 1+)×1+(% of Tumor Cell Membrane and/or Cytoplasmic at 2+)×2+(% of Tumor Cell Membrane and/or Cytoplasmic at 3+)×3], yielding a dynamic range of 0-300.

For GLOBO-H, the specimen is assessed for the percentage of Globo-H IHC staining of tumor cells (primarily cytoplasmic staining) across the 0-3+ intensity levels, and the H-score is calculated accordingly, as described above.

7.7 The sponsor assessed H-score cutoffs of ≥1, ≥15, ≥20, ≥100, and ≥150. Prevalence and concordance is reported using the above cutoff values.

### 8. Globo-H IHC Confirmation

### 8.1. Study Setup

The Globo-H IHC assay, as performed in the breast cancer validation, was used to stain specimens from each of 6 tumor indications (pancreatic, lung, gastric, colorectal, liver, and esophageal cancer). Minor adjustments to the assay protocol may have been incorporated to optimize specific staining and reduce background staining under the guidance of a qualified NeoGenomics pathologist. Globo-H IHC pathologist evaluation criteria developed for breast cancer was reviewed and confirmed for each of the 6 tumor indications. Some adjustments to the evaluation criteria may have been established, per indication, with guidance from a NeoGenomics pathologist and OBI review and approval. A cut-off, per indication was later communicated by OBI.

### 8.2. Results

Based on confirmation staining from each of 6 tumor indications, no adjustments to the assay protocol were required. Pathologist evaluation criteria was updated to specify that granular staining within the cytoplasm is acceptable and should be scored based on staining observed in pancreatic tumor cells as well as other indications. The validated Globo-H assay using the Globo-H mouse monoclonal antibody (VK9) on the Dako Link 48 platform was confirmed. The Globo-H IHC assay is summarized below.

Briefly, tissues were sectioned at 4-5 μm, mounted on positively charged slides, and allowed to air-dry. Slides were then heated in a 58° C. drying oven for 60 minutes. Deparaffinization and epitope retrieval was performed using EnVision FLEX Low PH TRS (Agilent) in the Dako PT Link module for 20 minutes at 97° C. Slides were allowed to cool to 65° C., then removed from the PT Link and placed in a FLEX wash buffer bath for 5 minutes. Reagents from the En Vision™ FLEX, High pH Kit were used to perform the following steps on the Dako Link 48 IHC platform. In between each step, slides were rinsed with deionized water or FLEX wash buffer, as noted in Table 1. Slides were treated with En Vision FLEX Peroxidase Block for 5 minutes followed by Globo-H primary antibody (10 μg/mL, 1:50 in Dako Background Reducing Diluent) incubation for 60 minutes. Visualization was achieved with En Vision FLEX HRP for 30 minutes and EnVision FLEX DAB+ for 10 minutes. Slides were counterstained on the Dako Link 48 using EnVision FLEX Hematoxylin for 5 minutes, then removed from the instrument. Slides were dehydrated using a series of graded alcohol and cleared with xylene prior to being coverslipped using a Tissue-Tek automated film coverslipper.

Images of the confirmed protocol in pan-cancer tissues are displayed in FIGS. 3A to 3F. The processing steps of the Globo-H IHC assay are listed in Table 1 and diagramed in FIG. 4 Error! Reference source not found. A schematic image of the detection system binding is displayed in FIG. 5 Error! Reference source not found. The Globo-H IHC assay was validated in pan-cancer, assessing accuracy, sensitivity, specificity, and intra-run and inter-run reproducibility in the following sections.

TABLE 1
Globo-H IHC Protocol on the Dako LINK 48 - Clone VK9

| Reagent | Incubation Time | | Dispense Volume | Temperature | |
|---|---|---|---|---|---|
| Tissue Sectioning (4-5 μm) | N/A | | N/A | Ambient | |
| Labeling and Storage | N/A | | N/A | Ambient | |
| Bake Slides | 60 | min | N/A | 58-62° | C. |
| Print Dako Labels | N/A | | N/A | Ambient | |
| PT Link Antigen Retrieval - | 20 | min | N/A | 97° | C. |

| Low pH TRS | | | | |
|---|---|---|---|---|
| Wash Buffer | 5 | min | N/A | Ambient |
| Wash Buffer Rinse | 0 | min | N/A | Ambient |
| Peroxide Block | 5 | min | 300 μl | Ambient |
| | | | (150 μl × 2 zones) | |
| Wash Buffer Rinse | 0 | min | N/A | Ambient |
| Globo-H Antibody | 60 | min | 300 μl | Ambient |
| (10 μg/mL, 1:50) | | | (150 μl × 2 zones) | |
| Wash Buffer Rinse | 0 | min | N/A | Ambient |
| FLEX HRP | 30 | min | 300 μl | Ambient |
| | | | (150 μl × 2 zones) | |
| Wash Buffer Rinse | 0 | min | N/A | Ambient |
| Wash Buffer | 5 | min | N/A | Ambient |
| FLEX DAB+ | 10 | min | 300 μl | Ambient |
| | | | (150 μl × 2 zones) | |
| Wash Buffer Rinse | 0 | min | N/A | Ambient |
| FLEX Hematoxylin | 5 | min | 300 μl | Ambient |
| | | | (150 μl × 2 zones) | |
| Deionized Water | 0 | sec | N/A | Ambient |
| Wash Buffer Rinse | 5 | min | N/A | Ambient |
| Deionized Water | 0 | sec | N/A | Ambient |
| Unload from Dako, | 7 | minutes | N/A | ambient |

| | | | |
|---|---|---|---|
| Dehydrate, and Clear | | | |
| Coverslip | NA | N/A | ambient |

### 9. Accuracy

### 9.1. Study Setup

HPAC and SK-BR3 cell line blocks, provided by the Sponsor, were stained and evaluated by NeoGenomics using the approved Globo-H IHC and pathologist evaluation procedures. Results were compared to Sponsor data by testing the same cell lines by mass spectrometry.

### 9.2. Results

RESULTS from staining HPAC and SK-BR3 cell line blocks with the Globo-H IHC assay are listed in Table 2. Representative images of the cell line blocks are displayed in FIG. 6 Error! Reference source not found. A (showing Globo-H IHC in HPAC Cell Line) and FIG. 6B (showing Globo-H IHC in SK-BR3 Cell Line). Globo-H expression was observed in the HPAC cell line, but not in the SK-BR3 cell line.

TABLE 2
Globo-H IHC Expression in - Cell Line Controls

| | | Tumor Cell | | | | | | Nuclear |
|---|---|---|---|---|---|---|---|---|
| | | Membrane/Cytoplasmic Percent | | | | | | Staining |
| Specimen | Negative | Positive (%) | | | | Percent | | Observed? |
| ID | Control | 0 | 1+ | 2+ | 3+ | Positive | H-Score | Y/N |
| OBI-A | 0 | 95 | 0 | 3 | 2 | 5 | 12 | N |
| (HPAC) | | | | | | | | |
| OBI-E | 0 | 100 | 0 | 0 | 0 | 0 | 0 | N |
| (SK-BR3) | | | | | | | | |

### 9.3. Acceptability Criteria

ACCURACY of tested specimens compared to Sponsor data is ≥85% or approved by the Sponsor.

### 9.4. Conclusion

Results from IHC staining of HPAC (positive) and SK-BR3 (negative) cell lines was consistent with Sponsor data from mass spectrometry.

### 10. Sensitivity

### 10.1. Study Setup

ONE hundred fifty-four (154) tumor specimens (29 CRC, 24 esophageal, 25 gastric, 23 HCC, 29 NSCLC, and 24 pancreatic) and a tumor TMA for each indication were stained and evaluated using the confirmed Globo-H IHC and pathologist evaluation procedures. A negative control antibody was included for each specimen when slides were available. An H&E stain was performed on each specimen to aid in pathologist evaluation (for morphological/histological reference). Batch control tissues used in each staining run consisted of a known positive cell line block (HPAC), confirmed by LC-MS/MS, and a human breast cancer tissue (from AGI tissue bank) containing elements with positive and negative Globo-H expression; these controls were all stained with Globo-H antibody and negative control antibody. Due to limited data on Globo-H expression in the tested indications, the results of tumor specimens were reported as found.

### 10.2. Results

RESULTS from staining 154 tumor specimens are listed in Appendix 1. Results from staining 6 tumor indication specific TMAs are listed in Appendix 2. The distribution of Globo-H IHC H-scores per indication are shown in FIG. 7 Error! Reference source not found. Globo-H prevalence for each tumor indication was assessed at 5 cutoffs, H-score ≥1, ≥15, ≥20, ≥100, and ≥150, for resections (Table 3 Error! Reference source not found, as shown in FIG. 25), TMAs (Table 4 Error! Reference source not found, as shown in FIG. 26), and overall (Table 5, as shown in FIG. 27). HCC specimens had the lowest prevalence (7.1%, 5.7%, 0.0%, and 0.0%), while pancreatic cancer had the highest prevalence (66.7%, 66.7%, 50.0%, and 40.3%) using cutoffs of H-score ≥15, ≥20, ≥100, and ≥150, respectively.

Schematic images of tumor resection specimens identifying Globo-H IHC staining in tumor cells and non-tumor areas are displayed in FIGS. 8-13. Representative images of TMAs stained with the Globo-H IHC assay are displayed in FIG. 14.

FIG. 8 shows Schematic Representative Image of Globo-H IHC in a CRC Resection Specimen; CRC Specimen (00-14571-A11) with low and high magnification images of Globo-H IHC staining in tumor and non-tumor sites, tumor H-score=190. Blue head indicates Globo H positive immune cells.

FIG. 9 shows Schematic Representative Image of Globo-H IHC in an Esophageal Cancer Resection Specimen; Esophageal cancer specimen (F00044229) with low and high magnification images of Globo-H IHC staining in tumor and non-tumor sites, tumor H-score=60. Blue head indicates Globo H positive immune cells.

FIG. 10 shows Schematic Representative Image of Globo-H IHC in a Gastric Cancer Resection Specimen; Gastric cancer specimen (F00042004) with low and high magnification images of Globo-H IHC staining in tumor and non-tumor sites, tumor H-score=105. Blue head indicates Globo H positive immune cells.

FIG. 11 shows Schematic Representative Image of Globo-H IHC in a HCC Resection SPECIMEN; HCC specimen (F00083288) with low and high magnification images of Globo-H IHC staining in tumor and non-tumor sites, tumor H-score=15.

FIG. 12 shows Schematic Representative Image of Globo-H IHC in a NSCLC Resection Specimen; NSCLC specimen (99-11977-A2) with low and high magnification images of Globo-H IHC staining in tumor and non-tumor sites, tumor H-score=130. Blue head indicates Globo H positive IMMUNE cells.

FIG. 13 shows Schematic Representative Image of Globo-H IHC in a Pancreatic Cancer Resection SPECIMEN; Pancreatic cancer specimen (93-8057-40) with low and high magnification images of Globo-H IHC staining in tumor and non-tumor sites, tumor H-score=290. Blue head indicates Globo H positive immune cells.

FIGS. 14A to 14L show Globo-H IHC in Whole Specimens and TMA cores from 6 Tumor Indications.

### 10.3. Acceptability Criteria

SENSITIVITY of tested specimens compared to Sponsor data (if available) is ≥85% or approved by the Sponsor. If no Sponsor data is available, results will be reported as found and must be approved by the Sponsor.

### 10.4. Conclusion

THE prevalence of Globo-H IHC in the tested indications ranged from 7.1%-66.7%, 5.7%-66.7%, 0.0%-50.0%, and 0.0%-40.3% using cutoffs of H-score ≥15 (cut-off used for breast cancer, CT Validation 230)⁶, ≥20, ≥100, and ≥150; the data was approved by the Sponsor.

### 11. Specificity

### 11.1. Study Setup

Data from the Sensitivity study were used to determine the negative prevalence (percentage of specimens that are negative for Globo-H) of Globo-H IHC staining in the tested indications. Additionally, an FDA normal TMA was previously stained with the approved Globo-H IHC assay during the breast cancer validation. The data are reported as found.

### 11.2. Results

GLOBO-H negative prevalence for each tumor indication was assessed at 3 cutoffs, H-score ≥1, ≥15, and ≥20, for resections (Table 5-1, as shown in FIG. 28), TMAs (Table 6, as shown in FIG. 29), and overall (Table 7, as shown in FIG. 30). Pancreatic cancer specimens had the lowest negative prevalence (33.3% and 33.3%), while HCC had the highest negative prevalence (92.9% and 94.3%) using cutoffs of H-score ≥15 and ≥20, respectively. Results from the FDA normal tissue TMA are listed in Appendix 3. A summary of Globo-H IHC expression by normal tissue type is summarized in Table 8 Error! Reference source not found. (as shown in FIG. 31).

### 11.3. Acceptability Criteria

SPECIFICITY of tested specimens compared to Sponsor data (if available) is ≥85% or approved by the Sponsor. If no Sponsor data is available, results will be reported as found and must be approved by the Sponsor.

### 11.4 Conclusion

The negative PREVALENCE of Globo-H IHC in the tested indications ranged from 33.3%-92.9%, 33.3%-94.3%, 50.0%-100.0%, and 59.7%-100.0% using cutoffs of H-score ≥15, ≥20, ≥100, and ≥150, respectively; the data was approved by the Sponsor.

### 12. Precision—Repeatability

### 12.1. Study Setup

Three (3) SPECIMENS per indication were selected by NeoGenomics in conjunction with the Sponsor for this study. The specimens were selected to represent the overall dynamic range of Globo-H IHC expression, with emphasis on the cut-off. Five sections from each specimen were cut and stained on one run, using the approved Globo-H IHC assay. An additional slide per specimen was stained using an isotype control antibody. Slides for each specimens were stained on the same day using the same operator, equipment, and reagents. Batch control tissues for repeatability consisted of a known positive cell line block (HPAC), confirmed by LC-MS/MS, and a human breast cancer tissue (from AGI tissue bank) containing elements with positive and negative Globo-H expression; these controls were stained with Globo-H antibody and negative control antibody. Stained slides were evaluated by a NeoGenomics pathologist using the scoring criteria established in Section 8. Concordance was determined based on 3 cutoffs provided by the Sponsor.

### 12.2. Results

Globo-H IHC repeatability testing results are shown in Table 9. Concordance around 4 cutoff values (H-score=1, 15, 20, 100, and 150) was determined and are listed in Table 10.

TABLE 9
Globo-H IHC Staining Results from the Repeatability Study

| Specimen | Staining | Isotype | | | | | | Total % | |
|---|---|---|---|---|---|---|---|---|---|
| ID | Date | Control | Slide | 0 | 1+ | 2+ | 3+ | Positive | H-Score |
| F00060972 | 20 FEB. 2018 | 0 | 1 | 0 | 0 | 0 | 100 | 100 | 300 |
| Esophageal | | | 2 | 0 | 0 | 0 | 100 | 100 | 300 |
| | | | 3 | 0 | 0 | 0 | 100 | 100 | 300 |
| | | | 4 | 0 | 0 | 0 | 100 | 100 | 300 |
| | | | 5 | 0 | 0 | 0 | 100 | 100 | 300 |
| F00060983 | 21 FEB. 2018 | 0 | 1 | 60 | 20 | 10 | 10 | 40 | 70 |
| Esophageal | | | 2 | 70 | 10 | 10 | 10 | 30 | 60 |
| | | | 3 | 70 | 10 | 10 | 10 | 30 | 60 |
| | | | 4 | 70 | 10 | 10 | 10 | 30 | 60 |
| | | | 5 | 70 | 10 | 10 | 10 | 30 | 60 |
| F00061012 | 21 FEB. 2018 | 0 | 1 | 90 | 5 | 0 | 5 | 10 | 20 |
| Esophageal | | | 2 | 90 | 5 | 0 | 5 | 10 | 20 |
| | | | 3 | 90 | 5 | 0 | 5 | 10 | 20 |
| | | | 4 | 90 | 5 | 0 | 5 | 10 | 20 |
| | | | 5 | 90 | 5 | 0 | 5 | 10 | 20 |
| 00-14556-A5 | 21 FEB. 2018 | 0 | 1 | 97 | 1 | 1 | 1 | 3 | 6 |
| CRC | | | 2 | 97 | 1 | 1 | 1 | 3 | 6 |
| | | | 3 | 97 | 1 | 1 | 1 | 3 | 6 |
| | | | 4 | 97 | 1 | 1 | 1 | 3 | 6 |
| | | | 5 | 97 | 1 | 1 | 1 | 3 | 6 |
| 99-9021-A4 | 20 FEB. 2018 | 0 | 1 | 20 | 10 | 0 | 70 | 80 | 220 |
| CRC | | | 2 | 20 | 10 | 0 | 70 | 80 | 220 |
| | | | 3 | 20 | 10 | 0 | 70 | 80 | 220 |
| | | | 4 | 20 | 10 | 0 | 70 | 80 | 220 |
| | | | 5 | 25 | 5 | 0 | 70 | 75 | 215 |
| 00-8225-A4 | 19 FEB. 2018 | 0 | 1 | 100 | 0 | 0 | 0 | 0 | 0 |
| CRC | | | 2 | 100 | 0 | 0 | 0 | 0 | 0 |
| | | | 3 | 100 | 0 | 0 | 0 | 0 | 0 |
| | | | 4 | 100 | 0 | 0 | 0 | 0 | 0 |
| | | | 5 | 100 | 0 | 0 | 0 | 0 | 0 |
| 99-4637-E1 | 20 FEB. 2018 | 0 | 1 | 98 | 1 | 0 | 1 | 2 | 4 |
| NSCLC | | | 2 | 98 | 1 | 0 | 1 | 2 | 4 |
| | | | 3 | 98 | 1 | 0 | 1 | 2 | 4 |
| | | | 4 | 98 | 1 | 0 | 1 | 2 | 4 |
| | | | 5 | 98 | 1 | 0 | 1 | 2 | 4 |
| 99-11848-F6 | 20 FEB. 2018 | 0 | 1 | 95 | 2 | 0 | 3 | 5 | 11 |
| NSCLC | | | 2 | 95 | 2 | 0 | 3 | 5 | 11 |
| | | | 3 | 95 | 2 | 0 | 3 | 5 | 11 |
| | | | 4 | 95 | 2 | 0 | 3 | 5 | 11 |
| | | | 5 | 95 | 2 | 0 | 3 | 5 | 11 |
| 98-17523-B2 | 19 FEB. 2018 | 0 | 1 | 20 | 0 | 0 | 80 | 80 | 240 |
| NSCLC | | | 2 | 20 | 0 | 0 | 80 | 80 | 240 |
| | | | 3 | 20 | 0 | 0 | 80 | 80 | 240 |
| | | | 4 | 20 | 0 | 0 | 80 | 80 | 240 |
| | | | 5 | 20 | 0 | 0 | 80 | 80 | 240 |
| 92-4644-B12 | 19 FEB. 2018 | 0 | 1 | 60 | 20 | 10 | 10 | 40 | 70 |
| Gastric | | | 2 | 60 | 20 | 10 | 10 | 40 | 70 |
| | | | 3 | 60 | 20 | 10 | 10 | 40 | 70 |
| | | | 4 | 60 | 20 | 10 | 10 | 40 | 70 |
| | | | 5 | 60 | 20 | 10 | 10 | 40 | 70 |
| 93-2141-B6 | 20 FEB. 2018 | 0 | 1 | 85 | 5 | 5 | 5 | 15 | 30 |
| Gastric | | | 2 | 85 | 5 | 5 | 5 | 15 | 30 |
| | | | 3 | 85 | 5 | 5 | 5 | 15 | 30 |
| | | | 4 | 85 | 10 | 5 | 0 | 15 | 20 |
| | | | 5 | 85 | 10 | 5 | 0 | 15 | 20 |
| 94-2834-A6 | 20 FEB. 2018 | 0 | 1 | 70 | 5 | 5 | 20 | 30 | 75 |
| Gastric | | | 2 | 70 | 5 | 5 | 20 | 30 | 75 |
| | | | 3 | 70 | 5 | 5 | 20 | 30 | 75 |
| | | | 4 | 70 | 5 | 5 | 20 | 30 | 75 |
| | | | 5 | 70 | 5 | 5 | 20 | 30 | 75 |
| 93-8057-40 | 19 FEB. 2018 | 0 | 1 | 10 | 0 | 0 | 90 | 90 | 270 |
| PDA | | | 2 | 10 | 0 | 0 | 90 | 90 | 270 |
| | | | 3 | 10 | 0 | 0 | 90 | 90 | 270 |
| | | | 4 | 10 | 0 | 0 | 90 | 90 | 270 |
| | | | 5 | 10 | 0 | 0 | 90 | 90 | 270 |
| 90-4304-4A | 19 FEB. 2018 | 0 | 1 | 100 | 0 | 0 | 0 | 0 | 0 |
| PDA | | | 2 | 100 | 0 | 0 | 0 | 0 | 0 |
| | | | 3 | 100 | 0 | 0 | 0 | 0 | 0 |
| | | | 4 | 100 | 0 | 0 | 0 | 0 | 0 |
| | | | 5 | 100 | 0 | 0 | 0 | 0 | 0 |
| F00026463 | 20 FEB. 2018 | 0 | 1 | 90 | 5 | 2 | 3 | 10 | 18 |
| PDA | | | 2 | 90 | 5 | 2 | 3 | 10 | 18 |
| | | | 3 | 90 | 5 | 2 | 3 | 10 | 18 |
| | | | 4 | 90 | 5 | 2 | 3 | 10 | 18 |
| | | | 5 | 90 | 5 | 0 | 5 | 10 | 20 |
| 92-148-6 | 21 FEB. 2018 | 0 | 1 | 100 | 0 | 0 | 0 | 0 | 0 |
| HCC | | | 2 | 100 | 0 | 0 | 0 | 0 | 0 |
| | | | 3 | 100 | 0 | 0 | 0 | 0 | 0 |
| | | | 4 | 100 | 0 | 0 | 0 | 0 | 0 |
| | | | 5 | 100 | 0 | 0 | 0 | 0 | 0 |
| F00084252 | 19 FEB. 2018 | 0 | 1 | 80 | 10 | 5 | 5 | 20 | 35 |
| HCC | | | 2 | 90 | 5 | 5 | 0 | 10 | 15 |
| | | | 3 | 90 | 5 | 5 | 0 | 10 | 15 |
| | | | 4 | 90 | 5 | 5 | 0 | 10 | 15 |
| | | | 5 | 90 | 5 | 5 | 0 | 10 | 15 |
| F00083288 | 19 FEB. 2018 | 0 | 1 | 85 | 5 | 5 | 5 | 15 | 30 |
| HCC | | | 2 | 85 | 5 | 5 | 5 | 15 | 30 |
| | | | 3 | 85 | 5 | 5 | 5 | 15 | 30 |
| | | | 4 | 85 | 5 | 5 | 5 | 15 | 30 |
| | | | 5 | 85 | 5 | 5 | 5 | 15 | 30 |

TABLE 10
Globo-H IHC Repeatability Concordance

| | | Concordance | Concordance | Concordance | Concordance | Concordance |
|---|---|---|---|---|---|---|
| | | of H- | of H- | of H- | of H- | of H- |
| Specimen | Indication | score ≥1 | score ≥15 | score ≥20 | score ≥100 | score ≥150 |
| F00060972 | Esophageal | 100% | 100% | 100% | 100% | 100% |
| F00060983 | Esophageal | 100% | 100% | 100% | 100% | 100% |
| F00061012 | Esophageal | 100% | 100% | 100% | 100% | 100% |
| 00-14556-A5 | CRC | 100% | 100% | 100% | 100% | 100% |
| 99-9021-A4 | CRC | 100% | 100% | 100% | 100% | 100% |
| 00-8225-A4 | CRC | 100% | 100% | 100% | 100% | 100% |
| 99-4637-E1 | NSCLC | 100% | 100% | 100% | 100% | 100% |
| 99-11848-F6 | NSCLC | 100% | 100% | 100% | 100% | 100% |
| 98-17523-B2 | NSCLC | 100% | 100% | 100% | 100% | 100% |
| 92-4644-B12 | Gastric | 100% | 100% | 100% | 100% | 100% |
| 93-2141-B6 | Gastric | 100% | 100% | 100% | 100% | 100% |
| 94-2834-A6 | Gastric | 100% | 100% | 100% | 100% | 100% |
| 93-8057-4O | PDA | 100% | 100% | 100% | 100% | 100% |
| 90-4304-4A | PDA | 100% | 100% | 100% | 100% | 100% |
| F00026463 | PDA | 100% | 100% | 80% | 100% | 100% |
| 92-148-6 | HCC | 100% | 100% | 100% | 100% | 100% |
| F00084252 | HCC | 100% | 100% | 80% | 100% | 100% |
| F00083288 | HCC | 100% | 100% | 100% | 100% | 100% |

| | | | | | |
|---|---|---|---|---|---|
| Overall Concordance | 100% | 100% | 98% | 100% | 100% |

### 12.3. Acceptability Criteria

The average percent CV (% CV) of the results of the five slides from each specimen stained on one run must be ≤20% and/or the concordance ≥85% (if a cutoff is provided) or approved by the Sponsor.

### 12.4. Conclusion

The GLOBO-H IHC repeatability study resulted in overall concordance of 100%, 100%, 98%, 100%, and 100% using H-score cutoffs of ≥1, ≥15, ≥20, ≥100, and ≥150, respectively. These values met the acceptability criteria.

### 13. Precision—Reproducibility

### 13.1. Study Setup

Three (3) specimens per indication were selected by NeoGenomics in conjunction with the Sponsor for this study. The specimens were selected to represent the overall dynamic range of Globo-H IHC expression, with emphasis on the cut-off. These were the same specimens from repeatability. Five sections each specimen were stained on 5 independent staining runs, on non-consecutive days using the Globo-H IHC assay. An isotype matched IgG negative control was included for each specimen on each run. Batch control tissues used in each reproducibility staining run consisted of a known positive cell line block (HPAC), confirmed by LC-MS/MS (Appendix 4), and a human breast cancer tissue (from AGI tissue bank) containing elements with positive and negative Globo-H expression; these controls were all stained with Globo-H antibody and negative control antibody. Slides from repeatability were used as one of the inter-runs. Inter operator and inter-instrument comparisons were integrated into this procedure. The staining structure is listed below:
Run 1 was extracted from slide 5 of the Repeatability run above (operator 1, instrument 1)
Run 2: operator 1, instrument1
Run 3: operator 1, instrument1.
Run 4: operator 1, instrument 2.
Run 5: operator 2, instrument 1

Stained slides were evaluated by a NeoGenomics pathologist using the scoring criteria in Section 8. Inter-run (Runs 1, 2, 3, 4, 5), inter-operator (Run 3 vs. Run 5), and inter-instrument (Run 3 vs. Run 4) comparisons were performed. Concordance was determined based on 3 cutoffs provided by the Sponsor.

### 13.2. Results

Globo-H IHC reproducibility testing results are shown in Error! Reference source not found. (as shown in FIG. 32). Concordance around 4 cutoff values (H-score=1, 15, 20, 100, and 150) was determined and are listed in Table (inter-run), Table (inter-operator), and Table (inter-instrument). Concordance ranged from 91.1%-100% for inter-run, 77.8%-100% for inter-operator, and 83.3%-100% for inter-instrument across the 5 cutoff values.

TABLE 12
Globo-H IHC Reproducibility Concordance - Inter-Run (Runs 1-5)

| | | Concordance | Concordance | Concordance | Concordance | Concordance |
|---|---|---|---|---|---|---|
| | | of H- | of H- | of H- | of H- | of H- |
| Specimen | Indication | score ≥1 | score ≥15 | score ≥20 | score ≥100 | score ≥150 |
| F00060972 | Esophageal | 100% | 100% | 100% | 100% | 100% |
| F00060983 | Esophageal | 100% | 100% | 100% | 100% | 100% |
| F00061012 | Esophageal | 100% | 80% | 60% | 100% | 100% |
| 00-14556-A5 | CRC | 100% | 100% | 100% | 100% | 100% |
| 99-9021-A4 | CRC | 100% | 100% | 100% | 100% | 100% |
| 00-8225-A4 | CRC | 100% | 100% | 100% | 100% | 100% |
| 99-4637-E1 | NSCLC | 80% | 100% | 100% | 100% | 100% |
| 99-11848-F6 | NSCLC | 100% | 100% | 100% | 100% | 100% |
| 98-17523-B2 | NSCLC | 100% | 100% | 100% | 100% | 100% |
| 92-4644-B12 | Gastric | 100% | 100% | 100% | 100% | 100% |
| 93-2141-B6 | Gastric | 100% | 60% | 60% | 100% | 100% |
| 94-2834-A6 | Gastric | 100% | 100% | 100% | 80% | 100% |
| 93-8057-40 | PDA | 100% | 100% | 100% | 100% | 100% |
| 90-4304-4A | PDA | 60% | 100% | 100% | 100% | 100% |
| F00026463 | PDA | 100% | 60% | 60% | 100% | 100% |
| 92-148-6 | HCC | 100% | 100% | 100% | 100% | 100% |
| F00084252 | HCC | 100% | 60% | 100% | 100% | 100% |
| F00083288 | HCC | 100% | 100% | 60% | 100% | 100% |

| | | | | | |
|---|---|---|---|---|---|
| Overall Concordance | 96.7% | 92.2% | 91.1% | 98.9% | 100% |

TABLE 13
Globo-H IHC Reproducibility Concordance - Inter-Operator (Runs 3 and 5)

| | | Concordance | Concordance | Concordance | Concordance | Concordance |
|---|---|---|---|---|---|---|
| | | of H- | of H- | of H- | of H- | of H- |
| Specimen | Indication | score ≥1 | score ≥15 | score ≥20 | score ≥100 | score ≥150 |
| F00060972 | Esophageal | 100% | 100% | 100% | 100% | 100% |
| F00060983 | Esophageal | 100% | 100% | 100% | 100% | 100% |
| F00061012 | Esophageal | 100% | 0% | 100% | 100% | 100% |
| 00-14556-A5 | CRC | 100% | 100% | 100% | 100% | 100% |
| 99-9021-A4 | CRC | 100% | 100% | 100% | 100% | 100% |
| 00-8225-A4 | CRC | 100% | 100% | 100% | 100% | 100% |
| 99-4637-E1 | NSCLC | 100% | 100% | 100% | 100% | 100% |
| 99-11848-F6 | NSCLC | 100% | 100% | 100% | 100% | 100% |
| 98-17523-B2 | NSCLC | 100% | 100% | 100% | 100% | 100% |
| 92-4644-B12 | Gastric | 100% | 100% | 100% | 100% | 100% |
| 93-2141-B6 | Gastric | 100% | 0% | 0% | 100% | 100% |
| 94-2834-A6 | Gastric | 100% | 100% | 100% | 100% | 100% |
| 93-8057-40 | PDA | 100% | 100% | 100% | 100% | 100% |
| 90-4304-4A | PDA | 100% | 100% | 100% | 100% | 100% |
| F00026463 | PDA | 100% | 0% | 100% | 100% | 100% |
| 92-148-6 | HCC | 100% | 100% | 100% | 100% | 100% |
| F00084252 | HCC | 100% | 0% | 100% | 100% | 100% |
| F00083288 | HCC | 100% | 100% | 0% | 100% | 100% |

| | | | | | |
|---|---|---|---|---|---|
| Overall Concordance | 100% | 77.8% | 88.9% | 100% | 100% |

TABLE 14
Globo-H IHC Reproducibility Concordance - Inter-Instrument (Runs 3 and 4)

| | | Concordance | Concordance | Concordance | Concordance | Concordance |
|---|---|---|---|---|---|---|
| | | of H- | of H- | of H- | of H- | of H- |
| Specimen | Indication | score ≥1 | score ≥15 | score ≥20 | score ≥100 | score ≥150 |
| F00060972 | Esophageal | 100% | 100% | 100% | 100% | 100% |
| F00060983 | Esophageal | 100% | 100% | 100% | 100% | 100% |
| F00061012 | Esophageal | 100% | 100% | 0% | 100% | 100% |
| 00-14556-A5 | CRC | 100% | 100% | 100% | 100% | 100% |
| 99-9021-A4 | CRC | 100% | 100% | 100% | 100% | 100% |
| 00-8225-A4 | CRC | 100% | 100% | 100% | 100% | 100% |
| 99-4637-E1 | NSCLC | 0% | 100% | 100% | 100% | 100% |
| 99-11848-F6 | NSCLC | 100% | 100% | 100% | 100% | 100% |
| 98-17523-B2 | NSCLC | 100% | 100% | 100% | 100% | 100% |
| 92-4644-B12 | Gastric | 100% | 100% | 100% | 100% | 100% |
| 93-2141-B6 | Gastric | 100% | 100% | 100% | 100% | 100% |
| 94-2834-A6 | Gastric | 100% | 100% | 100% | 100% | 100% |
| 93-8057-40 | PDA | 100% | 100% | 100% | 100% | 100% |
| 90-4304-4A | PDA | 0% | 100% | 100% | 100% | 100% |
| F00026463 | PDA | 100% | 0% | 0% | 100% | 100% |
| 92-148-6 | HCC | 100% | 100% | 100% | 100% | 100% |
| F00084252 | HCC | 100% | 100% | 100% | 100% | 100% |
| F00083288 | HCC | 100% | 100% | 0% | 100% | 100% |

| | | | | | |
|---|---|---|---|---|---|
| Overall Concordance | 88.9% | 94.4% | 83.3% | 100% | 100% |

### 13.3. Acceptability Criteria

The AVERAGE percent CV (% CV) of the results of the five slides from each specimen stained on five independent runs must be ≤20% and/or the concordance ≥85% (if a cutoff is provided) or approved by the Sponsor.

### 13.4. Conclusion

The GLOBO-H IHC inter-run reproducibility study resulted in an overall concordance of 96.7%, 92.2%, 91.1%, 98.9%, and 100% when using H-score cutoffs of ≥1, ≥15, ≥20, ≥100, and ≥150, respectively, and met the acceptability criteria. The inter-operator reproducibility study resulted in an overall concordance of 100%, 77.8%, 88.9%, 100%, and 100% when using H-score cutoffs of ≥1, ≥15, ≥20, ≥100, and ≥150, respectively. The inter-instrument reproducibility study resulted in an overall concordance of 88.9%, 94.4%, 83.3%, 100%, and 100% when using H-score cutoffs of ≥1, ≥15, ≥20, ≥100, and ≥150, respectively.

### 14. Additional Precision—Reproducibility—Inter-Operator

### 14.1. Study Setup

In order to perform the Globo H pan tumor IHC precision inter-operator study, as outlined in THE amended precision plan, 3 specimens per indication (esophageal, CRC, NSCLC, gastric, and HCC), representing the overall dynamic range of Globo H IHC expression, were selected by NeoGenomics in conjunction with the Sponsor. Where possible, these were the same specimens used in the initial precision study. Note: Three (3) specimens were selected and tested to ensure that the required “n” of 2 described in the amended study plan would be evaluable. Four (4) sections from each specimen were stained on 2 independent staining runs, on non-consecutive days, using the approved optimized Globo H IHC assay. Each run was performed by a different operator using a single instrument, with 2 replicates per specimen stained with Globo H and a single replicate per specimen stained with an isotype matched IgG negative control per run. Specimen 92-148-6 had minimal tissue remaining thus only 1 replicate was stained per run. Positive and negative batch controls, consisting of human breast cancer tissue, were included in each run and stained with the approved optimized Globo H IHC assay and a matched isotype control. The inter-operator staining structure is listed below:
Run 1: Operator 1, instrument 1 (serial sections 1&3 with Globo H)
Run 2: Operator 2, instrument 1 (serial sections 2&4 with Globo H)

IHC STAINED slides were evaluated by a NeoGenomics pathologist using the Globo H scoring criteria provided during Globo H IHC pathologist evaluation training. Concordance was calculated based on the Sponsor provided cutoff (H-score ≥15). There were 4 comparisons per specimen: serial sections 1 vs. 2, 1 vs. 4, 3 vs. 2, and 3 vs. 4.

### 14.2. Results

Globo H IHC inter-operator reproducibility results are shown in Table 15 Error! Reference source not found. (as shown in FIG. 33). Specimen F00061012 was not evaluable due to insufficient tumor content (<100 cells). Since only 1 comparison could be performed for specimen 92-148-6 due to the limited number of replicates available, this specimen was not included in the concordance assessment. Concordance around H-score cutoff ≥15 was determined and is listed in Table 16. All specimens showed concordant inter-operator results except for 2 specimens where the results were around the cut-off of 15. The variances seen for these specimens was no greater than the variances seen for other specimens, but because the “target” score was near the cut-off, these variances led to a positive/negative discordance. Overall, Inter-operator concordance ranged from 0%-100% with an average concordance of 85%.

TABLE 16
Globo H IHC Inter-Operator Concordance

| | | Run 1- | Run 1- | Run 1- | Run 1- | |
|---|---|---|---|---|---|---|
| | | Replicate | Replicate | Replicate | Replicate | Specimen |
| | | 1 vs. Run 2 | 1 vs. Run 2 | 2 vs. Run 2 | 2 vs. Run 2 | Concordance |
| | | Replicate | Replicate | Replicate | Replicate | at H- |
| Specimen | Indication | 1 | 2 | 1 | 2 | score ≥15 |
| F00060972 | Esophageal | Concordant | Concordant | Concordant | Concordant | 100% |
| F00060983 | Esophageal | Concordant | Concordant | Concordant | Concordant | 100% |
| 00-14556-A7 | CRC | Concordant | Concordant | Concordant | Concordant | 100% |
| 99-9021-A4 | CRC | Concordant | Concordant | Concordant | Concordant | 100% |
| 00-8225-A4 | CRC | Concordant | Concordant | Concordant | Concordant | 100% |
| 99-4637-E1 | NSCLC | Concordant | Concordant | Concordant | Concordant | 100% |
| 99-11848-F6 | NSCLC | Discordant | Discordant | Discordant | Discordant | 0% |
| 98-17523-B2 | NSCLC | Concordant | Concordant | Concordant | Concordant | 100% |
| 92-4644-B12 | Gastric | Discordant | Discordant | Discordant | Discordant | 0% |
| 93-2141-B6 | Gastric | Concordant | Concordant | Concordant | Concordant | 100% |
| 94-2834-A6 | Gastric | Concordant | Concordant | Concordant | Concordant | 100% |
| F00084252 | HCC | Concordant | Concordant | Concordant | Concordant | 100% |
| F00083288 | HCC | Concordant | Concordant | Concordant | Concordant | 100% |

| | |
|---|---|
| Average Concordance | 85% |

### 14.3. Acceptability Criteria

The average CONCORDANCE of the 4 replicates from each specimen stained by 2 operators must be ≥85%.

### 14.4. Conclusion

The GLOBO H inter-operator concordance was 85% and met the acceptability criteria.

### 15. Additional Precision—Reproducibility—Inter-Instrument

### 15.1. Study Setup

In order to perform the inter-instrument precision study for the pancreatic cancer indication AS described in the amended precision plan, 3 pancreatic cancer specimens, representing a range of Globo H IHC expression, were selected by NeoGenomics in conjunction with the Sponsor. These were the same pancreatic specimens used in the initial precision study. Note: Three (3) specimens were selected and tested to ensure that the required “n” of 2 described in the amended study plan would be evaluable. Four (4) sections from each specimen were stained on 2 independent staining runs, on non-consecutive days, using the approved optimized Globo H IHC assay. Each run was performed on a different instrument by a single operator, with 2 replicates per specimen stained with Globo H and a single replicate per specimen stained with an isotype matched IgG negative control per run. Positive and negative batch controls, consisting of human breast cancer tissue, were included in each run and stained with the approved optimized Globo H IHC assay and a matched isotype control. The inter-instrument staining structure is listed below:
Run 1: Operator 1, instrument 1 (serial sections 1&3 with Globo H)
Run 2: Operator 1, instrument 2 (serial sections 2&4 with Globo H)

Stained slides WERE evaluated by a NeoGenomics pathologist using the Globo H scoring criteria provided during pathology training. Concordance was calculated based on the Sponsor provided cutoff (H-score ≥20). There were 4 comparisons per specimen: serial sections 1 vs. 2, 1 vs. 4, 3 vs. 2, and 3 vs. 4.

### 15.2. Results

Globo H IHC INTER-instrument reproducibility results are shown in Table 17 (as shown in FIG. 34). Concordance around H-score cutoff ≥20 was determined and is listed in Table 18. Inter-instrument concordance was 100% for all specimens.

TABLE 18
Globo H IHC Inter-Instrument Concordance

| | | Run 1- | Run 1- | Run 1- | Run 1- | |
|---|---|---|---|---|---|---|
| | | Replicate | Replicate | Replicate | Replicate | Specimen |
| | | 1 vs. Run 2 | 1 vs. Run 2 | 2 vs. Run 2 | 2 vs. Run 2 | Concordance |
| | | Replicate | Replicate | Replicate | Replicate | at H- |
| Specimen | Indication | 1 | 2 | 1 | 2 | score ≥20 |
| 93-8057-40 | PDA | Concordant | Concordant | Concordant | Concordant | 100% |
| 90-4304-4A | PDA | Concordant | Concordant | Concordant | Concordant | 100% |
| F00026463 | PDA | Concordant | Concordant | Concordant | Concordant | 100% |

| | |
|---|---|
| Average Concordance | 100% |

### 15.3. Acceptability Criteria

The AVERAGE concordance of the 4 replicates from each specimen stained on 2 instruments must be ≥85%.

### 15.4. Conclusion

The Globo H inter-instrument concordance was 100% and met the acceptability criteria.

### 16. Reportable Range

The reportable range for Globo H IHC is H-score 0-300.

### 17. Cutoff Values

Cutoff values of H-SCORE ≥1, ≥15, ≥20, ≥100 and ≥150 were used to assess Precision and Globo H prevalence.

The Sponsor SELECTED a cutoff value of H-score ≥100 for pancreatic, CRC, gastric, HCC, NSCLC, and esophageal cancer specimens based on mechanism of action of the Globo H-targeting drugs and Globo H prevalence. A higher cutoff value with reasonable prevalence is assumed to provide a better chance to benefit patients treated with Globo H-targeting drugs.

### Example 2: Validation of Globo-H IHC for Breast Cancer Application

### 1. Purpose

The purpose of this STUDY was to develop, optimize and validate an immunohistochemistry (IHC) assay for the detection of Globo-H expression in breast cancer. Three anti-Globo H antibodies were utilized to develop and optimize a Globo-H IHC staining procedure on formalin fixed, paraffin embedded (FFPE) breast cancer specimens. The Globo-H IHC assay was validated in breast cancer by assessing accuracy, sensitivity, specificity and precision. FDA normal TMA and a breast tumor TMA were also screened using the optimized Globo-H IHC assay.

### 2. Definitions & Acronyms

| Word/Acronym | Definition |
|---|---|
| μm | Micrometer or micron |
| CRC | Colorectal cancer |
| CV | Coefficient of variance |
| FDA | Food and Drug Administration |
| FFPE | Formalin-fixed paraffin-embedded |
| Globo-H | Glycosphingolipid of the globo series |
| | with a sugar terminus resembling |
| | the blood group antigen H determinant |
| IHC | Immunohistochemistry |
| N/A or NA | Not applicable |
| TMA | Tissue micro-array |
| TNBC | Triple negative breast cancer |

### 3. Associated Reagents/Probes/Antibodies

| Name | Lot Number | Expiration | Storage Temperature | |
|---|---|---|---|---|
| Anti-Globo-H antibody, VK9, | 4306278 | MAY 2021 | 2-8° | C. |
| eBioscience (ThermoFisher, Cat# | | | | |
| 14-9700-82) | | | | |
| MCF-7; 105-EN-024 Grl-5/Grl-7 | N/A | N/A | Ambient | |
| xenograft FFPE block | | | | |
| MDA-MB-231: 105-EN-065 #1-4 | N/A | N/A | Ambient | |
| xenograft FFPE block | | | | |
| HPAC: 105-EN-051 Grl-7 | N/A | N/A | Ambient | |
| xenograft FFPE block | | | | |
| EnVision ™ FLEX, High pH, (Link) | 10129395 | JULY 2018 | 2-8° | C. |
| Kit (Dako, Cat# K8002): | | JUNE 2018 | | |
| Envision FLEX Target Retrieval | 10127384 | | | |
| Solution, High pH | | | | |
| En Vision FLEX Peroxidase- | | | | |
| Blocking Reagent | | | | |
| En Vision FLEX/HRP | | | | |
| Envision FLEX Wash Buffer 20X | | | | |
| DAB Substrate Buffer & | | | | |
| Chromogen DAB Substrate Buffer | | | | |
| & Chromogen | | | | |
| EnVision ™ FLEX Target Retrieval | 10128667 | JULY 2018 | 2-8° | C. |
| Solution, Low pH (Dako, Cat# | 10125515 | MAY 2018 | | |
| K8005) | 10123661 | APRIL 2018 | | |
| | 10123653 | FEBRUARY 2018 | | |
| Antibody Diluent with Background- | 10123238 | AUGUST 2018 | 2-8° | C. |
| Reducing Components (Dako, Cat# | 10129260 | JANUARY 2019 | | |
| S3022) | 10119758 | APRIL 2018 | | |
| EnVision ™ FLEX Hematoxylin | 10130055 | SEPTEMBER 2018 | Ambient | |
| (Dako, Cat# K8008) | 10131332 | JUNE 2018 | | |
| Negative control antibody: Mouse | IX2416121 | AUGUST 2018 | −10 to −30° | C. |

| | | | |
|---|---|---|---|
| IgG Isotype Control (R&D Systems, | | | |
| Cat# MAB002) | | | |
| Deionized Water | N/A | N/A | Ambient |
| 100% Alcohol | N/A | N/A | Ambient |
| 95% Alcohol | N/A | N/A | Ambient |
| Xylene | N/A | N/A | Ambient |

### 4. Associated Equipment

| Name | Model |
|---|---|
| Microtome | Leica RM2235 or equivalent |
| Flotation Bath | TBS TFBL or equivalent |
| Drying oven | Biocare 10-180Aer or equivalent |
| Antigen Retrieval Chamber | Dako PT Link |
| Slide stainer | Dako Link 48 |
| Linear stainer | Leica Autostainer XL or equivalent |
| Cover slipper | Tissue-Tek Film 4740 or equivalent |

### 5. Specimen Requirements

### 5.1 Storage Time and Temperature:

FFPE TISSUE blocks were sectioned at 4-5 μm and mounted on positively charged slides. Slides were air-dried and stored throughout the duration of the study at room temperature.

5.2 Unacceptable Specimens: Specimens that are not FFPE.

### 6. Specimen De-Identification

### 6.1 Source of Specimen:

Breast cancer specimens (n=65) from a Phase II OBI-822-001 Clinical Study were provided by the Sponsor as FFPE tissue sections on positively charged slides. Breast cancer specimens (n=20) and a breast cancer TMA were provided by NeoGenomics as FFPE tissue blocks from a qualified vendor and the AGI tissue bank. Cell line control blocks were provided by the Sponsor.

### 6.2 Description of De-Identification Process:

Specimens were labeled with identifiers that NeoGenomics cannot trace back to the patient.

### Study Design

### 7. Globo-H Assay Controls and IHC Scoring

7.1 Batch control tissues used in each staining run consisted of a known positive cell line block (HPAC), confirmed by LC-MS/MS, and a human breast cancer tissue (from AGI tissue bank) containing elements with positive and negative Globo-H expression; these controls were all stained with Globo-H antibody and negative control antibody. Representative images of the human breast cancer tissue control are shown in FIGS. 15A to 15F.

FIGS. 15A to 15F show the Globo-H IHC staining of the breast cancer control tissue. FIG. 15A shows Globo-H staining in tumor region, FIG. 15B shows high magnification of Globo-H staining in tumor region, FIG. 15C shows Globo-H staining in non-tumor region, FIG. 15D shows high magnification of Globo-H staining in non-tumor region, FIG. 15E shows Negative control antibody staining in tumor region, and FIG. 15F shows Negative control antibody staining in normal breast region.

7.2 Semi-quantitative Globo-H pathologist evaluation criteria were established and documented using the optimized Globo-H IHC stained specimens (breast cancer specimens included in the development/optimization phase).

7.3 Globo-H IHC stained slides were evaluated by a NeoGenomics pathologist using a brightfield microscope.

7.4 Globo-H IHC expression was assessed in tumor cells. The percent of tumor cells with membrane and/or cytoplasmic Globo-H expression was recorded at each staining intensity (0, 1+, 2+, and 3+), FIGS. 16A to 16E. The intensity of the Globo-H expression was reported as 0 for no detectable staining; 1+ for translucent or low level staining; 2+ for moderate or opaque staining; and 3+ for strong or solid staining. The H-score values range from 0 to 300 and is calculated with the following formula: H-score=[(% of Tumor Cell Membrane/Cytoplasmic at 1+)×1+(% of Tumor Cell Membrane/Cytoplasmic at 2+)×2+(% of Tumor Cell Membrane/Cytoplasmic at 3+)×3]

FIGS. 16A to 16E show the Globo-H IHC staining intensities in a breast cancer specimen. FIG. 16A shows Heterogeneous Globo-H staining of breast carcinoma cells, FIG. 16B shows intense staining, FIG. 16C shows moderate staining, FIG. 16D shows weak staining, and FIG. 16E shows no staining.

7.5 the Pathologist Also Recorded if Nuclear Expression was Observed.

| | | | | |
|---|---|---|---|---|
| Specimen | Negative | Tumor Cell Membrane/Cytoplasmic | Nuclear | Comments |
| ID | Control | Percent Positive (%) | Staining | |

| | | | | |
|---|---|---|---|---|
| 0 | 1+ | 2+ | 3+ | Observed? |
| | | | | Y/N |

7.6 A semi-quantitative H-score assessment of Globo-H positive cells was calculated and reported.

The standard H-score methodology was used as an approach to measuring Globo-H expression in a semi-quantitative fashion. Based on the cell membrane and cytosolic translocating feature of Globo-H, the cells with positive signal on the cell membrane or cytosolic are considered positive staining. This scoring methodology allows the assessment of Globo-H immunoexpression that takes into account both the percentage of cells expressing this antigen and the intensity (level of expression) per cell.

The score is obtained by reviewing specimens based on a 0-3+ intensity gradient and determining the percentage of cells expressing the marker at each of the 4 levels of intensity (0, 1+ (weakly staining), 2+ (moderately staining), 3+ (strongly staining) using the formula: H score=[(% of Tumor Cell Membrane and/or Cytoplasmic at 1+)×1+(% of Tumor Cell Membrane and/or Cytoplasmic at 2+)×2+(% of Tumor Cell Membrane and/or Cytoplasmic at 3+)×3], yielding a dynamic range of 0-300.

For Globo-H, the specimen is assessed for the percentage of Globo-H IHC staining of tumor cells (primarily cytoplasmic staining) across the 0-3+ intensity levels, and the H-score is calculated accordingly, as described above.

7.7 The Sponsor assessed H-score cutoffs of ≥1, ≥15, and ≥20. Prevalence and concordance is reported using the above cutoff values.

### 8. Optimization and TMA Screening

### 8.1 Study Setup

Globo-H IHC Development and Optimization: Three OBI anti-Globo H antibody clones (VK9: OBI-042, 2C2: OBI-007 and 2F8: OBI-016) and a commercially available anti-Globo H antibody clone (VK9) were evaluated on 3 staining platforms (Dako Link 48, Leica Bond III and Ventana Benchmark Ultra) using positive and negative control cell lines (provided by Sponsor, Globo-H contents estimation by LC-MS/MS) and breast cancer FFPE specimens from NeoGenomics' archives. Globo-H IHC assays were optimized by adjusting antibody concentrations, incubation times, antigen retrieval methodologies, blocking reagents, and detection systems, per staining platform and clone, under the guidance of a qualified pathologist. Globo-H IHC staining was qualitatively evaluated to assess specific and background staining using a manual brightfield microscope. Globo-H IHC stained specimens were scanned at 20× objective magnification using an Aperio scanner. The degree of magnification was determined after review and confirmation that subcellular compartments (nuclear, cytoplasmic, membrane) can be adequately defined and evaluated. Digitized images of optimized staining, per clone, were reviewed with the Sponsor, and a single optimized Globo-H IHC assay was selected and approved for the Globo-H (VK9) antibody. Pathology evaluation criteria, as outlined in Section 8, were established by NeoGenomics in conjunction with the Sponsor.

FDA normal TMA and tumor TMA screening: An FDA normal TMA (37 normal tissues, 2-3 unique donors each) was stained using the Globo-H IHC assay selected for VK9 antibody. A single Globo-H IHC staining procedure was selected and approved by the Sponsor after reviewing images of the normal TMA Globo-H IHC stained slides. The Globo-H IHC stained FDA normal TMA was evaluated by a qualified pathologist. The approved optimized Globo-H IHC procedure was also used to stain a breast cancer TMA. The breast cancer TMA was evaluated by a qualified pathologist using the Globo-H evaluation criteria established previously (above). The prevalence results are reported as found using cutoff values requested by the Sponsor.

### 8.2 Results

### Globo-H IHC Development and Optimization:

Three proprietary Globo-H clones from the Sponsor and the commercially available Globo-H clone VK9 (OBI-042) antibody were tested on 3 automated IHC platforms. Staining on the Ventana Benchmark Ultra did not yield satisfactory staining for any of the 3 antibodies when using cell line control material. Globo-H clone 2C2 (OBI-007) and 2F8 (OBI-016) antibodies did not yield specific or clean staining on either the Leica Bond III or Dako Link 48 platforms. OBI-042 demonstrated specific Globo-H staining on both the Leica Bond III and Dako Link 48 platforms. The Dako platform and detection system resulted in stronger Globo-H staining then did the Leica Bond III platform and detection system. Antibody OBI-042 showed stronger overall staining of Globo-H compared to other antibodies, but the background staining interfered with pathology assessment of specific staining.

Based on optimization efforts with 3 Globo-H antibodies, the Globo-H mouse monoclonal antibody (VK9) on the Dako Link 48 platform was selected. The optimized assay is summarized below.

Briefly, tissues were sectioned at 4-5 μm, mounted on positively charged slides, and allowed to air-dry. Slides were then heated in a 58° C. drying oven for 60 minutes. Deparaffinization and epitope retrieval was performed using En Vision FLEX Low PH TRS (Agilent) in the Dako PT Link module for 40 minutes at 97° C. Slides were allowed to cool to 65° C., then removed from the PT Link and placed in a FLEX wash buffer bath for 5 minutes. Reagents from the En Vision™ FLEX, High pH Kit were used to perform the following steps on the Dako Link 48 IHC platform. In between each step, slides were rinsed with deionized water or FLEX wash buffer, as noted in Table 1. Slides were treated with En Vision FLEX Peroxidase Block for 5 minutes followed by Globo-H primary antibody (10 μg/mL, 1:50 in Dako Background Reducing Diluent) incubation for 60 minutes. Visualization was achieved with En Vision FLEX HRP for 30 minutes and EnVision FLEX DAB+ for 10 minutes. Slides were counterstained on the Dako Link 48 using EnVision FLEX Hematoxylin for 5 minutes, then removed from the instrument. Slides were dehydrated using a series of graded alcohol and cleared with xylene prior to being coverslipped using a Tissue-Tek automated film coverslipper.

Images of the optimized protocol in breast cancer tissue are displayed in FIGS. 17A to 17D. The processing steps of the optimized Globo-H IHC assay are listed in Table 19 and diagramed in FIG. 18. A schematic image of the detection system binding is displayed in FIG. 19. The Globo-H IHC assay was validated in breast cancer, assessing accuracy, sensitivity, specificity, and intra-run and inter-run reproducibility in the following sections.

TABLE 19
Globo-H Optimized IHC Protocol on the Dako LINK 48 - Clone VK9

| | Incubation | | Dispense | | |
|---|---|---|---|---|---|
| Reagent | Time | | Volume | Temperature | |
| Tissue Sectioning (4-5 μm) | N/A | | N/A | Ambient | |
| Labeling and Storage | N/A | | N/A | Ambient | |
| Bake Slides | 60 | min | N/A | 58-62° | C. |
| Print Dako Labels | N/A | | N/A | Ambient | |
| PT Link Antigen | 40 | min | N/A | 97° | C. |

| Retrieval - Low pH TRS | | | | |
|---|---|---|---|---|
| Wash Buffer | 5 | min | N/A | Ambient |
| Wash Buffer Rinse | 0 | min | N/A | Ambient |
| Peroxide Block | 5 | min | 300 μl | Ambient |
| | | | (150 μl × 2 zones) | |
| Wash Buffer Rinse | 0 | min | N/A | Ambient |
| Globo-H Antibody | 60 | min | 300 μl | Ambient |
| (10 μg/mL, 1:50) | | | (150 μl × 2 zones) | |
| Wash Buffer Rinse | 0 | min | N/A | Ambient |
| FLEX HRP | 30 | min | 300 μl | Ambient |
| | | | (150 μl × 2 zones) | |
| Wash Buffer Rinse | 0 | min | N/A | Ambient |
| Wash Buffer | 5 | min | N/A | Ambient |
| FLEX DAB+ | 10 | min | 300 μl | Ambient |
| | | | (150 μl × 2 zones) | |
| Wash Buffer Rinse | 0 | min | N/A | Ambient |
| FLEX Hematoxylin | 5 | min | 300 μl | Ambient |
| | | | (150 μl × 2 zones) | |
| Deionized Water | 0 | sec | N/A | Ambient |
| Wash Buffer Rinse | 5 | min | N/A | Ambient |
| Deionized Water | 0 | sec | N/A | Ambient |
| Unload from Dako, | 7 | minutes | N/A | ambient |

| | | | |
|---|---|---|---|
| Dehydrate, and Clear | | | |
| Coverslip | NA | N/A | ambient |

Schematic of two-step polymer-based En Vision FLEX IHC detection system. Antibody binding to antigen, followed by secondary antibody with dextran polymer containing up to 100 HRP molecules.

### FDA Normal TMA and Tumor TMA Screening:

Results from the FDA normal tissue TMA are listed in FIG. 37. The observed Globo-H IHC expression by tissue type is summarized in the Specificity section of the validation.

Results from screening a breast cancer TMA are listed in FIG. 38. The observed Globo-H IHC expression in the breast cancer TMA is summarized in Table 20. Representative images are shown in FIGS. 20A to 20D.

TABLE 20
Summary of Globo-H IHC Expression in a Breast Cancer TMA

| Tumor | H- | H- | Positive | H- | H- | Positive | H- | H- | Positive |
|---|---|---|---|---|---|---|---|---|---|
| Indication | score <1 | score ≥1 | Prevalence | score <15 | score ≥15 | Prevalence | score <20 | score ≥20 | Prevalence |
| Breast | 15 | 35 | 70% | 33 | 17 | 34% | 35 | 15 | 30% |

### 8.3 Acceptability Criteria

Review and approval of the optimized Globo-H IHC assay. TMA results are reported as found.

### 8.4 Conclusion

The optimized Globo-H (clone VK9) IHC assay yielded specific Globo-H IHC signal with minimal background and was approved by the Sponsor.

### 9. Accuracy

### 9.1 Study Setup

HPAC and SK-BR3 cell line blocks, provided by the Sponsor, were stained and evaluated by NeoGenomics using the approved optimized Globo-H IHC and pathologist evaluation procedures. Results were compared to Sponsor data by testing the same cell lines by mass spectrometry.

Additionally, 85 breast cancer specimens and a breast cancer TMA were stained and evaluated using the approved optimized Globo-H IHC and pathologist evaluation procedures. A negative control antibody was included for each specimen when slides were available. An H&E stain was performed on each specimen to aid in pathologist evaluation (for morphological/histological reference). Batch control tissues used in each staining run consisted of a known positive cell line block (HPAC), confirmed by LC-MS/MS, and a human breast cancer tissue (from AGI tissue bank) containing elements with positive and negative Globo-H expression; these controls were all stained with Globo-H antibody and negative control antibody. Sixty-five (65) of the specimens were previously characterized breast cancer specimens from a Phase II OBI-822-001 Clinical Study provided by Sponsor. The previously characterization method was an exploratory analysis generated by Academia Sinica (Hung et al. 2015)⁽⁶⁾. Twenty (20) uncharacterized specimens were provided by NeoGenomics. The Globo-H positive and negative prevalence for the 65 pre-characterized breast specimens was determined based on the Globo-H IHC status (positive/negative) established using the cutoff defined by the Sponsor which was correlated to clinical outcome of the Phase II OBI-822-001 Clinical Study. The number of observed Globo-H positive and negative specimens was compared to the number of expected positive and negative specimens based on Sponsor data using 2×2 contingency table(s).

### 9.2 Statistical and Algorithmic Calculations

$$Accuracy = [ \text{} ( \# True positive + \# True Negative ) \text{} \text{/}$$

$$( \# True Positive + \text{} \# False Positive + \# False Negative +$$

$$\# True Negative ) ] \times 100 \%$$

### 9.3 Results

Results from staining HPAC and SK-BR3 cell line blocks with the optimized and approved Globo-H IHC assay are listed in Table 21. Representative images of the cell line blocks are displayed in FIGS. 21A to 21B. Globo-H expression was observed in the HPAC cell line (FIG. 21A), but not in the SK-BR3 cell line (FIG. 21B), consistent with Sponsor findings.

TABLE 21
Globo-H IHC Expression in - Cell Line Controls

| | | Tumor Cell | | | | | | Nuclear |
|---|---|---|---|---|---|---|---|---|
| | | Membrane/Cytoplasmic | | | | | | Staining |
| Specimen | Negative | Percent Positive (%) | | | | Percent | | Observed? |
| ID | Control | 0 | 1+ | 2+ | 3+ | Positive | H-Score | Y/N |
| OBI-A (HPAC) | 0 | 95 | 0 | 3 | 2 | 5 | 12 | N |
| OBI-E (SK-BR3) | 0 | 100 | 0 | 0 | 0 | 0 | 0 | N |

Of the 65 previously tested breast cancer specimens, 61 specimens were evaluable after being stained using the approved optimized Globo-H IHC assay. Results from staining 85 breast cancer specimens are listed in FIG. 39. Prior testing conducted in Academia Sinica used OBI-042 as primary antibody and used an H-score cutoff of ≥80 to categorize specimens as Globo-H positive. The prior testing data was compared to NeoGenomics results utilizing an H-score cutoff ≥15 to categorize specimens as Globo-H positive. Table 22 is a contingency table comparing the 2 data sets.

TABLE 22
Comparison of 61 Breast Cancer Specimens Assessed by NeoGenomics to
Historical Data Assessed by Academia Sinica Provided by the Sponsor

| | | Academia Sinica (Reference) - | | | |
|---|---|---|---|---|---|
| | | Previous Globo-H Results - | | | |
| | | Cutoff H-score ≥80 | | | |
| | | Expected Globo-H | Expected Globo-H | | |
| | | Positive | Negative | Total | |
| NeoGenomiics | Actual Globo-H | 16 | 10 | 26 | (42.6%) |
| Globo-H | Positive | (True Positive) | (False Positive) | | |
| Results - | Actual Globo-H | 1 | 34 | 35 | (57.4%) |
| Cutoff | Negative | (False Negative) | (True Negative) | | |
| H-score ≥15 | Total | 17 (27.9%) | 44 (72.1%) | 61 | (100%) |

Accuracy = (16 + 34)/(16 + 10 + 1 + 34) × 100% = 82.0%

### 9.4 Acceptability Criteria

Accuracy of tested specimens compared to Sponsor data is ≥85% or approved by the Sponsor.

### 9.5 Conclusion

Results from IHC staining of HPAC (positive) and SK-BR3 (negative) cell lines was consistent with Sponsor data from mass spectrometry. Using the data of the same specimens conducted by Academia Sinica as reference, the accuracy of the Globo-H IHC assay is 82.0% and was approved by the Sponsor.

### 10. Sensitivity

### 10.1 Study Setup

Results from the accuracy study in breast cancer specimens (2×2 contingency table, Table 22) were used to determine Globo-H IHC assay sensitivity.

### 10.2 Statistical and Algorithmic Calculations

$$Sensitivity = [ \# True Positive / ( \# True Positive + \# False Negative ) ] \times 100$$

### 10.3 Results

Data from Table 22 were used to calculate the sensitivity of the Globo-H IHC assay.

$$Sensitivity = [ 16 / ( 16 + 1 ) ] \times 100 \% = 94.1 \%$$

Representative images of breast cancer specimens stained with the approved optimized Globo-H IHC assay are displayed in FIGS. 22A to 22F.

Breast Cancer Specimens are: FIG. 22A: U02-1386 (H-score 0), FIG. 22B: K03-1349 (H-score 15), FIG. 22C: K02-1451 (H-score 20), FIG. 22D: U01-1404 (H-score 50), FIG. 22E: K04-1227 (H-score 105), and FIG. 22F: H02-1143 (H-score 220).

### 10.4 Acceptability Criteria

Sensitivity of tested specimens compared to Sponsor data is ≥85% or approved by the Sponsor.

### 10.5 Conclusion

The sensitivity of the Globo-H IHC assay is 94.1% and met the acceptability criteria.

### 11. Specificity

### 11.1 Study Setup

Results from the accuracy study in breast cancer specimens (2×2 contingency table, Table 22) were used to determine Globo-H IHC assay specificity.

Note: the results from screening the FDA normal TMA with the approved optimized Globo-H IHC assay, included in the optimization phase above, are included as part of the Specificity evaluation. The data is reported as found.

### 11.2 Statistical and Algorithmic Calculations

$$Specificity = [ \text{} \# True Negative \text{/}$$

$$\text{} ( \# True Negative + \# False Positive ) ] \times \text{} 100 \text{} \%$$

### 11.3 Results

Data from Table 22 were used to calculate the specificity of the Globo-H IHC assay. A summary of Globo-H IHC expression by normal tissue type is summarized in Table 23 (as shown in FIG. 35).

$$Specificity = [ 34 / ( 34 + 10 ) ] \times 100 \% = 77.3 \%$$

### 11.4 Acceptability Criteria

Specificity of tested specimens compared to Sponsor data is ≥85% or approved by the Sponsor.

### 11.5 Conclusion

The specificity of the Globo-H IHC assay is 77.3% and was approved by the Sponsor.

### 12. Precision—Repeatability

### 12.1 Study Setup

Seven breast cancer specimens representing a range of Globo-H IHC expression, with at least 2 around the cutoff, were selected by NeoGenomics in conjunction with the Sponsor for this study. Five sections from each specimen were cut and stained on one run, using the approved optimized Globo-H IHC assay. An additional slide per specimen was stained using a negative control antibody. Slides were stained on the same day using the same operator, equipment, and reagents. Batch control tissues for repeatability consisted of a known positive cell line block (HPAC), confirmed by LC-MS/MS, and a human breast cancer tissue (from AGI tissue bank) containing elements with positive and negative Globo-H expression; these controls were stained with Globo-H antibody and negative control antibody. Stained slides were evaluated by a NeoGenomics pathologist using the scoring criteria established in Section 8. Concordance was determined based on 3 cutoffs provided by the Sponsor.

### 12.2 Results

Globo-H IHC repeatability testing results are shown in Table 24. Concordance around 3 cutoff values (H-score=1, 15, and 20) was determined and are listed in Table 25.

TABLE 24
Globo-H IHC Staining Results from the Repeatability Study

| Specimen | Staining | Negative | | | | | | Total % | |
|---|---|---|---|---|---|---|---|---|---|
| ID | Date | Control | Slide | 0 | 1+ | 2+ | 3+ | Positive | H-Score |
| 98-16027-A5 | 5 DEC. 2017 | 0 | 1 | 99 | 1 | 0 | 0 | 1 | 1 |
| | | | 2 | 99 | 0 | 0 | 1 | 1 | 3 |
| | | | 3 | 99 | 1 | 0 | 0 | 1 | 1 |
| | | | 4 | 99 | 1 | 0 | 0 | 1 | 1 |
| | | | 5 | 99 | 1 | 0 | 0 | 1 | 1 |
| F00088013 | 5 DEC. 2017 | 0 | 1 | 75 | 10 | 10 | 5 | 25 | 45 |
| (TNBC) | | | 2 | 70 | 10 | 10 | 10 | 30 | 60 |
| | | | 3 | 70 | 10 | 10 | 10 | 30 | 60 |
| | | | 4 | 70 | 10 | 10 | 10 | 30 | 60 |
| | | | 5 | 70 | 10 | 10 | 10 | 30 | 60 |
| F00088012 | 5 DEC. 2017 | 0 | 1 | 90 | 5 | 5 | 0 | 10 | 15 |
| (TNBC) | | | 2 | 90 | 5 | 5 | 0 | 10 | 15 |
| | | | 3 | 90 | 5 | 5 | 0 | 10 | 15 |
| | | | 4 | 90 | 5 | 5 | 0 | 10 | 15 |
| | | | 5 | 90 | 5 | 5 | 0 | 10 | 15 |
| 99-9074-A5 | 5 DEC. 2017 | 0 | 1 | 95 | 3 | 2 | 0 | 5 | 7 |
| | | | 2 | 95 | 3 | 2 | 0 | 5 | 7 |
| | | | 3 | 95 | 3 | 2 | 0 | 5 | 7 |
| | | | 4 | 90 | 5 | 5 | 0 | 10 | 15 |
| | | | 5 | 95 | 3 | 2 | 0 | 5 | 7 |
| F00060899 | 5 DEC. 2017 | 0 | 1 | 95 | 2 | 3 | 0 | 5 | 8 |
| (TNBC) | | | 2 | 95 | 2 | 3 | 0 | 5 | 8 |
| | | | 3 | 95 | 2 | 3 | 0 | 5 | 8 |
| | | | 4 | 95 | 2 | 3 | 0 | 5 | 8 |
| | | | 5 | 95 | 2 | 3 | 0 | 5 | 8 |
| 00-3840-A6 | 5 DEC. 2017 | 0 | 1 | 10 | 0 | 0 | 0 | 0 | 0 |
| | | | 2 | 10 | 0 | 0 | 0 | 0 | 0 |
| | | | 3 | 10 | 0 | 0 | 0 | 0 | 0 |
| | | | 4 | 10 | 0 | 0 | 0 | 0 | 0 |
| | | | 5 | 10 | 0 | 0 | 0 | 0 | 0 |
| F00088015 | 5 DEC. 2017 | 0 | 1 | 10 | 0 | 0 | 0 | 0 | 0 |
| (TNBC) | | | 2 | 10 | 0 | 0 | 0 | 0 | 0 |
| | | | 3 | 10 | 0 | 0 | 0 | 0 | 0 |
| | | | 4 | 10 | 0 | 0 | 0 | 0 | 0 |
| | | | 5 | 10 | 0 | 0 | 0 | 0 | 0 |

TABLE 25
Globo-H IHC Repeatability Concordance

| | Concordance of | Concordance of | Concordance of |
|---|---|---|---|
| Specimen | H-score ≥1 | H-score ≥15 | H-score ≥20 |
| 98-16027-A5 | 100% | 100% | 100% |
| F00088013 | 100% | 100% | 100% |
| F00088012 | 100% | 100% | 100% |
| 99-9074-A5 | 100% | 80% | 100% |
| F00060899 | 100% | 100% | 100% |
| 00-3840-A6 | 100% | 100% | 100% |
| F00088015 | 100% | 100% | 100% |
| Overall Concordance | 100% | 97.1% | 100% |

### 12.3 Acceptability Criteria

The average percent CV (% CV) of the results of the five slides from each specimen stained on one run must be ≤20% and/or the concordance ≥85% (if a cutoff is provided) or approved by the Sponsor.

### 12.4 Conclusion

The Globo-H IHC repeatability study resulted in overall concordance of 100%, 97.1%, and 100% using H-score cutoffs of ≥1, ≥15, and ≥20, respectively. These values met the acceptability criteria.

### 13. Precision—Reproducibility

### 13.1 Study Setup

Seven breast cancer specimens representing a range of Globo-H IHC expression were selected by NeoGenomics in conjunction with the Sponsor for this study. These were the same specimens from repeatability. Five sections each specimen were stained on 5 independent staining runs, on non-consecutive days using the approved optimized Globo-H IHC assay. A negative control reagent was included for each specimen on each run. Batch control tissues used in each reproducibility staining run consisted of a known positive cell line block (HPAC), confirmed by LC-MS/MS, and a human breast cancer tissue (from AGI tissue bank) containing elements with positive and negative Globo-H expression; these controls were all stained with Globo-H antibody and negative control antibody. Slides from repeatability were used as one of the inter-runs. Inter operator and inter-instrument comparisons were integrated into this procedure. The staining structure is listed below:
Run 1 was extracted from the Repeatability run above (operator 1, instrument 1).
Run 2: operator 1, instrument1.
Run 3: operator 1, instrument1.
Run 4: operator 1, instrument 2.
Run 5: operator 2, instrument 1

Stained slides were evaluated by a NeoGenomics pathologist using the scoring criteria in Section 8. Inter-run (Runs 1, 2, 3, 4, 5), inter-operator (Run 3 vs. Run 5), and inter-instrument (Run 3 vs. Run 4) comparisons were performed. Concordance was determined based on 3 cutoffs provided by the Sponsor.

### 13.2 Results

Globo-H IHC reproducibility testing results are shown in Table 26 (as shown in FIG. 36). Concordance around 3 cutoff values (H-score=1, 15, and 20) was determined and are listed in Table 27 (inter-run), Table 28 (inter-operator), and Table 29 (inter-instrument). Concordance ranged from 91.4%-100% for inter-run, 85.7%-100% for inter-operator, and 71.4%-100% for inter-instrument across the 3 cutoff values.

TABLE 27
Globo-H IHC Reproducibility Concordance - Inter-Run (Runs 1-5)

| | Concordance of | Concordance of | Concordance of |
|---|---|---|---|
| Specimen | H-score ≥1 | H-score ≥15 | H-score ≥20 |
| 98-16027-A5 | 100% | 100% | 100% |
| F00088013 | 100% | 100% | 100% |
| F00088012 | 100% | 100% | 80% |
| 99-9074-A5 | 100% | 60% | 100% |
| F00060899 | 100% | 80% | 60% |
| 00-3840-A6 | 100% | 100% | 100% |
| F00088015 | 100% | 100% | 100% |
| Overall Concordance | 100% | 91.4% | 91.4% |

TABLE 28
Globo-H IHC Reproducibility Concordance -
Inter-Operator (Runs 3 and 5)

| | Concordance of | Concordance of | Concordance of |
|---|---|---|---|
| Specimen | H-score ≥1 | H-score ≥15 | H-score ≥20 |
| 98-16027-A5 | 100% | 100% | 100% |
| F00088013 | 100% | 100% | 100% |
| F00088012 | 100% | 100% | 0% |
| 99-9074-A5 | 100% | 100% | 100% |
| F00060899 | 100% | 100% | 100% |
| 00-3840-A6 | 100% | 100% | 100% |
| F00088015 | 100% | 100% | 100% |
| Overall Concordance | 100% | 100% | 85.7% |

TABLE 29
Globo-H IHC Reproducibility Concordance -
Inter-Instrument (Runs 3 and 4)

| | Concordance of | Concordance of | Concordance of |
|---|---|---|---|
| Specimen | H-score ≥1 | H-score ≥15 | H-score ≥20 |
| 98-16027-A5 | 100% | 100% | 100% |
| F00088013 | 100% | 100% | 100% |
| F00088012 | 100% | 100% | 0% |
| 99-9074-A5 | 100% | 0% | 100% |
| F00060899 | 100% | 100% | 0% |
| 00-3840-A6 | 100% | 100% | 100% |
| F00088015 | 100% | 100% | 100% |
| Overall Concordance | 100% | 85.7% | 71.4% |

### 13.3 Acceptability Criteria

The average percent CV (% CV) of the results of the five slides from each specimen stained on one run must be ≤20% and/or the concordance ≥85% (if a cutoff is provided) or approved by the Sponsor.

### 13.4 Conclusion

The Globo-H IHC reproducibility study resulted in an overall concordance of 100%, 91.4%, and 91.4% when using H-score cutoffs of ≥1, ≥15, and ≥20, respectively. These values met the acceptability criteria.

### 14. Inter-Pathologist Concordance

### 14.1 Study Setup

A second pathologist scored the Precision study slides (repeatability and reproducibility) per Sponsor request. Results from the second pathologist were compared to results from the original pathologist and assessed for concordance at H-score cutoffs of ≥1, ≥15, and ≥20.

### 14.2 Results

Scores from the second pathologist are listed in FIG. 40 and FIG. 41. The concordance at each H-score cutoff is summarized in Table 30, and comparison data is provided in FIG. 42. Overall concordance between the 2 pathologists using H-score cutoffs of ≥1, ≥15, and ≥20 was 98.4%, 79.4%, and 93.7%, respectively.

TABLE 30
Inter-Pathologist Concordance of Precision Study Slides

| | Concordance H-score ≥1 | | | Concordance H-score ≥15 | | | Concordance H-score ≥20 | | |
|---|---|---|---|---|---|---|---|---|---|
| Precision | | | #Y/(#Y + | | | #Y/(#Y + | | | #Y/(#Y + |
| Study | # N | #Y | #N) | # N | #Y | #N) | # N | #Y | #N) |
| Repeatability | 1 | 34 | 97.1% | 7 | 28 | 80.0% | 0 | 35 | 100.0% |
| Reproducibility | 0 | 28 | 100.0% | 6 | 22 | 78.6% | 4 | 24 | 85.7% |
| Overall | 1 | 62 | 98.4% | 13 | 50 | 79.4% | 4 | 59 | 93.7% |

### 14.3 Acceptability Criteria

Results are reported as found.

### 14.4 Conclusion

The overall concordance of scoring precision study slides by 2 pathologists was 98.4%, 79.4%, and 93.7% using H-score cutoffs of ≥1, ≥15, and ≥20, respectively.

### 15. Reportable Range

The reportable range for Globo-H IHC is H-score 0-300.

### 16. Cutoff Values

A cutoff value of H-score >15 was used for Accuracy, Sensitivity, and Specificity determination. Cutoff values of H-score ≥1, ≥15, and ≥20 were used to assess Precision.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

APPENDIX 1
Globo-H IHC Results of 154 Tumor Specimens (29 CRC, 24 Esophageal,
25 Gastric, 23 HCC, 29 NSCLC, and 24 Pancreatic)

| | | | | | | | | Nuclear | |
|---|---|---|---|---|---|---|---|---|---|
| | | | | | | Total | | Staining | |
| Specimen | | Percentage of Tumor Cells | | | | Percent | H- | Observed? | |
| ID | Indication | 0 | 1+ | 2+ | 3+ | Positive | score | Y/N | Comments |
| 00-12530-A8 | CRC | 100 | 0 | 0 | 0 | 0 | 0 | N | Rare, <1% |
| 00-8225-A4 | CRC | 100 | 0 | 0 | 0 | 0 | 0 | N | |
| 00-8307-B1 | CRC | 100 | 0 | 0 | 0 | 0 | 0 | N | <1% |
| 98-15598-A1 | CRC | 100 | 0 | 0 | 0 | 0 | 0 | N | |
| 98-7755-C1 | CRC | 100 | 0 | 0 | 0 | 0 | 0 | N | <1% |
| 98-8037-A1 | CRC | 100 | 0 | 0 | 0 | 0 | 0 | N | <1% |
| 99-1138-B5 | CRC | 100 | 0 | 0 | 0 | 0 | 0 | N | |
| 99-1160-B5 | CRC | 100 | 0 | 0 | 0 | 0 | 0 | N | <1% |
| 99-15594-A4 | CRC | 100 | 0 | 0 | 0 | 0 | 0 | N | |
| 99-15605-A8 | CRC | 100 | 0 | 0 | 0 | 0 | 0 | N | |
| 99-1722-C2 | CRC | 100 | 0 | 0 | 0 | 0 | 0 | N | |
| 99-9014-A9 | CRC | 100 | 0 | 0 | 0 | 0 | 0 | N | |
| 99-1712-B1 | CRC | 99 | 1 | 0 | 0 | 1 | 1 | N | |
| 99-15584-A3 | CRC | 99 | 0 | 1 | 0 | 1 | 2 | N | Edge artifact |
| 99-4730-A9 | CRC | 95 | 0 | 5 | 0 | 5 | 10 | N | Granular |
| 00-14556-A5 | CRC | 95 | 0 | 2 | 3 | 5 | 13 | N | |
| 99-1766-A2 | CRC | 65 | 35 | 0 | 0 | 35 | 35 | N | Granular |
| 00-14592-A6 | CRC | 65 | 30 | 5 | 0 | 35 | 40 | N | |
| 99-5565-A5 | CRC | 70 | 10 | 10 | 10 | 30 | 60 | N | Granular |
| 98-7733-E3 | CRC | 60 | 20 | 10 | 10 | 40 | 70 | Y | Rare |
| 00-8213-B2 | CRC | 50 | 25 | 0 | 25 | 50 | 100 | N | |
| 99-4748-A2 | CRC | 40 | 20 | 10 | 30 | 60 | 130 | Y | Rare |
| 00-14381-A1 | CRC | 30 | 20 | 10 | 40 | 70 | 160 | Y | |
| 00-8344-A5 | CRC | 25 | 20 | 15 | 40 | 75 | 170 | N | |
| 99-4750-A1 | CRC | 30 | 10 | 10 | 50 | 70 | 180 | N | Granular |
| 00-14571- | CRC | 20 | 20 | 10 | 50 | 80 | 190 | Y | Edge artifact on |
| A11 | | | | | | | | | isotype control. |
| 99-5657-B5 | CRC | 10 | 30 | 0 | 60 | 90 | 210 | N | Granular |
| 98-15614-A3 | CRC | 25 | 0 | 0 | 75 | 75 | 225 | N | Granular |
| 99-9021-A4 | CRC | 25 | 0 | 0 | 75 | 75 | 225 | N | |
| 93-10896-1G | CRC | N/A | N/A | N/A | N/A | N/A | N/A | N/A | No tumor identified |
| F00016254 | Esophageal | 100 | 0 | 0 | 0 | 0 | 0 | N | |
| F00016256 | Esophageal | 100 | 0 | 0 | 0 | 0 | 0 | N | <1% |
| F00061012 | Esophageal | 100 | 0 | 0 | 0 | 0 | 0 | N | |
| F12611.2bc | Esophageal | 100 | 0 | 0 | 0 | 0 | 0 | N | |
| F31605.Nd | Esophageal | 100 | 0 | 0 | 0 | 0 | 0 | N | |
| F31643.1Ba | Esophageal | 100 | 0 | 0 | 0 | 0 | 0 | N | |
| F32341.B7b | Esophageal | 100 | 0 | 0 | 0 | 0 | 0 | N | |
| F00060962 | Esophageal | 98 | 0 | 2 | 0 | 2 | 4 | N | |
| F31283.C3b | Esophageal | 95 | 5 | 0 | 0 | 5 | 5 | N | |
| F4630.B6c | Esophageal | 95 | 5 | 0 | 0 | 5 | 5 | N | |
| F12661.B3a | Esophageal | 97 | 0 | 3 | 0 | 3 | 6 | N | Mixed pattern |
| F00060985 | Esophageal | 95 | 0 | 5 | 0 | 5 | 10 | N | Granular |
| F00060967 | Esophageal | 95 | 0 | 0 | 5 | 5 | 15 | N | |
| F19432.6c | Esophageal | 90 | 5 | 2 | 3 | 10 | 18 | N | Granular pattern |
| F00016274 | Esophageal | 90 | 10 | 2 | 3 | 15 | 23 | N | |
| F00060999 | Esophageal | 60 | 40 | 0 | 0 | 40 | 40 | N | |
| F00060992 | Esophageal | 75 | 0 | 25 | 0 | 25 | 50 | N | Granular |
| F00044229 | Esophageal | 80 | 0 | 0 | 20 | 20 | 60 | N | Positive normal tissue |
| F00061010 | Esophageal | 60 | 10 | 10 | 20 | 40 | 90 | N | |
| F31229.6c | Esophageal | 30 | 30 | 20 | 20 | 70 | 130 | N | |
| F32334.l6b | Esophageal | 10 | 0 | 20 | 70 | 90 | 250 | Y | Mixed pattern |
| F00060959 | Esophageal | 10 | 0 | 0 | 90 | 90 | 270 | N | |
| F31575.Fc | Esophageal | 0 | 0 | 30 | 70 | 100 | 270 | Y | |
| F00060972 | Esophageal | 0 | 0 | 0 | 100 | 100 | 300 | N | Granular |
| F31368.B8c | Esophageal | N/A | N/A | N/A | N/A | N/A | N/A | N/A | No definite tumor |
| 89-8184-1A | Gastric | 100 | 0 | 0 | 0 | 0 | 0 | N | Edge artifact |
| 92-1577-1H | Gastric | 100 | 0 | 0 | 0 | 0 | 0 | N | |
| 92-3118-B1 | Gastric | 100 | 0 | 0 | 0 | 0 | 0 | N | |
| F00042445 | Gastric | 100 | 0 | 0 | 0 | 0 | 0 | N | <1% |
| F00042483 | Gastric | 100 | 0 | 0 | 0 | 0 | 0 | N | |
| F00044305 | Gastric | 100 | 0 | 0 | 0 | 0 | 0 | N | |
| F00045485 | Gastric | 100 | 0 | 0 | 0 | 0 | 0 | N | <1% |
| 93-MC-2141- | Gastric | 99 | 1 | 0 | 0 | 1 | 1 | N | |
| B6 | | | | | | | | | |
| F00042438 | Gastric | 98 | 1 | 0 | 1 | 2 | 4 | N | Positive normal tissue |
| F12586.B4c | Gastric | 95 | 4 | 0 | 1 | 5 | 7 | N | |
| F11899.B3ba | Gastric | 97 | 2 | 0 | 2 | 4 | 8 | N | |
| F00064732 | Gastric | 95 | 0 | 5 | 0 | 5 | 10 | N | |
| 92-MC-4644- | Gastric | 80 | 20 | 0 | 0 | 20 | 20 | N | |
| B12 | | | | | | | | | |
| 3943-B2 | Gastric | 60 | 20 | 10 | 10 | 40 | 70 | N | |
| 91-MC-727-8 | Gastric | 75 | 0 | 0 | 25 | 25 | 75 | Y | |
| F00042004 | Gastric | 40 | 30 | 15 | 15 | 60 | 105 | Y | |
| 93-MC-1040- | Gastric | 50 | 15 | 10 | 25 | 50 | 110 | N | |
| 14 | | | | | | | | | |
| F00019678 | Gastric | 40 | 20 | 20 | 20 | 60 | 120 | N | |
| F00042331 | Gastric | 30 | 10 | 50 | 10 | 70 | 140 | N | |
| 94-MC-2834- | Gastric | 50 | 0 | 0 | 50 | 50 | 150 | N | |
| A6 | | | | | | | | | |
| F00042350 | Gastric | 25 | 0 | 0 | 75 | 75 | 225 | N | Mostly granular |
| F00019654 | Gastric | 0 | 0 | 50 | 50 | 100 | 250 | N | Mostly granular |
| | | | | | | | | | (positive normal tissue) |
| MC-6303-6 | Gastric | 0 | 20 | 10 | 70 | 100 | 250 | N | |
| F00042466 | Gastric | 0 | 0 | 40 | 60 | 100 | 260 | N | Mixed pattern |
| F00045456 | Gastric | 0 | 0 | 0 | 100 | 100 | 300 | N | |
| 89-MC-2391- | HCC | 100 | 0 | 0 | 0 | 0 | 0 | N | |
| B | | | | | | | | | |
| 89-MC-2595- | HCC | 100 | 0 | 0 | 0 | 0 | 0 | N | <1% |
| 2 | | | | | | | | | |
| 90-5184-1C | HCC | 100 | 0 | 0 | 0 | 0 | 0 | N | <1%. Endothelial cells |
| | | | | | | | | | positive |
| 92-148-6 | HCC | 100 | 0 | 0 | 0 | 0 | 0 | N | |
| 92-5221-1 | HCC | 100 | 0 | 0 | 0 | 0 | 0 | N | |
| 95-1159 | HCC | 100 | 0 | 0 | 0 | 0 | 0 | N | |
| 95-5405-B | HCC | 100 | 0 | 0 | 0 | 0 | 0 | N | <1%. Endothelial cells |
| | | | | | | | | | positive |
| 98-1952-A | HCC | 100 | 0 | 0 | 0 | 0 | 0 | N | |
| F00023224 | HCC | 100 | 0 | 0 | 0 | 0 | 0 | N | <1% |
| F00060528 | HCC | 100 | 0 | 0 | 0 | 0 | 0 | N | |
| F00067877 | HCC | 100 | 0 | 0 | 0 | 0 | 0 | N | <1% |
| F00069279 | HCC | 100 | 0 | 0 | 0 | 0 | 0 | N | <1% |
| F00083926 | HCC | 100 | 0 | 0 | 0 | 0 | 0 | N | |
| F25103.A1d | HCC | 100 | 0 | 0 | 0 | 0 | 0 | N | |
| F25133.Ab | HCC | 100 | 0 | 0 | 0 | 0 | 0 | N | |
| F25150.Ab | HCC | 100 | 0 | 0 | 0 | 0 | 0 | N | |
| F25231.A2d | HCC | 100 | 0 | 0 | 0 | 0 | 0 | N | <1% |
| F26606.0aa | HCC | 100 | 0 | 0 | 0 | 0 | 0 | N | <1% |
| F26618.Bba | HCC | 100 | 0 | 0 | 0 | 0 | 0 | N | |
| F00084251 | HCC | 95 | 3 | 0 | 2 | 5 | 9 | N | |
| F00083288 | HCC | 90 | 5 | 5 | 0 | 10 | 15 | N | |
| F00024144 | HCC | 90 | 0 | 0 | 10 | 10 | 30 | N | Granular |
| F25149.Ab | HCC | 90 | 0 | 0 | 10 | 10 | 30 | N | Granular |
| F25223.0b | HCC | N/A | N/A | N/A | N/A | N/A | N/A | N/A | No definite tumor |
| 00-13121-E8 | NSCLC | 100 | 0 | 0 | 0 | 0 | 0 | N | |
| 00-2193-C2 | NSCLC | 100 | 0 | 0 | 0 | 0 | 0 | N | |
| 98-1760-A6 | NSCLC | 100 | 0 | 0 | 0 | 0 | 0 | N | Positive in endothelial |
| | | | | | | | | | cells. |
| 98-4730-B2 | NSCLC | 100 | 0 | 0 | 0 | 0 | 0 | N | |
| 99-4637-E1 | NSCLC | 100 | 0 | 0 | 0 | 0 | 0 | N | <1% |
| 99-4847-E3 | NSCLC | 99 | 0 | 1 | 0 | 1 | 2 | N | |
| 00-11243-F3 | NSCLC | 99 | 0 | 0 | 1 | 1 | 3 | N | Mostly membranous |
| 99-4954-E3 | NSCLC | 99 | 0 | 0 | 1 | 1 | 3 | N | |
| 99-11059-A1 | NSCLC | 95 | 2 | 0 | 3 | 5 | 11 | N | |
| 00-3375-E2 | NSCLC | 95 | 0 | 0 | 5 | 5 | 15 | N | Mostly membranous |
| 99-11848-F6 | NSCLC | 95 | 0 | 0 | 5 | 5 | 15 | N | Mostly membranous |
| 00-13478-B5 | NSCLC | 90 | 5 | 0 | 5 | 10 | 20 | N | Mostly membranous |
| 96-1926-E7 | NSCLC | 90 | 5 | 0 | 5 | 10 | 20 | N | |
| 00-3057-A4 | NSCLC | 85 | 5 | 5 | 5 | 15 | 30 | N | |
| 99-8688-A2 | NSCLC | 75 | 15 | 5 | 5 | 25 | 40 | N | |
| 99-4893-B2 | NSCLC | 70 | 15 | 5 | 10 | 30 | 55 | N | |
| 89-6366-2F | NSCLC | 60 | 20 | 10 | 10 | 40 | 70 | N | Granular |
| 00-15401-F7 | NSCLC | 65 | 10 | 5 | 20 | 35 | 80 | N | |
| 89-3865-5C | NSCLC | 60 | 15 | 10 | 15 | 40 | 80 | N | |
| 99-4775-B3 | NSCLC | 50 | 30 | 10 | 10 | 50 | 80 | N | |
| 98-5744-C2 | NSCLC | 60 | 10 | 15 | 15 | 40 | 85 | N | |
| 00-14769-12 | NSCLC | 70 | 0 | 0 | 30 | 30 | 90 | N | |
| 00-8083-A3 | NSCLC | 50 | 25 | 10 | 15 | 50 | 90 | N | Mostly membranous |
| 00-9276-A1 | NSCLC | 70 | 0 | 0 | 30 | 30 | 90 | N | Granular |
| 00-3478-B6 | NSCLC | 50 | 10 | 10 | 30 | 50 | 120 | N | Mostly membranous |
| 99-11977-A2 | NSCLC | 40 | 20 | 10 | 30 | 60 | 130 | N | |
| 99-12031-B2 | NSCLC | 40 | 20 | 10 | 30 | 60 | 130 | N | Edge artifact |
| 98-17523-B2 | NSCLC | 25 | 0 | 0 | 75 | 75 | 225 | N | |
| 99-2656-A2 | NSCLC | 10 | 20 | 20 | 50 | 90 | 210 | N | Granular |
| 90-4304-4A | Pancreatic | 100 | 0 | 0 | 0 | 0 | 0 | N | |
| 97-346-F9 | Pancreatic | 100 | 0 | 0 | 0 | 0 | 0 | N | |
| 97-4775-F14 | Pancreatic | 100 | 0 | 0 | 0 | 0 | 0 | N | |
| 98-11790-C4 | Pancreatic | 100 | 0 | 0 | 0 | 0 | 0 | N | |
| 99-1671-C2 | Pancreatic | 100 | 0 | 0 | 0 | 0 | 0 | N | |
| F00021518 | Pancreatic | 100 | 0 | 0 | 0 | 0 | 0 | N | <1% |
| F00026443 | Pancreatic | 100 | 0 | 0 | 0 | 0 | 0 | N | <1% |
| F00026453 | Pancreatic | 100 | 0 | 0 | 0 | 0 | 0 | N | |
| 98-9163-D9 | Pancreatic | 99 | 0 | 0 | 1 | 1 | 3 | N | Granular |
| 92-7630-1 | Pancreatic | 95 | 5 | 0 | 0 | 5 | 5 | N | |
| F00026477 | Pancreatic | 95 | 5 | 0 | 0 | 5 | 5 | N | Positive pancreas |
| F00077923 | Pancreatic | 95 | 5 | 0 | 0 | 5 | 5 | N | |
| F00083302 | Pancreatic | 95 | 5 | 0 | 0 | 5 | 5 | N | Granular |
| 96-371-F13 | Pancreatic | 95 | 0 | 5 | 0 | 5 | 10 | N | Granular |
| F00026463 | Pancreatic | 90 | 5 | 0 | 5 | 10 | 20 | N | Positive normal tissue |
| F31023.2Ld | Pancreatic | 90 | 5 | 0 | 5 | 10 | 20 | N | Positive normal tissue |
| F00078326 | Pancreatic | 70 | 10 | 10 | 10 | 30 | 60 | N | |
| 97-10905-A6 | Pancreatic | 50 | 0 | 0 | 50 | 50 | 150 | N | Granular |
| F00080495 | Pancreatic | 10 | 20 | 20 | 50 | 90 | 210 | N | Granular |
| 94-4162-E10 | Pancreatic | 0 | 20 | 20 | 60 | 100 | 240 | N | |
| 93-8057-4M | Pancreatic | 10 | 0 | 0 | 90 | 90 | 270 | N | Granular |
| 93-8057-4O | Pancreatic | 0 | 0 | 10 | 90 | 100 | 290 | N | Granular |
| 94-7634-1 | Pancreatic | 0 | 0 | 0 | 100 | 100 | 300 | N | Granular |
| 98-5635-2E | Pancreatic | 0 | 0 | 0 | 100 | 100 | 300 | Y | Rare. More diffuse |
| | | | | | | | | | pattern. |
| 90-1771-3O | Pancreatic | N/A | N/A | N/A | N/A | N/A | N/A | N/A | Normal pancreas. |

APPENDIX 2
Globo-H IHC Results in CRC, Esophageal, Gastric, HCC, NSCLC, and Pancreatic Cancer TMAs

| | | | | | | | | | | Nuclear | |
|---|---|---|---|---|---|---|---|---|---|---|---|
| | | | | | | | | Total | | Staining | |
| | Core | | | Percentage of Tumor Cells | | | | Percent | H- | Observed? | |
| TMA ID | ID | Organ | Pathology | 0 | 1+ | 2+ | 3+ | Positive | score | Y/N | Comments |
| CO485 | A1 | Colon | Adenocarcinoma | 100 | 0 | 0 | 0 | 0 | 0 | N | |
| CO485 | A2 | Colon | Adenocarcinoma | 100 | 0 | 0 | 0 | 0 | 0 | N | |
| CO485 | A3 | Colon | Adenocarcinoma | 100 | 0 | 0 | 0 | 0 | 0 | N | |
| CO485 | A4 | Colon | Adenocarcinoma | 100 | 0 | 0 | 0 | 0 | 0 | N | |
| CO485 | A5 | Colon | Adenocarcinoma | 80 | 0 | 0 | 20 | 20 | 60 | Y | |
| | | | (chronic | | | | | | | | |
| | | | inflammation of | | | | | | | | |
| | | | mucosa) | | | | | | | | |
| CO485 | A6 | Colon | Adenocarcinoma | 100 | 0 | 0 | 0 | 0 | 0 | N | |
| CO485 | A7 | Colon | Adenocarcinoma | 100 | 0 | 0 | 0 | 0 | 0 | N | |
| CO485 | A8 | Colon | Adenocarcinoma | 99 | 1 | 0 | 0 | 1 | 1 | N | |
| CO485 | B1 | Colon | Adenocarcinoma | 90 | 10 | 0 | 0 | 10 | 10 | N | |
| CO485 | B2 | Colon | Adenocarcinoma | 95 | 0 | 5 | 0 | 5 | 10 | N | |
| CO485 | B3 | Colon | Adenocarcinoma | 100 | 0 | 0 | 0 | 0 | 0 | N | |
| CO485 | B4 | Colon | Adenocarcinoma | 100 | 0 | 0 | 0 | 0 | 0 | N | |
| CO485 | B5 | Colon | Adenocarcinoma | 100 | 0 | 0 | 0 | 0 | 0 | N | |
| | | | with necrosis | | | | | | | | |
| CO485 | B6 | Colon | Mucinous | 99 | 0 | 1 | 0 | 1 | 2 | N | |
| | | | adenocarcinoma | | | | | | | | |
| CO485 | B7 | Colon | Adenocarcinoma | 70 | 10 | 10 | 10 | 30 | 60 | Y | Occasional |
| CO485 | B8 | Colon | Adenocarcinoma | 100 | 0 | 0 | 0 | 0 | 0 | N | |
| CO485 | C1 | Colon | Adenocarcinoma | 100 | 0 | 0 | 0 | 0 | 0 | N | |
| CO485 | C2 | Colon | Adenocarcinoma | 95 | 0 | 5 | 0 | 5 | 10 | N | |
| CO485 | C3 | Colon | Adenocarcinoma | 100 | 0 | 0 | 0 | 0 | 0 | N | |
| CO485 | C4 | Colon | Adenocarcinoma | 95 | 0 | 0 | 5 | 5 | 15 | N | |
| CO485 | C5 | Colon | Adenocarcinoma | 100 | 0 | 0 | 0 | 0 | 0 | N | Rare |
| | | | | | | | | | | | tumor cells |
| | | | | | | | | | | | postive |
| | | | | | | | | | | | (<1%) |
| CO485 | C6 | Colon | Adenocarcinoma | 100 | 0 | 0 | 0 | 0 | 0 | N | |
| CO485 | C7 | Colon | Adenocarcinoma | 100 | 0 | 0 | 0 | 0 | 0 | N | |
| CO485 | C8 | Colon | Adenocarcinoma | 95 | 5 | 0 | 0 | 5 | 5 | N | |
| CO485 | D1 | Colon | Adenocarcinoma | 80 | 20 | 0 | 0 | 20 | 20 | N | |
| | | | (sparse) | | | | | | | | |
| CO485 | D2 | Colon | Adenocarcinoma | 60 | 30 | 10 | 0 | 40 | 50 | N | |
| CO485 | D3 | Colon | Adenocarcinoma | 100 | 0 | 0 | 0 | 0 | 0 | N | |
| CO485 | D4 | Colon | Adenocarcinoma | 100 | 0 | 0 | 0 | 0 | 0 | N | Rare |
| | | | | | | | | | | | tumor cells |
| | | | | | | | | | | | postive |
| | | | | | | | | | | | (<1%) |
| CO485 | D5 | Colon | Adenocarcinoma | 90 | 3 | 2 | 5 | 10 | 22 | N | |
| CO485 | D6 | Colon | Adenocarcinoma | 100 | 0 | 0 | 0 | 0 | 0 | N | |
| CO485 | D7 | Colon | Adenocarcinoma | 98 | 2 | 0 | 0 | 2 | 2 | N | |
| | | | (chronic | | | | | | | | |
| | | | inflammation of | | | | | | | | |
| | | | mucosa) | | | | | | | | |
| CO485 | D8 | Colon | Mucinous | 60 | 40 | 0 | 0 | 40 | 40 | N | |
| | | | adenocarcinoma | | | | | | | | |
| CO485 | E1 | Colon | Adenocarcinoma | N/A | N/A | N/A | N/A | N/A | N/A | N/A | Tissue |
| | | | | | | | | | | | missing |
| CO485 | E2 | Colon | Adenocarcinoma | 0 | 0 | 0 | 100 | 100 | 300 | Y | Occasional |
| CO485 | E3 | Colon | Adenocarcinoma | 30 | 20 | 20 | 30 | 70 | 150 | N | |
| CO485 | E4 | Colon | Adenocarcinoma | 100 | 0 | 0 | 0 | 0 | 0 | N | |
| CO485 | E5 | Colon | Adenocarcinoma | 95 | 3 | 0 | 2 | 5 | 9 | N | |
| CO485 | E6 | Colon | Adenocarcinoma | 100 | 0 | 0 | 0 | 0 | 0 | N | |
| CO485 | E7 | Colon | Adenocarcinoma | 100 | 0 | 0 | 0 | 0 | 0 | N | |
| CO485 | E8 | Colon | Adenocarcinoma | 0 | 0 | 0 | 100 | 100 | 300 | Y | Occasional |
| CO485 | F1 | Colon | Adenocarcinoma | 99 | 1 | 0 | 0 | 1 | 1 | N | |
| CO485 | F2 | Colon | Adenocarcinoma | 100 | 0 | 0 | 0 | 0 | 0 | N | |
| CO485 | F3 | Colon | Adenocarcinoma | 100 | 0 | 0 | 0 | 0 | 0 | N | |
| CO485 | F4 | Colon | Adenocarcinoma | N/A | N/A | N/A | N/A | N/A | N/A | N/A | Tissue |
| | | | | | | | | | | | missing |
| CO485 | F5 | Colon | Adenocarcinoma | 99 | 1 | 0 | 0 | 1 | 1 | N | |
| CO485 | F6 | Colon | Adenocarcinoma | 100 | 0 | 0 | 0 | 0 | 0 | N | |
| CO485 | F7 | Colon | Adenocarcinoma | 60 | 20 | 20 | 0 | 40 | 60 | N | |
| CO485 | F8 | Colon | Mucinous | 100 | 0 | 0 | 0 | 0 | 0 | N | |
| | | | adenocarcinoma | | | | | | | | |
| ES482 | A1 | Esophagus | Squamous cell | 70 | 30 | 0 | 0 | 30 | 30 | N | |
| | | | carcinoma | | | | | | | | |
| | | | (fibrous tissue | | | | | | | | |
| | | | and smooth | | | | | | | | |
| | | | muscle) | | | | | | | | |
| ES482 | A2 | Esophagus | Squamous cell | 75 | 10 | 5 | 10 | 25 | 50 | N | mostly |
| | | | carcinoma | | | | | | | | granular |
| | | | | | | | | | | | pattern |
| ES482 | A3 | Esophagus | Squamous cell | 100 | 0 | 0 | 0 | 0 | 0 | N | |
| | | | carcinoma | | | | | | | | |
| ES482 | A4 | Esophagus | Squamous cell | 50 | 50 | 0 | 0 | 50 | 50 | N | |
| | | | carcinoma | | | | | | | | |
| ES482 | A5 | Esophagus | Squamous cell | 80 | 5 | 5 | 10 | 20 | 45 | N | |
| | | | carcinoma | | | | | | | | |
| ES482 | A6 | Esophagus | Squamous cell | 75 | 10 | 5 | 10 | 25 | 50 | N | |
| | | | carcinoma | | | | | | | | |
| ES482 | A7 | Esophagus | Squamous cell | 100 | 0 | 0 | 0 | 0 | 0 | N | only few |
| | | | carcinoma | | | | | | | | tumor cells + |
| | | | | | | | | | | | (<1%) |
| ES482 | A8 | Esophagus | Squamous cell | 100 | 0 | 0 | 0 | 0 | 0 | N | |
| | | | carcinoma | | | | | | | | |
| | | | (fibrous tissue | | | | | | | | |
| | | | and smooth | | | | | | | | |
| | | | muscle) | | | | | | | | |
| ES482 | B1 | Esophagus | Squamous cell | 60 | 40 | 0 | 0 | 40 | 40 | N | |
| | | | carcinoma | | | | | | | | |
| ES482 | B2 | Esophagus | Squamous cell | 75 | 5 | 10 | 10 | 25 | 55 | N | |
| | | | carcinoma | | | | | | | | |
| ES482 | B3 | Esophagus | Squamous cell | 75 | 25 | 0 | 0 | 25 | 25 | N | |
| | | | carcinoma | | | | | | | | |
| ES482 | B4 | Esophagus | Squamous cell | 100 | 0 | 0 | 0 | 0 | 0 | N | only few |
| | | | carcinoma | | | | | | | | tumor cells + |
| | | | | | | | | | | | (<1%) |
| ES482 | B5 | Esophagus | Squamous cell | 100 | 0 | 0 | 0 | 0 | 0 | N | |
| | | | carcinoma | | | | | | | | |
| ES482 | B6 | Esophagus | Squamous cell | 40 | 20 | 20 | 20 | 60 | 120 | N | |
| | | | carcinoma | | | | | | | | |
| ES482 | B7 | Esophagus | Squamous cell | 60 | 20 | 20 | 0 | 40 | 60 | N | |
| | | | carcinoma | | | | | | | | |
| ES482 | B8 | Esophagus | Squamous cell | 75 | 15 | 10 | 0 | 25 | 35 | N | |
| | | | carcinoma | | | | | | | | |
| ES482 | C1 | Esophagus | Squamous cell | 25 | 25 | 25 | 25 | 75 | 150 | N | |
| | | | carcinoma | | | | | | | | |
| | | | (sparse) | | | | | | | | |
| ES482 | C2 | Esophagus | Squamous cell | 20 | 20 | 10 | 50 | 80 | 190 | N | |
| | | | carcinoma | | | | | | | | |
| ES482 | C3 | Esophagus | Carcinoma | 100 | 0 | 0 | 0 | 0 | 0 | N | |
| | | | tissue (sparse) | | | | | | | | |
| ES482 | C4 | Esophagus | Squamous cell | 75 | 15 | 10 | 0 | 25 | 35 | N | Granular |
| | | | carcinoma | | | | | | | | pattern |
| | | | (sparse) | | | | | | | | |
| ES482 | C5 | Esophagus | Squamous cell | 95 | 0 | 5 | 0 | 5 | 10 | N | |
| | | | carcinoma | | | | | | | | |
| ES482 | CE | Esophagus | Squamous cell | 100 | 0 | 0 | 0 | 0 | 0 | N | only few |
| | | | carcinoma | | | | | | | | tumor cells + |
| | | | | | | | | | | | (<1%) |
| ES482 | C7 | Esophagus | Squamous cell | 100 | 0 | 0 | 0 | 0 | 0 | N | only few |
| | | | carcinoma | | | | | | | | tumor cells + |
| | | | | | | | | | | | (<1%) |
| ES482 | C8 | Esophagus | Squamous cell | 80 | 10 | 10 | 0 | 20 | 30 | N | |
| | | | carcinoma | | | | | | | | |
| ES482 | D1 | Esophagus | Squamous cell | 30 | 10 | 10 | 50 | 70 | 180 | Y | |
| | | | carcinoma | | | | | | | | |
| ES482 | D2 | Esophagus | Squamous cell | 97 | 1 | 2 | 0 | 3 | 5 | N | |
| | | | carcinoma | | | | | | | | |
| ES482 | D3 | Esophagus | Squamous cell | 98 | 2 | 0 | 0 | 2 | 2 | N | |
| | | | carcinoma | | | | | | | | |
| ES482 | D4 | Esophagus | Squamous cell | 100 | 0 | 0 | 0 | 0 | 0 | N | |
| | | | carcinoma | | | | | | | | |
| ES482 | D5 | Esophagus | Squamous cell | 20 | 10 | 0 | 70 | 80 | 220 | Y | |
| | | | carcinoma | | | | | | | | |
| ES482 | D6 | Esophagus | Adenocarcinoma | 75 | 10 | 5 | 10 | 25 | 50 | Y | |
| ES482 | D7 | Esophagus | Adenocarcinoma | 100 | 0 | 0 | 0 | 0 | 0 | N | |
| ES482 | D8 | Esophagus | Adenocarcinoma | 60 | 20 | 20 | 0 | 40 | 60 | N | Granular |
| | | | | | | | | | | | pattern |
| ES482 | E1 | Esophagus | Adenocarcinoma | 60 | 20 | 20 | 0 | 40 | 60 | N | Granular |
| | | | | | | | | | | | pattern |
| ES482 | E2 | Esophagus | Adenocarcinoma | 100 | 0 | 0 | 0 | 0 | 0 | N | |
| ES482 | E3 | Esophagus | Adenocarcinoma | 100 | 0 | 0 | 0 | 0 | 0 | N | |
| ES482 | E4 | Esophagus | Adenocarcinoma | 97 | 3 | 0 | 0 | 3 | 3 | N | |
| ES482 | E5 | Esophagus | Adenocarcinoma | 100 | 0 | 0 | 0 | 0 | 0 | N | |
| ES482 | E6 | Esophagus | Adenocarcinoma | 100 | 0 | 0 | 0 | 0 | 0 | N | |
| ES482 | E7 | Esophagus | Small cell | 30 | 0 | 0 | 70 | 70 | 210 | N | Granular |
| | | | carcinoma | | | | | | | | pattern |
| ES482 | E8 | Esophagus | Carcinoma | 100 | 0 | 0 | 0 | 0 | 0 | N | |
| | | | sarcomatodes | | | | | | | | |
| ST484c | A1 | Stomach | Adenocarcinoma | 25 | 25 | 50 | 0 | 75 | 125 | N | |
| ST484c | A2 | Stomach | Adenocarcinoma | 100 | 0 | 0 | 0 | 0 | 0 | N | |
| ST484c | A3 | Stomach | Adenocarcinoma | 75 | 25 | 0 | 0 | 25 | 25 | N | |
| ST484c | A4 | Stomach | Adenocarcinoma | 95 | 5 | 0 | 0 | 5 | 5 | N | |
| ST484c | A5 | Stomach | Adenocarcinoma | 30 | 20 | 20 | 30 | 70 | 150 | Y | Occasional |
| ST484c | A6 | Stomach | Adenocarcinoma | 100 | 0 | 0 | 0 | 0 | 0 | N | |
| ST484c | A7 | Stomach | Adenocarcinoma | 99 | 0 | 1 | 0 | 1 | 2 | N | |
| ST484c | A8 | Stomach | Adenocarcinoma | 100 | 0 | 0 | 0 | 0 | 0 | N | |
| ST484c | B1 | Stomach | Adenocarcinoma | 100 | 0 | 0 | 0 | 0 | 0 | N | |
| ST484c | B2 | Stomach | Mucinous | 90 | 10 | 0 | 0 | 10 | 10 | N | |
| | | | adenocarcinoma | | | | | | | | |
| ST484c | B3 | Stomach | Adenocarcinoma | 80 | 10 | 0 | 10 | 20 | 40 | Y | Occasional |
| ST484c | B4 | Stomach | Adenocarcinoma | 100 | 0 | 0 | 0 | 0 | 0 | N | |
| ST484c | B5 | Stomach | Adenocarcinoma | 75 | 5 | 10 | 10 | 25 | 55 | N | |
| ST484c | B6 | Stomach | Adenocarcinoma | 100 | 0 | 0 | 0 | 0 | 0 | N | |
| ST484c | B7 | Stomach | Adenocarcinoma | 95 | 0 | 0 | 5 | 5 | 15 | N | |
| ST484c | B8 | Stomach | Adenocarcinoma | 70 | 0 | 10 | 20 | 30 | 80 | Y | Occasional |
| ST484c | C1 | Stomach | Adenocarcinoma | 100 | 0 | 0 | 0 | 0 | 0 | N | |
| ST484c | C2 | Stomach | Adenocarcinoma | 25 | 20 | 25 | 30 | 75 | 160 | Y | Occasional |
| ST484c | C3 | Stomach | Adenocarcinoma | 99 | 1 | 0 | 0 | 1 | 1 | N | |
| ST484c | C4 | Stomach | Adenocarcinoma | 100 | 0 | 0 | 0 | 0 | 0 | N | |
| ST484c | C5 | Stomach | Adenocarcinoma | 60 | 20 | 10 | 10 | 40 | 70 | Y | Occasional |
| ST484c | C6 | Stomach | Adenocarcinoma | 10 | 10 | 30 | 50 | 90 | 220 | Y | Occasional |
| ST484c | C7 | Stomach | Adenocarcinoma | 100 | 0 | 0 | 0 | 0 | 0 | N | |
| ST484c | C8 | Stomach | Adenocarcinoma | 0 | 0 | 0 | 100 | 100 | 300 | Y | Occasional |
| ST484c | D1 | Stomach | Lymph node | 100 | 0 | 0 | 0 | 0 | 0 | N | Rare |
| | | | metastasis of | | | | | | | | tumore |
| | | | adenocarcinoma | | | | | | | | cells |
| | | | | | | | | | | | positive |
| | | | | | | | | | | | (<1%) |
| ST484c | D2 | Stomach | Adenocarcinoma | 100 | 0 | 0 | 0 | 0 | 0 | N | |
| ST484c | D3 | Stomach | Adenocarcinoma | 100 | 0 | 0 | 0 | 0 | 0 | N | |
| ST484c | D4 | Stomach | Adenocarcinoma | 98 | 2 | 0 | 0 | 2 | 2 | N | |
| ST484c | D5 | Stomach | Adenocarcinoma | 99 | 0 | 1 | 0 | 1 | 2 | N | |
| ST484c | D6 | Stomach | Adenocarcinoma | 95 | 0 | 5 | 0 | 5 | 10 | N | |
| ST484c | D7 | Stomach | Adenocarcinoma | 95 | 0 | 0 | 5 | 5 | 15 | Y | Occasional |
| ST484c | D8 | Stomach | Adenocarcinoma | 100 | 0 | 0 | 0 | 0 | 0 | N | |
| ST484c | E1 | Stomach | Adenocarcinoma | 100 | 0 | 0 | 0 | 0 | 0 | N | |
| ST484c | E2 | Stomach | Adenocarcinoma | 100 | 0 | 0 | 0 | 0 | 0 | N | |
| ST484c | E3 | Stomach | Adenocarcinoma | 98 | 2 | 0 | 0 | 2 | 2 | N | |
| ST484c | E4 | Stomach | Mucinous | 100 | 0 | 0 | 0 | 0 | 0 | N | |
| | | | adenocarcinoma | | | | | | | | |
| ST484c | E5 | Stomach | Adenocarcinoma | 100 | 0 | 0 | 0 | 0 | 0 | N | |
| ST484c | E6 | Stomach | Adenocarcinoma | 60 | 0 | 20 | 20 | 40 | 100 | N | |
| ST484c | E7 | Stomach | Adenocarcinoma | 10 | 0 | 0 | 90 | 90 | 270 | Y | |
| ST484c | E8 | Stomach | Adenocarcinoma | 100 | 0 | 0 | 0 | 0 | 0 | N | |
| ST484c | F1 | Stomach | Adenocarcinoma | 60 | 10 | 20 | 10 | 40 | 80 | Y | |
| ST484c | F2 | Stomach | Adenocarcinoma | 10 | 0 | 0 | 90 | 90 | 270 | Y | |
| ST484c | F3 | Stomach | Adenocarcinoma | 100 | 0 | 0 | 0 | 0 | 0 | N | |
| ST484c | F4 | Stomach | Adenocarcinoma | 70 | 10 | 10 | 10 | 30 | 60 | Y | Occasional |
| ST484c | F5 | Stomach | Adenocarcinoma | 100 | 0 | 0 | 0 | 0 | 0 | N | |
| ST484c | F6 | Stomach | Adenocarcinoma | 100 | 0 | 0 | 0 | 0 | 0 | N | |
| ST484c | F7 | Stomach | Mucinous | 100 | 0 | 0 | 0 | 0 | 0 | N | |
| | | | adenocarcinoma | | | | | | | | |
| ST484c | F8 | Stomach | Adenocarcinoma | 100 | 0 | 0 | 0 | 0 | 0 | N | |
| LV484 | A1 | Liver | Hepatocellular | 100 | 0 | 0 | 0 | 0 | 0 | N | |
| | | | carcinoma | | | | | | | | |
| LV484 | A2 | Liver | Hepatocellular | 100 | 0 | 0 | 0 | 0 | 0 | N | |
| | | | carcinoma | | | | | | | | |
| LV484 | A3 | Liver | Hepatocellular | 99 | 1 | 0 | 0 | 1 | 1 | N | |
| | | | carcinoma | | | | | | | | |
| LV484 | A4 | Liver | Hepatocellular | 100 | 0 | 0 | 0 | 0 | 0 | N | Rare |
| | | | carcinoma | | | | | | | | Tumor |
| | | | | | | | | | | | cells |
| | | | | | | | | | | | positive |
| | | | | | | | | | | | (<1%) |
| LV484 | A5 | Liver | Hepatocellular | 100 | 0 | 0 | 0 | 0 | 0 | N | Scant |
| | | | carcinoma | | | | | | | | tissue |
| LV484 | A6 | Liver | Hepatocellular | 100 | 0 | 0 | 0 | 0 | 0 | N | |
| | | | carcinoma | | | | | | | | |
| LV484 | A7 | Liver | Hepatocellular | 100 | 0 | 0 | 0 | 0 | 0 | N | |
| | | | carcinoma | | | | | | | | |
| LV484 | A8 | Liver | Hepatocellular | N/A | N/A | N/A | N/A | N/A | N/A | N/A | Stain faded |
| | | | carcinoma | | | | | | | | |
| LV484 | B1 | Liver | Hepatocellular | 100 | 0 | 0 | 0 | 0 | 0 | N | |
| | | | carcinoma | | | | | | | | |
| LV484 | B2 | Liver | Hepatocellular | 100 | 0 | 0 | 0 | 0 | 0 | N | |
| | | | carcinoma | | | | | | | | |
| LV484 | B3 | Liver | Hepatocellular | 95 | 5 | 0 | 0 | 5 | 5 | N | |
| | | | carcinoma | | | | | | | | |
| LV484 | B4 | Liver | Hepatocellular | 95 | 5 | 0 | 0 | 5 | 5 | N | |
| | | | carcinoma | | | | | | | | |
| LV484 | B5 | Liver | Hepatocellular | 100 | 0 | 0 | 0 | 0 | 0 | N | Rare |
| | | | carcinoma | | | | | | | | Tumor |
| | | | | | | | | | | | cells |
| | | | | | | | | | | | positive |
| | | | | | | | | | | | (<1%) |
| LV484 | B6 | Liver | Hepatocellular | 80 | 15 | 5 | 0 | 20 | 25 | N | |
| | | | carcinoma | | | | | | | | |
| LV484 | B7 | Liver | Hepatocellular | 100 | 0 | 0 | 0 | 0 | 0 | N | |
| | | | carcinoma | | | | | | | | |
| LV484 | B8 | Liver | Hepatocellular | 100 | 0 | 0 | 0 | 0 | 0 | N | |
| | | | carcinoma | | | | | | | | |
| LV484 | C1 | Liver | Hepatocellular | 100 | 0 | 0 | 0 | 0 | 0 | N | |
| | | | carcinoma | | | | | | | | |
| LV484 | C2 | Liver | Hepatocellular | 100 | 0 | 0 | 0 | 0 | 0 | N | |
| | | | carcinoma | | | | | | | | |
| LV484 | C3 | Liver | Hepatocellular | 100 | 0 | 0 | 0 | 0 | 0 | N | |
| | | | carcinoma | | | | | | | | |
| LV484 | C4 | Liver | Hepatocellular | 95 | 5 | 0 | 0 | 5 | 5 | N | |
| | | | carcinoma | | | | | | | | |
| LV484 | C5 | Liver | Hepatocellular | 100 | 0 | 0 | 0 | 0 | 0 | N | |
| | | | carcinoma | | | | | | | | |
| LV484 | C6 | Liver | Hepatocellular | 100 | 0 | 0 | 0 | 0 | 0 | N | |
| | | | carcinoma | | | | | | | | |
| LV484 | C7 | Liver | Hepatocellular | 100 | 0 | 0 | 0 | 0 | 0 | N | |
| | | | carcinoma | | | | | | | | |
| LV484 | C8 | Liver | Hepatocellular | 100 | 0 | 0 | 0 | 0 | 0 | N | |
| | | | carcinoma | | | | | | | | |
| LV484 | D1 | Liver | Hepatocellular | 100 | 0 | 0 | 0 | 0 | 0 | N | |
| | | | carcinoma | | | | | | | | |
| LV484 | D2 | Liver | Hepatocellular | 100 | 0 | 0 | 0 | 0 | 0 | N | Rare |
| | | | carcinoma | | | | | | | | Tumor |
| | | | | | | | | | | | cells |
| | | | | | | | | | | | positive |
| | | | | | | | | | | | (<1%) |
| LV484 | D3 | Liver | Hepatocellular | 100 | 0 | 0 | 0 | 0 | 0 | N | |
| | | | carcinoma | | | | | | | | |
| LV484 | D4 | Liver | Hepatocellular | 100 | 0 | 0 | 0 | 0 | 0 | N | |
| | | | carcinoma | | | | | | | | |
| LV484 | D5 | Liver | Hepatocellular | 100 | 0 | 0 | 0 | 0 | 0 | N | |
| | | | carcinoma | | | | | | | | |
| LV484 | D6 | Liver | Hepatocellular | 100 | 0 | 0 | 0 | 0 | 0 | N | |
| | | | carcinoma | | | | | | | | |
| LV484 | D7 | Liver | Hepatocellular | 100 | 0 | 0 | 0 | 0 | 0 | N | |
| | | | carcinoma | | | | | | | | |
| LV484 | D8 | Liver | Hepatocellular | 100 | 0 | 0 | 0 | 0 | 0 | N | |
| | | | carcinoma | | | | | | | | |
| LV484 | E1 | Liver | Hepatocellular | 100 | 0 | 0 | 0 | 0 | 0 | N | Rare |
| | | | carcinoma | | | | | | | | Tumor |
| | | | | | | | | | | | cells |
| | | | | | | | | | | | positive |
| | | | | | | | | | | | (<1%) |
| LV484 | E2 | Liver | Hepatocellular | 99 | 1 | 0 | 0 | 1 | 1 | N | |
| | | | carcinoma | | | | | | | | |
| LV484 | E3 | Liver | Hepatocellular | 100 | 0 | 0 | 0 | 0 | 0 | N | |
| | | | carcinoma | | | | | | | | |
| LV484 | E4 | Liver | Hepatocellular | 100 | 0 | 0 | 0 | 0 | 0 | N | |
| | | | carcinoma | | | | | | | | |
| LV484 | E5 | Liver | Hepatocellular | 100 | 0 | 0 | 0 | 0 | 0 | N | |
| | | | carcinoma | | | | | | | | |
| LV484 | E6 | Liver | Hepatocellular | 100 | 0 | 0 | 0 | 0 | 0 | N | |
| | | | carcinoma with | | | | | | | | |
| | | | necrosis | | | | | | | | |
| LV484 | E7 | Liver | Hepatocellular | 95 | 5 | 0 | 0 | 5 | 5 | N | |
| | | | carcinoma | | | | | | | | |
| LV484 | E8 | Liver | Hepatocellular | 100 | 0 | 0 | 0 | 0 | 0 | N | |
| | | | carcinoma | | | | | | | | |
| | | | (tumoral | | | | | | | | |
| | | | necrosis) | | | | | | | | |
| LV484 | F1 | Liver | Cholangiocellular | 70 | 0 | 10 | 20 | 30 | 80 | N | |
| | | | carcinoma | | | | | | | | |
| LV484 | F2 | Liver | Cholangiocellular | 100 | 0 | 0 | 0 | 0 | 0 | N | |
| | | | carcinoma with | | | | | | | | |
| | | | necrosis | | | | | | | | |
| LV484 | F3 | Liver | Cholangiocellular | 100 | 0 | 0 | 0 | 0 | 0 | N | |
| | | | carcinoma | | | | | | | | |
| LV484 | F4 | Liver | Cholangiocellular | 100 | 0 | 0 | 0 | 0 | 0 | N | |
| | | | carcinoma | | | | | | | | |
| LV484 | F5 | Liver | Cholangiocellular | 100 | 0 | 0 | 0 | 0 | 0 | N | |
| | | | carcinoma with | | | | | | | | |
| | | | necrosis | | | | | | | | |
| LV484 | F6 | Liver | Cholangiocellular | 100 | 0 | 0 | 0 | 0 | 0 | N | |
| | | | carcinoma | | | | | | | | |
| LV484 | F7 | Liver | Cholangiocellular | 100 | 0 | 0 | 0 | 0 | 0 | N | |
| | | | carcinoma | | | | | | | | |
| LV484 | F8 | Liver | Cholangiocellular | 100 | 0 | 0 | 0 | 0 | 0 | N | Rare |
| | | | carcinoma | | | | | | | | Tumor |
| | | | | | | | | | | | cells |
| | | | | | | | | | | | positive |
| | | | | | | | | | | | (<1%) |
| LC484 | A1 | Lung | Squamous cell | 100 | 0 | 0 | 0 | 0 | 0 | N | Only |
| | | | carcinoma | | | | | | | | necrotic |
| | | | | | | | | | | | tissue or Ic |
| | | | | | | | | | | | stained |
| | | | | | | | | | | | positive |
| LC484 | A2 | Lung | Squamous cell | 100 | 0 | 0 | 0 | 0 | 0 | N | |
| | | | carcinoma | | | | | | | | |
| LC484 | A3 | Lung | Squamous cell | 99 | 1 | 0 | 0 | 1 | 1 | N | |
| | | | carcinoma | | | | | | | | |
| LC484 | A4 | Lung | Squamous cell | 100 | 0 | 0 | 0 | 0 | 0 | N | |
| | | | carcinoma | | | | | | | | |
| LC484 | A5 | Lung | Squamous cell | 100 | 0 | 0 | 0 | 0 | 0 | N | Rare |
| | | | carcinoma | | | | | | | | tumor cells |
| | | | | | | | | | | | positive |
| | | | | | | | | | | | (<1%) |
| LC484 | A6 | Lung | Squamous cell | 90 | 10 | 0 | 0 | 10 | 10 | N | |
| | | | carcinoma | | | | | | | | |
| LC484 | A7 | Lung | Squamous cell | 98 | 1 | 0 | 1 | 2 | 4 | N | |
| | | | carcinoma | | | | | | | | |
| LC484 | A8 | Lung | Squamous cell | 100 | 0 | 0 | 0 | 0 | 0 | N | |
| | | | carcinoma | | | | | | | | |
| LC484 | B1 | Lung | Squamous cell | 100 | 0 | 0 | 0 | 0 | 0 | N | |
| | | | carcinoma | | | | | | | | |
| LC484 | B2 | Lung | Squamous cell | 100 | 0 | 0 | 0 | 0 | 0 | N | |
| | | | carcinoma | | | | | | | | |
| | | | (chronic | | | | | | | | |
| | | | inflammation) | | | | | | | | |
| LC484 | B3 | Lung | Squamous cell | 100 | 0 | 0 | 0 | 0 | 0 | N | |
| | | | carcinoma | | | | | | | | |
| LC484 | B4 | Lung | Squamous cell | 100 | 0 | 0 | 0 | 0 | 0 | N | |
| | | | carcinoma | | | | | | | | |
| LC484 | B5 | Lung | Squamous cell | 100 | 0 | 0 | 0 | 0 | 0 | N | Occasional |
| | | | carcinoma | | | | | | | | endothelial |
| | | | | | | | | | | | cells are |
| | | | | | | | | | | | positive |
| LC484 | B6 | Lung | Squamous cell | 97 | 3 | 0 | 0 | 3 | 3 | N | Normal |
| | | | carcinoma | | | | | | | | alveolar |
| | | | | | | | | | | | epithelial |
| | | | | | | | | | | | cell postive |
| LC484 | B7 | Lung | Squamous cell | 100 | 0 | 0 | 0 | 0 | 0 | N | |
| | | | carcinoma | | | | | | | | |
| LC484 | B8 | Lung | Squamous cell | 100 | 0 | 0 | 0 | 0 | 0 | N | |
| | | | carcinoma | | | | | | | | |
| LC484 | C1 | Lung | Squamous cell | 50 | 50 | 0 | 0 | 50 | 50 | N | |
| | | | carcinoma | | | | | | | | |
| LC484 | C2 | Lung | Squamous cell | 100 | 0 | 0 | 0 | 0 | 0 | N | |
| | | | carcinoma | | | | | | | | |
| LC484 | C3 | Lung | Squamous cell | 100 | 0 | 0 | 0 | 0 | 0 | N | |
| | | | carcinoma | | | | | | | | |
| LC484 | C4 | Lung | Squamous cell | 100 | 0 | 0 | 0 | 0 | 0 | N | Occasional |
| | | | carcinoma | | | | | | | | endothelial |
| | | | | | | | | | | | cells are |
| | | | | | | | | | | | positive |
| LC484 | C5 | Lung | Squamous cell | 95 | 2 | 0 | 3 | 5 | 11 | N | |
| | | | carcinoma | | | | | | | | |
| LC484 | C6 | Lung | Squamous cell | 100 | 0 | 0 | 0 | 0 | 0 | N | |
| | | | carcinoma | | | | | | | | |
| LC484 | C7 | Lung | Squamous cell | 95 | 0 | 0 | 5 | 5 | 15 | N | |
| | | | carcinoma | | | | | | | | |
| LC484 | C8 | Lung | Squamous cell | 100 | 0 | 0 | 0 | 0 | 0 | N | |
| | | | carcinoma | | | | | | | | |
| LC484 | D1 | Lung | Squamous cell | 95 | 5 | 0 | 0 | 5 | 5 | N | |
| | | | carcinoma | | | | | | | | |
| LC484 | D2 | Lung | Squamous cell | 99 | 1 | 0 | 0 | 1 | 1 | N | |
| | | | carcinoma | | | | | | | | |
| LC484 | D3 | Lung | Squamous cell | 20 | 30 | 50 | 0 | 80 | 130 | N | |
| | | | carcinoma | | | | | | | | |
| LC484 | D4 | Lung | Squamous cell | 20 | 40 | 40 | 0 | 80 | 120 | N | |
| | | | carcinoma | | | | | | | | |
| LC484 | D5 | Lung | Squamous cell | 95 | 5 | 0 | 0 | 5 | 5 | N | |
| | | | carcinoma | | | | | | | | |
| LC484 | D6 | Lung | Squamous cell | 100 | 0 | 0 | 0 | 0 | 0 | N | |
| | | | carcinoma | | | | | | | | |
| LC484 | D7 | Lung | Squamous cell | 95 | 2 | 3 | 0 | 5 | 8 | N | |
| | | | carcinoma | | | | | | | | |
| LC484 | D8 | Lung | Squamous cell | 100 | 0 | 0 | 0 | 0 | 0 | N | |
| | | | carcinoma | | | | | | | | |
| LC484 | E1 | Lung | Squamous cell | 99 | 1 | 0 | 0 | 1 | 1 | N | stromal |
| | | | carcinoma | | | | | | | | cells |
| | | | | | | | | | | | positive |
| LC484 | E2 | Lung | Squamous cell | 40 | 20 | 20 | 20 | 60 | 120 | N | |
| | | | carcinoma | | | | | | | | |
| LC484 | E3 | Lung | Squamous cell | 100 | 0 | 0 | 0 | 0 | 0 | N | |
| | | | carcinoma | | | | | | | | |
| LC484 | E4 | Lung | Squamous cell | 90 | 5 | 5 | 0 | 10 | 15 | N | |
| | | | carcinoma | | | | | | | | |
| LC484 | E5 | Lung | Adenosquamous | 100 | 0 | 0 | 0 | 0 | 0 | N | |
| | | | carcinoma | | | | | | | | |
| LC484 | E6 | Lung | Adenocarcinoma | 100 | 0 | 0 | 0 | 0 | 0 | N | |
| LC484 | E7 | Lung | Adenocarcinoma | 80 | 20 | 0 | 0 | 20 | 20 | N | |
| LC484 | E8 | Lung | Adenocarcinoma | 100 | 0 | 0 | 0 | 0 | 0 | N | |
| LC484 | F1 | Lung | Adenocarcinoma | 100 | 0 | 0 | 0 | 0 | 0 | N | |
| LC484 | F2 | Lung | Adenocarcinoma | 95 | 0 | 5 | 0 | 5 | 10 | N | |
| LC484 | F3 | Lung | Undifferentiated | 100 | 0 | 0 | 0 | 0 | 0 | N | |
| | | | carcinoma | | | | | | | | |
| LC484 | F4 | Lung | Small cell | 97 | 3 | 0 | 0 | 3 | 3 | N | |
| | | | undifferentiated | | | | | | | | |
| | | | carcinoma | | | | | | | | |
| LC484 | F5 | Lung | Small cell | 60 | 20 | 20 | 0 | 40 | 60 | N | Scant |
| | | | undifferentiated | | | | | | | | tumor |
| | | | carcinoma | | | | | | | | |
| | | | (sparse) | | | | | | | | |
| LC484 | F6 | Lung | Small cell | 100 | 0 | 0 | 0 | 0 | 0 | N | |
| | | | undifferentiated | | | | | | | | |
| | | | carcinoma | | | | | | | | |
| LC484 | F7 | Lung | Small cell | 95 | 5 | 0 | 0 | 5 | 5 | N | |
| | | | undifferentiated | | | | | | | | |
| | | | carcinoma | | | | | | | | |
| LC484 | F8 | Lung | Large cell | 100 | 0 | 0 | 0 | 0 | 0 | N | |
| | | | carcinoma | | | | | | | | |
| AGTA- | A1 | Pancreas | Pancreatic | 100 | 0 | 0 | 0 | 0 | 0 | N | |
| 90 | | | cancer | | | | | | | | |
| AGTA- | A2 | Pancreas | Pancreatic | 100 | 0 | 0 | 0 | 0 | 0 | N | |
| 90 | | | cancer | | | | | | | | |
| AGTA- | A3 | Pancreas | Pancreatic | 100 | 0 | 0 | 0 | 0 | 0 | N | |
| 90 | | | cancer | | | | | | | | |
| AGTA- | A4 | Pancreas | Pancreatic | N/A | N/A | N/A | N/A | N/A | N/A | N/A | Tissue |
| 90 | | | cancer | | | | | | | | missing |
| AGTA- | A5 | Pancreas | Pancreatic | 70 | 20 | 0 | 10 | 30 | 50 | N | |
| 90 | | | cancer | | | | | | | | |
| AGTA- | A6 | Pancreas | Pancreatic | 30 | 30 | 0 | 40 | 70 | 150 | y | |
| 90 | | | cancer | | | | | | | | |
| AGTA- | A7 | Pancreas | Pancreatic | 100 | 0 | 0 | 0 | 0 | 0 | N | |
| 90 | | | cancer | | | | | | | | |
| AGTA- | A8 | Pancreas | Pancreatic | 100 | 0 | 0 | 0 | 0 | 0 | N | Normal |
| 90 | | | cancer | | | | | | | | tissue |
| | | | | | | | | | | | stained |
| AGTA- | A9 | Pancreas | Pancreatic | 100 | 0 | 0 | 0 | 0 | 0 | N | |
| 90 | | | cancer | | | | | | | | |
| AGTA- | A10 | Pancreas | Pancreatic | 0 | 0 | 0 | 100 | 100 | 300 | N | |
| 90 | | | cancer | | | | | | | | |
| AGTA- | A11 | Pancreas | Pancreatic | 75 | 0 | 0 | 25 | 25 | 75 | N | Granular |
| 90 | | | cancer | | | | | | | | pattern |
| AGTA- | A12 | Blank | Pancreatic | N/A | N/A | N/A | N/A | N/A | N/A | N/A | No Core |
| 90 | | | cancer | | | | | | | | |
| AGTA- | A13 | Pancreas | Pancreatic | N/A | N/A | N/A | N/A | N/A | N/A | N/A | unreadable |
| 90 | | | cancer | | | | | | | | tissue |
| | | | | | | | | | | | (rolled |
| | | | | | | | | | | | over) |
| AGTA- | A14 | Pancreas | Pancreatic | 100 | 0 | 0 | 0 | 0 | 0 | N | |
| 90 | | | cancer | | | | | | | | |
| AGTA- | A15 | Pancreas | Pancreatic | N/A | N/A | N/A | N/A | N/A | N/A | N/A | No defnite |
| 90 | | | cancer | | | | | | | | tumor |
| | | | | | | | | | | | seen |
| | | | | | | | | | | | (normal |
| | | | | | | | | | | | tissue |
| | | | | | | | | | | | stained) |
| AGTA- | A16 | Pancreas | Pancreatic | 75 | 25 | 0 | 0 | 25 | 25 | N | normal |
| 90 | | | cancer | | | | | | | | tissue |
| | | | | | | | | | | | stained |
| AGTA- | A17 | Pancreas | Pancreatic | N/A | N/A | N/A | N/A | N/A | N/A | N/A | No defnite |
| 90 | | | cancer | | | | | | | | tumor |
| | | | | | | | | | | | seen |
| | | | | | | | | | | | (normal |
| | | | | | | | | | | | tissue |
| | | | | | | | | | | | stained) |
| AGTA- | A18 | Pancreas | Pancreatic | N/A | N/A | N/A | N/A | N/A | N/A | N/A | unreadable |
| 90 | | | cancer | | | | | | | | tissue |
| | | | | | | | | | | | (rolled |
| | | | | | | | | | | | over) |
| AGTA- | B1 | Pancreas | Pancreatic | 100 | 25 | 0 | 75 | 100 | 250 | Y | |
| 90 | | | cancer | | | | | | | | |
| AGTA- | B2 | Pancreas | Pancreatic | 0 | 0 | 0 | 100 | 100 | 300 | Y | |
| 90 | | | cancer | | | | | | | | |
| AGTA- | B3 | Pancreas | Pancreatic | 0 | 0 | 0 | 100 | 100 | 300 | Y | |
| 90 | | | cancer | | | | | | | | |
| AGTA- | B4 | Pancreas | Pancreatic | 0 | 25 | 25 | 50 | 100 | 225 | Y | |
| 90 | | | cancer | | | | | | | | |
| AGTA- | B5 | Pancreas | Pancreatic | 0 | 0 | 0 | 100 | 100 | 300 | Y | |
| 90 | | | cancer | | | | | | | | |
| AGTA- | B6 | Pancreas | Pancreatic | 0 | 0 | 0 | 100 | 100 | 300 | N | Granular |
| 90 | | | cancer | | | | | | | | pattern |
| AGTA- | B7 | Pancreas | Pancreatic | 50 | 10 | 20 | 20 | 50 | 110 | N | Granular |
| 90 | | | cancer | | | | | | | | pattern |
| AGTA- | B8 | Pancreas | Pancreatic | 25 | 30 | 25 | 20 | 75 | 140 | N | Granular |
| 90 | | | cancer | | | | | | | | pattern |
| AGTA- | B9 | Pancreas | Pancreatic | 0 | 0 | 0 | 100 | 100 | 300 | N | |
| 90 | | | cancer | | | | | | | | |
| AGTA- | B10 | Pancreas | Pancreatic | 0 | 0 | 0 | 100 | 100 | 300 | Y | |
| 90 | | | cancer | | | | | | | | |
| AGTA- | B11 | Pancreas | Pancreatic | N/A | N/A | N/A | N/A | N/A | N/A | N/A | No tumor |
| 90 | | | cancer | | | | | | | | seen, |
| | | | | | | | | | | | Normal |
| | | | | | | | | | | | tissue |
| | | | | | | | | | | | stained |
| AGTA- | B12 | Pancreas | Pancreatic | 0 | 25 | 0 | 75 | 100 | 250 | N | Normal |
| 90 | | | cancer | | | | | | | | tissue |
| | | | | | | | | | | | stained |
| AGTA- | B13 | Pancreas | Pancreatic | N/A | N/A | N/A | N/A | N/A | N/A | N/A | No tumor |
| 90 | | | cancer | | | | | | | | seen, |
| | | | | | | | | | | | Normal |
| | | | | | | | | | | | tissue |
| | | | | | | | | | | | stained |
| AGTA- | B14 | Pancreas | Pancreatic | N/A | N/A | N/A | N/A | N/A | N/A | N/A | No tumor |
| 90 | | | cancer | | | | | | | | seen, |
| | | | | | | | | | | | Normal |
| | | | | | | | | | | | tissue |
| | | | | | | | | | | | stained |
| AGTA- | B15 | Pancreas | Pancreatic | 0 | 0 | 100 | 0 | 100 | 200 | N | Granular |
| 90 | | | cancer | | | | | | | | pattern |
| AGTA- | B16 | Pancreas | Pancreatic | 0 | 0 | 50 | 50 | 100 | 250 | N | Granular |
| 90 | | | cancer | | | | | | | | pattern |
| AGTA- | B17 | Pancreas | Pancreatic | 25 | 20 | 20 | 35 | 75 | 165 | N | Granular |
| 90 | | | cancer | | | | | | | | pattern |
| AGTA- | B18 | Blank | Pancreatic | N/A | N/A | N/A | N/A | N/A | N/A | N/A | No Core |
| 90 | | | cancer | | | | | | | | |
| AGTA- | C1 | Pancreas | Pancreatic | 0 | 0 | 0 | 100 | 100 | 300 | Y | |
| 90 | | | cancer | | | | | | | | |
| AGTA- | C2 | Pancreas | Pancreatic | 50 | 0 | 50 | 0 | 50 | 100 | N | Granular |
| 90 | | | cancer | | | | | | | | pattern |
| AGTA- | C3 | Pancreas | Pancreatic | N/A | N/A | N/A | N/A | N/A | N/A | N/A | Tissue fell |
| 90 | | | cancer | | | | | | | | off |
| AGTA- | C4 | Pancreas | Pancreatic | N/A | N/A | N/A | N/A | N/A | N/A | N/A | Tissue |
| 90 | | | cancer | | | | | | | | rolled over |
| AGTA- | C5 | Pancreas | Pancreatic | 30 | 20 | 20 | 30 | 70 | 150 | N | |
| 90 | | | cancer | | | | | | | | |
| AGTA- | C6 | Pancreas | Pancreatic | 30 | 20 | 20 | 30 | 70 | 150 | N | |
| 90 | | | cancer | | | | | | | | |
| AGTA- | C7 | Pancreas | Pancreatic | 0 | 50 | 50 | 0 | 100 | 150 | N | Granular |
| 90 | | | cancer | | | | | | | | pattern |
| AGTA- | C8 | Pancreas | Pancreatic | N/A | N/A | N/A | N/A | N/A | N/A | N/A | No defnite |
| 90 | | | cancer | | | | | | | | tumor |
| | | | | | | | | | | | identified |
| AGTA- | C9 | Pancreas | Pancreatic | N/A | N/A | N/A | N/A | N/A | N/A | N/A | No defnite |
| 90 | | | cancer | | | | | | | | tumor |
| | | | | | | | | | | | identified |
| AGTA- | C10 | Pancreas | Pancreatic | N/A | N/A | N/A | N/A | N/A | N/A | N/A | No defnite |
| 90 | | | cancer | | | | | | | | tumor |
| | | | | | | | | | | | identified |
| AGTA- | C11 | Blank | Pancreatic | N/A | N/A | N/A | N/A | N/A | N/A | N/A | No Core |
| 90 | | | cancer | | | | | | | | |
| AGTA- | C12 | Pancreas | Pancreatic | 75 | 10 | 10 | 5 | 25 | 45 | N | |
| 90 | | | cancer | | | | | | | | |
| AGTA- | C13 | Pancreas | Pancreatic | N/A | N/A | N/A | N/A | N/A | N/A | N/A | Unreadable |
| 90 | | | cancer | | | | | | | | tissue, ?? |
| | | | | | | | | | | | Edge |
| | | | | | | | | | | | artifact |
| AGTA- | C14 | Pancreas | Pancreatic | 0 | 0 | 0 | 100 | 100 | 300 | N | Granular |
| 90 | | | cancer | | | | | | | | pattern |
| AGTA- | C15 | Pancreas | Pancreatic | 60 | 20 | 20 | 0 | 40 | 60 | N | Granular |
| 90 | | | cancer | | | | | | | | pattern |
| AGTA- | C16 | Pancreas | Pancreatic | 75 | 0 | 0 | 25 | 25 | 75 | Y | |
| 90 | | | cancer | | | | | | | | |
| AGTA- | C17 | Pancreas | Pancreatic | 100 | 0 | 0 | 0 | 0 | 0 | N | |
| 90 | | | cancer | | | | | | | | |
| AGTA- | C18 | Pancreas | Pancreatic | 40 | 20 | 20 | 20 | 60 | 120 | N | |
| 90 | | | cancer | | | | | | | | |
| AGTA- | D1 | Pancreas | Pancreatic | 40 | 30 | 10 | 20 | 60 | 110 | Y | rare nuclei |
| 90 | | | cancer | | | | | | | | positive |
| AGTA- | D6 | Pancreas | Pancreatic | 40 | 0 | 0 | 60 | 60 | 180 | Y | rare nuclei |
| 90 | | | cancer | | | | | | | | positive |
| AGTA- | D8 | Pancreas | Pancreatic | 75 | 0 | 0 | 25 | 25 | 75 | Y | rare nuclei |
| 90 | | | cancer | | | | | | | | positive |
| AGTA- | D11 | Pancreas | Pancreatic | 30 | 30 | 0 | 40 | 70 | 150 | Y | rare nuclei |
| 90 | | | cancer | | | | | | | | positive |
| AGTA- | D12 | Pancreas | Pancreatic | 98 | 0 | 0 | 2 | 2 | 6 | Y | rare nuclei |
| 90 | | | cancer | | | | | | | | positive |
| AGTA- | D17 | Pancreas | Pancreatic | 30 | 40 | 0 | 30 | 70 | 130 | Y | rare nuclei |
| 90 | | | cancer | | | | | | | | positive |
| AGTA- | D18 | Pancreas | Pancreatic | 20 | 40 | 0 | 30 | 70 | 130 | Y | rare nuclei |
| 90 | | | cancer | | | | | | | | positive |
| AGTA- | D19 | Pancreas | Pancreatic | 10 | 30 | 0 | 60 | 90 | 210 | Y | rare nuclei |
| 90 | | | cancer | | | | | | | | positive |
| AGTA- | E6 | Pancreas | Pancreatic | 75 | 0 | 10 | 15 | 25 | 65 | N | |
| 90 | | | cancer | | | | | | | | |
| AGTA- | E9 | Pancreas | Pancreatic | 100 | 0 | 0 | 0 | 0 | 0 | N | |
| 90 | | | cancer | | | | | | | | |
| AGTA- | E11 | Pancreas | Pancreatic | 25 | 20 | 15 | 10 | 45 | 80 | Y | rare nuclei |
| 90 | | | cancer | | | | | | | | positive |

APPENDIX 3
Globo-H IHC Results in a Normal Tissue TMA - MN1021

| | | | | | | | | Nuclear | |
|---|---|---|---|---|---|---|---|---|---|
| | | | | | | Total | | Staining | |
| Core | | Percentage of Tumor Cells | | | | Percent | H- | Observed? | |
| ID | Organ | 0 | 1+ | 2+ | 3+ | Positive | score | Y/N | Comments |
| A1 | Adrenal gland | 100 | 0 | 0 | 0 | 0 | 0 | N | |
| A2 | Adrenal gland | 100 | 0 | 0 | 0 | 0 | 0 | N | |
| A3 | Adrenal gland | 100 | 0 | 0 | 0 | 0 | 0 | N | |
| A4 | Bladder, urnary | 100 | 0 | 0 | 0 | 0 | 0 | N | endothelial |
| | | | | | | | | | cell postive |
| A5 | Bladder, urnary | 60 | 0 | 40 | 0 | 40 | 80 | N | |
| A6 | Bladder, urnary | 100 | 0 | 0 | 0 | 0 | 0 | N | |
| A7 | Bone, bone | 75 | 0 | 0 | 25 | 25 | 75 | N | |
| | marrow | | | | | | | | |
| A8 | Bone, bone | 100 | 0 | 0 | 0 | 0 | 0 | N | |
| | marrow | | | | | | | | |
| A9 | Head and neck, | 50 | 10 | 10 | 30 | 50 | 120 | N | |
| | salivary gland | | | | | | | | |
| A10 | Head and neck, | 30 | 20 | 10 | 40 | 70 | 160 | N | |
| | salivary gland | | | | | | | | |
| A11 | Eye | 100 | 0 | 0 | 0 | 0 | 0 | N | |
| A12 | Eye | 100 | 0 | 0 | 0 | 0 | 0 | N | |
| A13 | Breast | 90 | 0 | 10 | 0 | 10 | 20 | N | |
| B1 | Breast | 100 | 0 | 0 | 0 | 0 | 0 | N | Scant tissue |
| B2 | Breast | 95 | 0 | 5 | 0 | 5 | 10 | N | |
| B3 | Brain, cerebellum | 100 | 0 | 0 | 0 | 0 | 0 | N | |
| B4 | Brain, cerebellum | 100 | 0 | 0 | 0 | 0 | 0 | N | |
| B5 | Brain, cerebellum | 100 | 0 | 0 | 0 | 0 | 0 | N | |
| B6 | Brain, cerebral | 100 | 0 | 0 | 0 | 0 | 0 | N | |
| | cortex | | | | | | | | |
| B7 | Brain, cerebral | 100 | 0 | 0 | 0 | 0 | 0 | N | |
| | cortex | | | | | | | | |
| B8 | Brain, cerebral | 100 | 0 | 0 | 0 | 0 | 0 | N | |
| | cortex | | | | | | | | |
| B9 | Fallopian tube | 70 | 10 | 10 | 10 | 30 | 60 | N | |
| B10 | Fallopian tube | 80 | 10 | 5 | 5 | 20 | 35 | N | |
| B11 | Fallopian tube | 0 | 20 | 30 | 50 | 100 | 230 | N | |
| B12 | Esophagus | 30 | 0 | 70 | 0 | 70 | 140 | N | |
| B13 | Esophagus | 70 | 0 | 30 | 0 | 30 | 60 | N | |
| C1 | Esophagus | 100 | 0 | 0 | 0 | 0 | 0 | N | |
| C2 | Stomach | 100 | 0 | 0 | 0 | 0 | 0 | N | |
| C3 | Stomach | 100 | 0 | 0 | 0 | 0 | 0 | N | |
| C4 | Stomach | 60 | 10 | 20 | 10 | 40 | 80 | N | |
| C5 | Intestine, small | 100 | 0 | 0 | 0 | 0 | 0 | N | |
| | intestine | | | | | | | | |
| C6 | Intestine, small | 95 | 5 | 0 | 0 | 5 | 5 | N | |
| | intestine | | | | | | | | |
| C7 | Intestine, small | 50 | 10 | 20 | 20 | 50 | 110 | N | |
| | intestine | | | | | | | | |
| C8 | Intestine, colon | 100 | 0 | 0 | 0 | 0 | 0 | N | |
| C9 | Intestine, colon | 70 | 10 | 10 | 10 | 30 | 60 | N | |
| C10 | Intestine, colon | 100 | 0 | 0 | 0 | 0 | 0 | N | |
| C11 | Intestine, rectum | 100 | 0 | 0 | 0 | 0 | 0 | N | |
| C12 | Intestine, rectum | 100 | 0 | 0 | 0 | 0 | 0 | N | |
| C13 | Intestine, rectum | 100 | 0 | 0 | 0 | 0 | 0 | N | |
| D1 | Heart | 100 | 0 | 0 | 0 | 0 | 0 | N | |
| D2 | Heart | 100 | 0 | 0 | 0 | 0 | 0 | N | |
| D3 | Heart | 99 | 1 | 0 | 0 | 1 | 1 | N | |
| D4 | Kidney, cortex | 97 | 1 | 2 | 0 | 3 | 5 | N | |
| D5 | Kidney, cortex | 95 | 5 | 0 | 0 | 5 | 5 | N | |
| D6 | Kidney, cortex | 80 | 10 | 10 | 0 | 20 | 30 | N | |
| D7 | Kidney, medulla | 70 | 10 | 10 | 10 | 30 | 60 | N | |
| D8 | Kidney, medulla | 99 | 1 | 0 | 0 | 1 | 1 | N | |
| D9 | Peripheral nerve | 100 | 0 | 0 | 0 | 0 | 0 | N | |
| D10 | Peripheral nerve | 100 | 0 | 0 | 0 | 0 | 0 | N | |
| D11 | Ureter | 75 | 25 | 0 | 0 | 25 | 25 | n | |
| D12 | Ureter | 100 | 0 | 0 | 0 | 0 | 0 | N | |
| D13 | Liver | N/A | N/A | N/A | N/A | N/A | N/A | N/A | Missing tissue |
| E1 | Liver | 100 | 0 | 0 | 0 | 0 | 0 | N | |
| E2 | Liver | 100 | 0 | 0 | 0 | 0 | 0 | N | |
| E3 | Lung | 100 | 0 | 0 | 0 | 0 | 0 | N | |
| E4 | Lung | 98 | 2 | 0 | 0 | 2 | 2 | N | |
| E5 | Lung | 95 | 0 | 5 | 0 | 5 | 10 | N | |
| E6 | Ovary | 100 | 0 | 0 | 0 | 0 | 0 | N | |
| E7 | Ovary | 100 | 0 | 0 | 0 | 0 | 0 | N | |
| E8 | Ovary | 100 | 0 | 0 | 0 | 0 | 0 | N | |
| E9 | Pancreas | 0 | 0 | 100 | 0 | 100 | 200 | Y | |
| E10 | Pancreas | 30 | 30 | 20 | 20 | 70 | 130 | N | |
| E11 | Pancreas | 0 | 0 | 100 | 0 | 100 | 200 | N | |
| E12 | Parathyroid | 100 | 0 | 0 | 0 | 0 | 0 | N | |
| E13 | Parathyroid | 50 | 0 | 50 | 0 | 50 | 100 | N | |
| F1 | Pituitary gland | 70 | 10 | 10 | 10 | 30 | 60 | Y | Occasional |
| F2 | Pituitary gland | 50 | 30 | 20 | 0 | 50 | 70 | N | |
| F3 | Placenta | 100 | 0 | 0 | 0 | 0 | 0 | N | |
| F4 | Placenta | 100 | 0 | 0 | 0 | 0 | 0 | N | |
| F5 | Placenta | 100 | 0 | 0 | 0 | 0 | 0 | N | |
| F6 | Prostate | 95 | 0 | 5 | 0 | 5 | 10 | N | |
| F7 | Prostate | 99 | 1 | 0 | 0 | 1 | 1 | N | |
| F8 | Prostate | 100 | 0 | 0 | 0 | 0 | 0 | N | |
| F9 | Skin | 100 | 0 | 0 | 0 | 0 | 0 | N | |
| F10 | Skin | 100 | 0 | 0 | 0 | 0 | 0 | N | |
| F11 | Skin | 100 | 0 | 0 | 0 | 0 | 0 | N | |
| F12 | Spinal cord | 100 | 0 | 0 | 0 | 0 | 0 | N | |
| F13 | Spinal cord | 100 | 0 | 0 | 0 | 0 | 0 | N | |
| G1 | Spleen | 100 | 0 | 0 | 0 | 0 | 0 | N | |
| G2 | Spleen | 99 | 1 | 0 | 0 | 1 | 1 | N | |
| G3 | Spleen | 100 | 0 | 0 | 0 | 0 | 0 | N | |
| G4 | Skeletal muscle | 100 | 0 | 0 | 0 | 0 | 0 | N | |
| G5 | Skeletal muscle | 100 | 0 | 0 | 0 | 0 | 0 | N | |
| G6 | Skeletal muscle | 100 | 0 | 0 | 0 | 0 | 0 | N | |
| G7 | Testis | 100 | 0 | 0 | 0 | 0 | 0 | N | |
| G8 | Testis | 100 | 0 | 0 | 0 | 0 | 0 | N | |
| G9 | Testis | 100 | 0 | 0 | 0 | 0 | 0 | N | |
| G10 | Thymus | 99 | 1 | 0 | 0 | 1 | 1 | Y | |
| G11 | Thymus | 97 | 0 | 3 | 0 | 3 | 6 | N | |
| G12 | Thymus | 99 | 0 | 1 | 0 | 1 | 2 | N | |
| G13 | Thyroid | N/A | N/A | N/A | N/A | N/A | N/A | N/A | Missing tissue |
| H1 | Thyroid | 100 | 0 | 0 | 0 | 0 | 0 | N | |
| H2 | Thyroid | 100 | 0 | 0 | 0 | 0 | 0 | N | |
| H3 | Tonsil | 100 | 0 | 0 | 0 | 0 | 0 | N | |
| H4 | Tonsil | 95 | 2 | 3 | 0 | 5 | 8 | N | |
| H5 | Tonsil | 99 | 1 | 0 | 0 | 1 | 1 | N | |
| H6 | Uterus, cervix | 99 | 1 | 0 | 0 | 1 | 1 | N | |
| H7 | Uterus, cervix | 50 | 0 | 50 | 0 | 50 | 100 | N | |
| H8 | Uterus, cervix | 0 | 0 | 0 | 100 | 100 | 300 | N | |
| H9 | Uterus, | 100 | 0 | 0 | 0 | 0 | 0 | N | |
| | endometrium | | | | | | | | |
| H10 | Uterus, | 100 | 0 | 0 | 0 | 0 | 0 | N | |
| | endometrium | | | | | | | | |
| H11 | Uterus, | 95 | 0 | 0 | 5 | 5 | 15 | N | |
| | endometrium | | | | | | | | |

### Appendix 4: Globo-H Ceramide Relative Content in HPAC and SKBR3 Cell Line (Sponsor Data)

The relative content of Globo-H ceramide in HPAC and SKBR3 was estimated by LC-MS/MS. 1×10⁷ cell for each cell line was used and Globo-H ceramide was extracted from cell by MeOH/Chloroform. m/z 1536.0 is selected as the precursor ion and is eluted at 12.2 min. The standard MS/MS spectrum is shown in FIG. 9 and product ions are m/z 512, 844.7, and 1006.8. Globo-H ceramide in cell line was identified by (1) the elution time and (2) MS/MS fragmentation profiling. Relative content of Globo-H ceramide was estimated by the signal intensity of selected product ion m/z 512 and normalized by 10 ng of Globo-H ceramide standard.

FIGS. 23A to 23B show the Extract ion chromatogram of 1536→512 of Globo-H ceramide standard (FIG. 23A) and the MS/MS spectrum of Globo-H ceramide (FIG. 23B). Globo-H ceramide was detected and identified in HPAC and its relative amount is 0.31 referred to 10 ng Globo-H ceramide standard. For SKBR3, EIC of 1536→512 has a small peak at 12.2 min and the relative content is about 0.02 referred to standard. However, the content is much less than that in HPAC. It is only 0.06 in SKBR3 compared to HPAC. Although, the MS/MS fragmentation profiling from SKBR3 is not constituency to the standard spectrum (FIGS. 23A and 23B) which could not be identified by the software, the unique product ions of Globo-H ceramide (m/z 512, 844.7, and 1006.8) were still detected in SKBR3. In conclusion, HPAC is Globo-H ceramide-positive and SKBR3 contains extremely rare Globo-H ceramide which is much less than HPAC.

FIGS. 24A to 24B show the results of LC-MS/MS of HPAC (FIG. 24A) and SKBR3 (FIG. 24B), the upper plot is EIC of 1536→512; the lower plot is the MS/MS spectrum.

The relative content and of Globo-H ceramide in HPAC and SKBR3.

| | | Normalized | Rel. | Quantification | identification |
|---|---|---|---|---|---|
| | intensity | by ref. std. | content | ng per | by MS/MS |
| | [1536.0→512.0] | (10 ng) | to HPAC | 1 × 107 cell | profiling* |
| HPAC | 1.27E+05 | 0.31 | 100% | 12.4 | Y; score 642/1000 |
| SKBR3 | 1.00E+04 | 0.02 | 6% | 0.8 | N |

*The identification score is calculated by the software of DataAnalysis from Bruker. Y means the profiling is consistency to standard. N means that the profiling could not be identified by the software.

### Appendix 5: Globo-H Monoclonal Antibody (VK9) Data Sheet—ThermoFisher Scientific—Cat #14-9700-82

### Appendix 6: EnVision FLEX+, Mouse, High pH (Link) Data Sheet—Agilent (Dako)—Cat #SK8002

### En Vision™ FLEX+, Mouse, High pH, (Link)

### Code K8002

### 5th Edition

The kit contains reagents sufficient for 400-600 tests. For Autostainer Link instruments.

### Optional Reagents:

| Code | Product name | Tests |
|---|---|---|
| K8004 | EnVision ™ FLEX Target Retrieval Solution, High pH (50x) | 200-430 |
| K8005 | EnVision ™ FLEX Target Retrieval Solution, Low pH (50x) | 200-430 |
| K8006 | EnVision ™ FLEX Antibody Diluent | 400-600 |
| K8007 | EnVision ™ FLEX Wash Buffer (20x) | |
| K8008 | EnVision ™ FLEX Hematoxylin, (Link) | 400-600 |
| K8009 | EnVision ™ FLEX+ Rabbit (LINKER), (Link) | 130-200 |
| K8021 | EnVision ™ FLEX+ Mouse (LINKER), (Link) | 130-200 |
| Other options/Autres options/Andere Optionen: | | |
| K8020 | FLEX IHC Microscope Slides | 500 |

### Intended Use

For in vitro diagnostic use.

Dako Envision™ FLEX+ detection system is intended for use m immunohistochemistry together with Autostainer Link instruments. The system detects primary mouse and rabbit antibodies and the reaction is visualized by EnVision™ FLEX DAB+ Chromogen. If used with EnVision™ FLEX+ Mouse (LINKER) or EnVision™ FLEX+ Rabbit (LINKER) (Code K8009) signal amplification of primary mouse antibodies or primary rabbit antibodies, respectively, can be achieved. The EnVision™ FLEX+ reagents are intended for use on formalin-fixed, paraffin-embedded tissue sections.

### Summary and Explanation

Dako EnVision™ FLEX+ detection system, has been designed to be a flexible system and gives an optimal staining on Autostainer Link instruments, when using the protocol recommended in this package insert. As a guideline, using EnVision™ FLEX+ Mouse (Code K8002) signal amplification increases 4-5 fold and using En Vision™ FLEX+ Rabbit (Code K8009, optional reagent) increases signal amplification 2-3 fold.

Prior to staining formalin-fixed, paraffin-embedded tissue sections should be subjected to deparaffinization and hydration followed by heat-induced epitope retrieval (HIER) using the target retrieval method specified in the package insert for the primary antibody. Please also see Procedure section for 3-in-1 specimen preparation. Code K8002 includes En Vision™ FLEX Target Retrieval Solution, High pH (50×). Alternatively, EnVision™ FLEX Target Retrieval Solution, High pH (50×) (Code K8004) or EnVision™ FLEX Target Retrieval Solution, Low pH (50×) (Code K8005) is recommended Some primary antibodies require enzymatic pre-treatment of tissue for optimal staining instead of HIER.

Endogenous peroxidase should be blocked with EnVision™ FLEX Peroxidase-Blocking Reagent (SM801) included in the kit. Due to an effective washing procedure and the presence of stabilizing proteins in the Dako reagents, extra blocking steps to reduce non-specific background staining are unnecessary.

En Vision™ FLEX Wash Buffer (20×) (DM831) included in the kit or available as optional reagent (Code K8007) is recommended.

Primary antibodies are not provided with the kit. We recommend the use of FLEX Ready-to-Use Primary Antibodies or Dako concentrated Primary Antibodies En Vision™ FLEX Antibody Diluent (Code K8006) is recommended for the dilution of Dako concentrated Primary Antibodies.

En Vision™ FLEX+ Mouse (LINKER) (DM824) included in the kit or available as optional reagent (Code K8021) may be replaced by En Vision™ FLEX+ Rabbit (LINKER) (Code K8009) when using rabbit primary antibodies EnVision™ FLEX+ Mouse (LINKER) and En Vision™ FLEX+, Rabbit (LINKER) may be applied for an optional signal amplification of mouse and rabbit primary antibodies respectively. As a guideline: EnVision™ FLEX+ Mouse (LINKER) provides 4-5 fold signal amplification and EnVision™ FLEX+ Rabbit (LINKER) provides 2-3 fold signal amplification.

Dako En Vision™ FLEX/HRP detection reagent (SM802) in the kit consists of a dextran backbone to which a large number of peroxidase (HRP) molecules and secondary antibody molecules have been coupled. A unique chemistry is used for the coupling reaction, which permits the binding of up to 100 HRP molecules and up to 20 antibody molecules per backbone.

The substrate system in the kit consists of two components En Vision™ FLEX DAB+ Chromogen (DM827), a concentrated diaminobenzidine (DAB) solution, and EnVision™ FLEX Substrate Buffer (SM803) containing hydrogen peroxide. Before use EnVision™ FLEX DAB+ Chromogen must be diluted in EnVision™ FLEX Substrate Buffer. The substrate system produces a crisp brown end product at the site of the target antigen.

EnVision™ FLEX Hematoxylin (Code K8008) is recommended for counterstaining. The reagent provides a clear blue, nuclear staining.

The stained tissue sections may be mounted with either aqueous or organic-solvent-based mounting medium.

### Reagents

### A. Materials Provided

| | |
|---|---|
| SM801 | EnVision ™ FLEX Peroxidase-Blocking Reagent |
| | 3 × 40 mL, ready-to-use |
| | Phosphate buffer containing hydrogen peroxide, 15 mmol/L NaN₃ and |
| | detergent. |
| SM802 | EnVision ™ FLEX/HRP |
| | 3 × 40 mL, ready-to-use |
| | Dextran coupled with peroxidase molecules and goat secondary antibody |
| | molecules against rabbit and mouse immunoglobulins. In buffered solution |
| | containing stabilizing protein and preservative. |
| DM827 | EnVision ™ FLEX DAB+ Chromogen |
| | 3 × 3 mL |
| | 3,3′-diaminobenzidine tetrahydrochloride in organic solvent. |
| | The color of this reagent may vary from strong violet to colorless without |
| | having any influence on the performance of the kit. |
| SM803 | EnVision ™ FLEX Substrate Buffer |
| | 12 × 20 mL |
| | Buffered solution containing hydrogen peroxide and preservative. |
| DM828 | EnVision ™ FLEX Target Retrieval Solution, High pH (50x) |
| | 9 × 30 mL, 50x concentrated |
| | Tris/EDTA buffer, pH 9 |
| DM831 | EnVision ™ FLEX Wash Buffer (20x) |
| | 4 × 1 L, 20x concentrated |
| | Tris-buffered saline solution containing Tween 20, pH 7.6 (±0.1). |
| SM804 | EnVision ™ FLEX+, Mouse (LINKER) |
| | 3 × 40 mL, ready-to-use |
| | Buffered solution containing stabilizing protein and antimicrobial agent. |

### B. Materials Required but not Provided

Autostainer Link instrument
Dako PT Link
Dako Proteinase K, Ready-to-Use (if necessary)
Dako FLEX Ready-to-Use Primary Antibodies or suitably diluted concentrated primary rabbit or mouse antibodies from Dako
Dako FLEX Ready-to-Use Rabbit or Mouse Universal Negative Control or suitable negative control reagent for the primary antibody
Dako instrumentation utensils
Microscope slides, e g. FLEX IHC Microscope Slides
General laboratory reagents for deparaffinization of paraffin-embedded tissue sections Mounting medium (aqueous or organic-solvent-based) and coverslips

### C. Optional Reagents

| | |
|---|---|
| K8004 | EnVision ™ FLEX Target Retrieval Solution, High pH (50x) |
| DM828 | 3 × 30 mL, 50x concentrated |
| | Tris/EDTA buffer. pH 9 |
| K8005 | EnVision ™ FLEX Target Retrieval Solution, Low pH (50x) |
| DM829 | 3 × 30 mL, 50x concentrated |
| | Citrate buffer, pH 6.1 |
| K8006 | EnVision ™ FLEX Antibody Diluent |
| DM830 | 120 mL, ready-to-use |
| | Tris buffer, pH 7.2, containing 15 mmol/L NaN₃, and protein. |
| K8007 | EnVision ™ FLEX Wash Buffer (20x) |
| DM831 | 1 L, 20x concentrated |
| | Tris-buffered saline solution containing Tween 20, pH 7.6 (±0.1). |
| K8008 | EnVision ™ FLEX Hematoxylin |
| SM806 | 3 × 45 mL, ready-to-use |
| | Aqueous solution of hematoxylin. |
| K8009 | EnVision ™ FLEX+ Rabbit (LINKER) |
| SM805 | 40 mL, ready-to-use |
| | Buffered solution containing stabilizing protein and an antimicrobial agent. |
| | Contains reagent sufficient for 130-200 tests based on the use of 200-300 μL |
| | per test. |
| K8021 | EnVision ™ FLEX+ Mouse (LINKER) |
| SM804 | 40 mL, ready-to-use |
| | Buffered solution containing stabilizing protein and an antimicrobial agent. |
| | Contains reagent sufficient for 130-200 tests based on the use of 200-300 μL |
| | per test. |

### Precautions

1. For professional users
2. En Vision™ FLEX/HRP, En Vision™ FLEX+ Mouse (LINKER), and En Vision™ FLEX+ Rabbit (LINKER) contain material of animal origin and it cannot be excluded that trace amounts of human material could be present due to manufacturing procedures. As with any product derived from biological sources, proper handling should be used.
3. Do not expose En Vision™ FLEX Peroxidase-Blocking Reagent, En Vision™ FLEX/HRP, En Vision™ FLEX Substrate Buffer, EnVision™ FLEX DAB+ Chromogen, En Vision™ FLEX Substrate Working Solution or EnVision™ FLEX Hematoxylin to strong light during the procedure.
4. EnVision™ FLEX Target Retrieval Solution, High pH (50×) and EnVision™ FLEX Target Retrieval Solution, Low pH (50×) contain 5-<10% Nonoxinol and are labeled:
5. EnVision™ FLEX Wash Buffer (20×) contains 10-30% 2-Amino-2-(hydroxymethyl) propane-1,3-diol hydrochloride and is labeled:
6. For EnVision™ FLEX Peroxidase-Blocking Reagent, a safety data sheet is available for professional users on request.
7. EnVision™ FLEX DAB+ Chromogen contains 1-5% 3,3′-diaminobenzidine tetrahydrochloride, and is labeled:
8. EnVision™ FLEX Antibody Diluent (Code K8006) contains sodium azide (NaN₃), a chemical highly toxic in pure form. At product concentrations, though not classified as hazardous, sodium azide may react with lead and copper plumbing to form highly explosive build-ups of metal azides. Upon disposal, flush with large volumes of water to prevent metal azide build-up in plumbing.
9. Wear appropriate Personal Protective Equipment to avoid contact with eyes and skin.
10. Unused solution should be disposed of according to local, State and Federal regulations.

### Storage

Store EnVision™ FLEX and EnVision™ FLEX+ reagents at 2-8° C.

Store En Vision™ FLEX Peroxidase-Blocking Reagent, En Vision™ FLEX/HRP and En Vision™ FLEX Substrate Buffer in the dark at 2-8° C.

Store En Vision™ FLEX Hematoxylin (Code K8008) in the dark at room temperature. Do not use after expiration date stamped on the vials. If reagents are stored under any conditions other than those specified, the conditions must be verified by the user. The prepared Substrate Working Solution should be stored in the dark at 2-8° C. and used within 5 days. If unexpected staining is observed which cannot be explained by variations in laboratory procedures and a problem with the product is suspected, contact Dako Technical Services.

### Reagent Preparation

### A.1 En Vision™ FLEX Target Retrieval Solution, High pH (50×) (DM828, Code K8004)

Dilute a sufficient quantity of EnVision™ FLEX Target Retrieval Solution, High pH (50×) 1:50 using distilled or deionised water for the staining procedure that is planned. If Dako PT Link is used for pre-treatment, dilution can be performed by emptying the content of the Target Retrieval Solution (50×) vial into the Dako PT Link tank and adding distilled or deionised water to the marked line (Fill Line). Unused diluted solution may be stored at 2-8° C. for one month. Discard solution if cloudy in appearance When used in PT Link for 3-in-1 specimen preparation procedure, the diluted solution can be used 3 times within a 5 day period if stored at room temperature.

### A.2 En Vision™ FLEX Target Retrieval Solution, Low pH (50×) (DM829, Code K8005)

Dilute a sufficient quantity of En Vision™ FLEX Target Retrieval Solution, Low pH (50×) 1:50 using distilled or deionised water for the staining procedure that is planned. If Dako PT Link is used for pre-treatment. dilution can be performed by emptying the content of the Target Retrieval Solution (50×) vial into the Dako PT Link tank and adding distilled or deionised water to the marked line (Fill Line). Unused diluted solution may be stored at 2-8° C. for one month. Discard solution if cloudy in appearance. When used in PT Link for 3-in-1 specimen preparation procedure, the diluted solution can be used 3 times within a 5 day period if stored at room temperature.

### A.3 En Vision™ FLEX Wash Buffer (20×) (DM831, Code K8007)

Dilute a sufficient quantity of EnVision™ FLEX Wash Buffer (20×) 1:20 using distilled or deionised water for the staining procedure that is planned. Dilute by adding the concentrated Wash Buffer to a pre-measured quantity of distilled or deionised water to minimize foaming. Stir gently until the diluted solution appears homogenous. Unused diluted solution may be stored at 2-8° C. for one month. Discard solution if cloudy in appearance. When used in PT Link for 3-in-1 specimen preparation procedure, the diluted solution can be used 3 times within a 5 day period if stored at room temperature.

### A.4 En Vision™ FLEX Substrate Working Solution

The DAB-containing En Vision™ FLEX Substrate Working Solution is prepared by mixing it thoroughly with 1 drop En Vision™ FLEX DAB+ Chromogen (DM827) per 1 mL En Vision™ FLEX Substrate Buffer (SM803). Use En Vision™ FLEX Substrate Working Solution within 5 days (store in the dark at 2-8° C.).

### Specimen Collection and Preparation

The specimens may be formalin-fixed, paraffin-embedded tissue sections. Fixation time is dependent on fixative and tissue type/thickness. For example, tissue blocks with a thickness of 3-4 mm should be fixed in neutral-buffered formalin for 18-24 hours. The optimal thickness of paraffin-embedded sections is approximately 4 μm.

The specimens should be mounted on microscope slides, e g. FLEX IHC Microscope Slides (Code K8020). The sections should be mounted on the slides as flat and wrinkle-free as possible. Too many wrinkles will have an impact on the staining results.

NOTE: The microscope slides must have a width suitable for the Autostainer Link instrument. Please refer to the Operator's Manual for the individual Dako instrument for definition of usable microscope slides.

Paraffin sections should be mounted from a pre-heated water bath containing distilled or deionized water. The water bath should contain no additives (such as gelatin, poly-L-lysine etc.). Sections should be dried by heating, generally at a temperature not above 60° C. for up to 60 minutes. To ensure proper adherence of sections to slides it is important to drain the water from beneath the sections prior to the oven drying process.

### Procedure

Autostainer Link instruments use techniques based on different principles to obtain an optimal staining result. Before running protocols on your Autostainer Link instrument, please read carefully the Operator's Manual for the dedicated Dako Instrument.

In order to ascertain reproducible epitope retrieval it is recommended always to load the slide holder fully with slides. This ensures an identical heating of sections in every run.

### A. Pre-Treatment Procedure

### Recommended 3-In-1 Specimen Preparation Procedure Using PT Link:

Deparaffinization, rehydration and heat-induced epitope retrieval (HIER) can be performed on formalin-fixed paraffin-embedded tissue sections using the 3-in-1 procedure:
1. Prepare a working solution by diluting the Envision™ FLEX Target Retrieval Solution (50×) concentrate 1:50 in distilled or deionized water.
2. Fill PT Link tanks with sufficient quantity (1.5 L) of working solution to cover the tissue sections.
3. Set PT Link to pre-heat the solution to 65° C.
4. Immerse the mounted, formalin-fixed, paraffin-embedded tissue sections into the pre-heated EnVision™ FLEX Target Retrieval Solution (working solution) in PT Link tanks and incubate for 20-40 minutes at 97° C. The optimal incubation time should be determined by the user.
5. Leave the sections to cool in PT Link to 65° C.
6. Remove each Autostainer slide rack with the slides from the PT Link tank and immediately dip slides into a jar/tank (e.g., PT Link Rinse Station, Code, PT109) with diluted, room temperature EnVision™ FLEX Wash Buffer (20×).
7. Leave slides in the diluted, room temperature En Vision™ FLEX Wash Buffer (20×) for 1-5 minutes.
8. Place slides on an Autostainer Link instrument and proceed with staining. The sections should not dry out during the treatment or during the immunohistochemical staining procedure.
9. After staining it is recommended to perform dehydration, clearing and permanent mounting.

NOTE: When used in PT Link for 3-in-1 specimen preparation procedure, the diluted Target Retrieval Solution and diluted Wash Buffer can be used 3 times within a 5 day period if stored at room temperature.

### HIER Procedure Using PT Link:

After deparaffinization and hydration to buffer (water), the tissue sections should be subjected to heat-induced epitope retrieval (HIER):
1. Prepare a working solution by diluting the EnVision™ FLEX Target Retrieval Solution (50×) concentrate 1:50 in distilled or deionized water.
2. Fill PT Link tanks with sufficient quantity (1.5 L) of working solution to cover the tissue sections.
3. Set PT Link to pre-heat the solution to 65° C.
4. Manually deparaffinize and rehydrate tissue sections mounted on glass slides. For better adhesion of tissue to slide, use of coated or silanized slides is recommended.
5. Immerse room temperature tissue sections into the pre-heated EnVision™ FLEX Target Retrieval Solution (working solution), and incubate for 20-40 minutes at 97° C. The optimal incubation time should be determined by the user.
6. Leave the sections to cool in PT Link to 65° C.
7. Remove each slide rack from PT Link tank and immediately dip slides into a jar/tank (e.g., PT Link Rinse Station, Code PT109) with diluted, room temperature EnVision™ FLEX Wash Buffer (20×).
8. Leave slides in the diluted, room temperature EnVision™ FLEX Wash Buffer (20×) for 1-5 minutes.
9. Place slides on an Autostainer Link instrument and proceed with staining. The sections should not dry out during the treatment or during the immunohistochemical staining procedure.
10. After staining the slides should be mounted using aqueous or permanent mounting medium.

NOTE: When used in PT Link for HIER procedure, the diluted Target Retrieval Solution and diluted Wash Buffer can be used 3 times within a 5 day period if stored at room temperature.

### HIER Procedure Using Coplin Jars:

After deparaffinization and hydration to buffer (water), the tissue sections should be subjected to heat-induced epitope retrieval (HIER):
1. Place staining jars containing diluted EnVision™ FLEX Target Retrieval Solution (50×) in water bath.
2. Heat water bath and jars filled with EnVision™ FLEX Target Retrieval Solution (working solution) to 95-99° C. Cover jars with lids to stabilize the temperature and avoid evaporation.
3. Immerse the room temperature sections in the preheated EnVision™ FLEX Target Retrieval Solution (working solution) in the staining jars.
4. Bring temperature of the water bath and EnVision™ FLEX Target Retrieval Solution back to 95-99° C. Incubate for 20 (+1) minutes at 95-99° C.
5. Remove the entire jar with slides from the water bath. Allow slides to cool in the EnVision™ FLEX Target Retrieval Solution for 20 (+1) minutes at room temperature.
6. Decant the En Vision™ FLEX Target Retrieval Solution and rinse sections with diluted, room temperature EnVision™ FLEX Wash Buffer (20×) for 1-5 minutes.
7. Place the rack with rinsed sections on an Autostainer Link instrument. Immediately begin the preprogrammed staining run.

NOTE: When used in Coplin jars HIER procedure, the diluted Target Retrieval Solution and diluted Wash Buffer can be used only 1 time (single use).

A few epitopes do not tolerate the HIER and some require enzymatic pre-treatment. Please refer to the package insert for the individual Dako primary antibody.

The tissue sections should not dry out during the treatment or during the following immunohisto-chemical staining procedure. For greater adherence of tissue sections to glass slides, the use of FLEX IHC Microscope Slides (Code K8020) is recommended.

### B. Staining Procedure

Dako FLEX Ready-to-Use Primary Antibodies can be used with Dako En Vision™ FLEX+ detection system, applied on formalin-fixed, paraffin-embedded tissue sections.

A dilution guideline for Dako concentrated primary antibodies is provided in the package insert of the concentrated primary antibody.

The concentrated primary antibodies should be diluted in En Vision™ FLEX Antibody Diluent (DM830, Code K8006)

The staining steps and recommended incubation times are pre-programmed into the Autostainer Link software as the following visualization system protocols:
FLEX and FLEX 2×5 DAB (FLEX protocols)
FLEX+ Mouse and FLEX+ Mouse 2×5 DAB (FLEX+ Mouse (LINKER) protocols)
FLEX+ Rabbit and FLEX+ Rabbit 2×5 DAB (FLEX+ Rabbit (LINKER) protocols)

The recommended reagent application volume is 1×200 μL or 2×150 μL per slide. If the protocols are not available on the used Autostainer Link instrument, please contact Dako Technical Services.

Optimal incubation times of primary antibody and EnVision™ FLEX/HRP are dependent on the applied primary antibody. Please refer to the package insert for the individual Dako primary antibody The user must verify the applied protocol.

When the staining procedure is completed, the specimens must be mounted. It is recommended to perform dehydration, clearing and permanent mounting when slides have been treated with the 3-in-1 specimen preparation procedure. Slides treated with HIER procedure can be mounted using aqueous or permanent mounting medium. For aqueous mounting, mounting media such as Dako Glycergel™ Mounting Medium, Code C0563, or Faramount Aqueous Mounting Medium Code S3025 is recommended.

### Quality Control

Each staining run should include a known positive control specimen to ascertain a proper performance of all the applied reagents. If the positive control specimen fails to demonstrate positive staining, labeling of test specimens should be considered invalid.

A negative control reagent should be used with each specimen to identify any non-specific staining If non-specific staining cannot be dearly differentiated from the specific staining, the labeling of the test specimen should be considered invalid

### Interpretation of Results

The diaminobenzidine-containing Substrate Working Solution gives a brown color at the site of the target antigen recognized by the primary antibody The brown color should be present on the positive control specimen at the expected localization of the target antigen If non-specific staining is present, this will be recognized as a rather diffuse, brown staining on the slides treated with the negative control reagent Nuclei will be stained blue by the hematoxylin counterstain.

### Appendix 7: EnVision FLEX, Low pH (Link) Data Sheet—Agilent (Dako)—Cat #SK8005

### En Vision™ FLEX (Link)

### Optional Reagents for Autostainer Link Instruments

### Code K8004, K8005, K8006, K8007, K8008, K8009, K8021

These reagents are described in the package inserts for:
Code K8000: EnVision™ FLEX, High pH (Link)
Code K8002: EnVision™ FLEX+, High pH (Link)
Code K8023: En Vision™ FLEX Mini kit, High pH (Link)

### En Vision™ FLEX (Dako Autostainer/Autostainer Plus)

### Optional Reagents for Dako Autostainer Instruments

### Code K8004, K8005, K8006, K8007, K8018, K8019, K8022

These reagents are described in the package inserts for:
Code K8010: EnVision™ FLEX, High pH (Dako Autostainer/Autostainer Plus)
Code K8012: EnVision™ FLEX+, High pH (Dako Autostainer/Autostainer Plus)
Code K8024: EnVision™ FLEX Mini kit, High pH (Dako Autostainer/Autostainer Plus)

What is claimed is:

1. A method of identifying a patient eligible for Globo-H mediated therapy, the method comprising:
(a) providing or having provided a tissue sample of breast cancer from the patient;
(b) contacting the tissue sample with a Globo-H antibody, wherein the Globo-H antibody is a VK9 antibody;
(c) forming a plurality of complexes, each comprising a Globo-H antigen-antibody, wherein the plurality of complexes comprises a first complex located at a cell membrane and a second complex located in a cell cytoplasm of the tissue sample;
(d) contacting the complex with a detectably labeled second antibody that binds to the Globo-H antibody thereby forming labeled complexes;
(e) generating a detectable signal associated with both the first complex and the second complex;
(f) detecting the detectable signal using immunohistochemistry (IHC) and relating the units of detectable signal to the level of Globo-H antigens in the tissue sample; and
(g) selecting patients with a level of Globo-H higher than a threshold.

2. (canceled)

3. The method of claim 1, wherein the tissue sample comprises a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, or a paraffin embedded tissue sample.

4-29. (canceled)


SEQUENCE LISTING (16 sequences)

SEQ ID NO: 1 - protein, 116 aa, Artificial Sequence
  Description of Artificial Sequence Synthetic polypeptide
SGPGILQPSQTLSLTCSFSGFSLYTFDMGVGWIRQPSGKGLEWLAHIWWDDDKYYNPALKSRLTVSKDTSKNQVFLKIPNVDTADSATYYCARVRGLHDYYYWFAYWGQGTLVTVS

SEQ ID NO: 2 - protein, 116 aa, Artificial Sequence; identical to SEQ ID NO: 1
  Description of Artificial Sequence Synthetic polypeptide

SEQ ID NO: 3 - protein, 116 aa, Artificial Sequence
  Description of Artificial Sequence Synthetic polypeptide
SGPGILQPSQTLSLTCSFSGFSLYTFDMGVGWIRQPSGKGLEWLAQIWWDDDKYYNPGLKSRLTISKDTSKNQVFLKIPNVDTADSATYYCARIRGLRDYYYWFAYWGQGTLVTVS

SEQ ID NO: 4 - protein, 117 aa, Artificial Sequence
  Description of Artificial Sequence Synthetic polypeptide
SGPGILQPSQTLSLTCSFSGFSLSTFGLGVGWIRQPSGKGLEWLAHIWWDDDKSYNPALKSRLTISKDTSKNQVFLMIANVDTADTATYYCARIGPKWSNYYYYCDYWGQGTTLTVS

SEQ ID NO: 5 - protein, 117 aa, Artificial Sequence
  Description of Artificial Sequence Synthetic polypeptide
SGPGILQPSQTLSLTCSFSGFSLSTFGLGVGWIRQPSGKGLEWLAHIWWDDDKSYNPALKSQLTISKDTSKNQVLLKIANVDTADTATYYCARIGPKWSNYYYYCDYWGQGTTLTVS

SEQ ID NO: 6 - protein, 112 aa, Artificial Sequence
  Description of Artificial Sequence Synthetic polypeptide
QITLKESGPTLVKPTQTLTLTCTFSGFSLYTFDMGVGWIRQPPGKGLEWLAHIWWDDDKYYNPALKSRLTISKDTSKNQVVLTMTNMDPVDTATYYCARVRGLHDYYYWFAY

SEQ ID NO: 7 - protein, 112 aa, Artificial Sequence
  Description of Artificial Sequence Synthetic polypeptide
QVTLKESGPGILQPSQTLSLTCSFSGFSLYTFDMGVGWIRQPSGKGLEWLAHIWWDDDKYYNPALKSRLTVSKDTSKNQVFLKIPNVDTADSATYYCARVRGLHDYYYWFAY

SEQ ID NO: 8 - protein, 453 aa, Artificial Sequence
  Description of Artificial Sequence Synthetic polypeptide
QITLKESGPTLVKPTQTLTLTCTFSGFSLYTFDMGVGWIRQPPGKGLEWLAHIWWDGDKYYNPALKSRLTISKDTSKNQVVLTMTNMDPVDTATYYCARVRGLHRYYYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 94 aa, Artificial Sequence
  Description of Artificial Sequence Synthetic polypeptide
ASPGEKVTMTCRASSSVSYMHWYQQKPGSSPKPWIYATSNLASGVPARFSGSGSGTSYSLTISRVEAEDAATYFCQQWSRNPFTFGSGTKLEIR

SEQ ID NO: 10 - protein, 94 aa, Artificial Sequence; identical to SEQ ID NO: 9
  Description of Artificial Sequence Synthetic polypeptide

SEQ ID NO: 11 - protein, 94 aa, Artificial Sequence; identical to SEQ ID NO: 9
  Description of Artificial Sequence Synthetic polypeptide

SEQ ID NO: 12 - protein, 94 aa, Artificial Sequence
  Description of Artificial Sequence Synthetic polypeptide
ASPGEKVTMTCRASSSVSYMHWYQQKPGSSPKPYIYATSNLSSGVPARFSGSGSGTSYSLTISRVEAEDAATYYCQQWSSNPFTFGSGTKLEIK

SEQ ID NO: 13 - protein, 94 aa, Artificial Sequence; identical to SEQ ID NO: 12
  Description of Artificial Sequence Synthetic polypeptide

SEQ ID NO: 14 - protein, 96 aa, Artificial Sequence
  Description of Artificial Sequence Synthetic polypeptide
EIVLTQSPATLSLSPGERATLSCRASSSVSYMHWYQQKPGKSPKPWIYATSNLASGVPSRFSGSGSGTDFTFTISSLQPEDIATYYCQQWSRNPFT

SEQ ID NO: 15 - protein, 96 aa, Artificial Sequence
  Description of Artificial Sequence Synthetic polypeptide
QIVLSQSPTILSASPGEKVTMTCRASSSVSYMHWYQQKPGSSPKPWIYATSNLASGVPARFSGSGSGTSYSLTISRVEAEDAATYFCQQWSRNPFT

SEQ ID NO: 16 - protein, 213 aa, Artificial Sequence
  Description of Artificial Sequence Synthetic polypeptide
EIVLTQSPATLSLSPGERATLSCRASSSVSYMHWYQQKPGKSPKPWIYATSNKASGVPSRFSGSGSGTDFTFTISSLQPEDIATYYCQQWSRRPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC